(19) United States
(12) Patent Application Publication
(10) Pub. No.: US 2025/0270248 A1
(43) Pub. Date: Aug. 28, 2025

(54) OLIGONUCLEOTIDES CONJUGATED TO FATTY ACIDS

(71) Applicant: AstraZeneca AB, Södertälje (SE)

(72) Inventors: Shalini ANDERSSON, Södertälje (SE); Daniel Laurent KNERR, Södertälje (SE); Erik MÜLLERS, Södertälje (SE)

(73) Assignee: ASTRAZENECA AB, Södertälje (SE)

(21) Appl. No.: 18/245,422

(22) PCT Filed: Sep. 15, 2021
(86) PCT No.: PCT/EP2021/075386

§ 371 Date: Mar. 15, 2023

Related U.S. Application Data
(60) Provisional application No. 63/079,179, filed on Sep. 16, 2020.

(51) Int. Cl. C07H 21/04; C07H 21/02

(52) U.S. Cl. CPC C07H 21/04; C07H 21/02

(57) ABSTRACT

Provided are lipid conjugated oligonucleotides that allow for delivery of the oligonucleotides to specific tissues in the body. The lipid on the conjugated oligonucleotide comprises an acyl group having a free terminal carboxylic acid group. Also provided are methods for synthesizing lipid conjugated oligonucleotides having with a lipid comprising an acyl group having a free terminal carboxylic acid group, along with methods for delivering the lipid conjugated oligonucleotides and methods for using the lipid conjugated oligonucleotides for treating disease.

### SUMMARY

The present disclosure relates to oligonucleotides conjugated to acyl chains having a terminal carboxyl group. In some embodiments, the disclosure provides methods of making oligonucleotides conjugated to acyl chains having a terminal carboxyl group.

Oligonucleotide based therapies—such as antisense therapies, gene therapies and CRISPR gene editing therapies—are thought to hold promise for treatment of various conditions. However, delivery of oligonucleotides to specific tissues in the body has been a challenge for oligonucleotide-based therapies.

One strategy for delivering oligonucleotides to specific tissues is to deliver the oligonucleotides packaged into lipid nanoparticles or polymer nanoparticles. Because oligonucleotides, unless specially modified, have a polyanionic backbone, cationic lipids or polymers are used in forming nanoparticles. The electrostatic interaction between the anionic oligonucleotides and cationic lipids or polymers causes the nanoparticles to form.

While a wide variety of lipid nanoparticles has been developed, almost all of these nanoparticles have limitations for use in therapeutics. One limitation is the amount of carrier, i.e., lipid, material that must be delivered to support the administration of oligonucleotides. For example, for small interfering RNAs (siRNAs) packaged in lipid nanoparticles, the siRNA makes up only a few percent of the total mass of the deliverable, with the remaining mass being lipids. Juliano, Nucleic Acids Research 2016, 44(14):6518-6548. The delivery of large amount of cationic lipids can be problematic as many biological macromolecules are anionic and thus electrostatically associate with the cationic lipids leading to toxicity issues.

Further, lipid nanoparticles typically only accumulate in tissues having a fenestrated (e.g., porous) endothelium: liver, spleen and some tumor tissues. Osborn et al., Nucleic Acid Therapeutics 2018, 28(3):128-136. This makes the use of lipid nanoparticles impractical for therapies where the oligonucleotide needs to be delivered to other tissues.

Lipid conjugated oligonucleotides are being developed to improve the delivery of oligonucleotides. As the oligonucleotide is chemically linked to the lipid, an electrostatic interaction between the two is not needed and potentially toxic cationic lipids are not required. Further, as a single oligonucleotide molecule is usually conjugated to only one or two lipid chains, the amount of lipid carrier material is substantially reduced. Thus, side effects with lipid conjugated oligonucleotides should be reduced.

As the lipids used for conjugation do not have to be suitable for lipid nanoparticle formation, a larger variety of lipids can be used. By varying the lipid portion of the lipid conjugated oligonucleotide, it is possible to target a wider range of tissues for uptake of the conjugates.

The present disclosure is directed to acid acyl conjugated oligonucleotides comprising:
a) a carboxy acyl group;
b) an oligonucleotide; and
c) a linker connecting the carboxy acyl group to the oligonucleotide.

In some embodiments, the carboxy acyl group is C₄ to C₃₂ and may be saturated, for example, monounsaturated or unsaturated, for instance polyunsaturated.

In some embodiments, the acid acyl conjugated oligonucleotides comprise a linker bound to the 5′ end of the oligonucleotide. In some embodiments, the acid acyl conjugated oligonucleotides comprise a linker comprising an amino terminus connected to the carboxy acyl group. In some embodiments, the acid acyl conjugated oligonucleotides comprise a linker comprising a phosphate terminus connected to the oligonucleotide.

In some embodiments, the acid acyl conjugated oligonucleotides comprise a DNA oligonucleotide. In some embodiments, the acid acyl conjugated oligonucleotides comprise a RNA oligonucleotide. In some embodiments, the acid acyl conjugated oligonucleotides comprise an antisense oligonucleotide. In some embodiments, the oligonucleotide is a phosphorothioate oligonucleotide.

According to the present disclosure, the acid acyl conjugated oligonucleotides of the present disclosure may be chosen from compounds of formula (I):

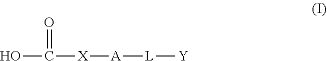

wherein:
X is C₄ to C₃₂ alkyl or alkenyl;
A is a conjugation group;
L is a linker; and
Y is an oligonucleotide.

Also disclosed herein are acid acyl conjugated oligonucleotides of formula (Ia):

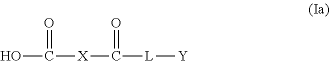

wherein:
X is C₁₀ to C₂₆ alkyl;
L is a linker—NH—C₆H₁₂—O—PO₂—; and
Y is DNA,
   wherein the linker is attached to the DNA via a phosphate linkage at the 5′ end of the DNA.

The present disclosure further comprises methods to improve the reduction in cardiac cell expression compared comprising administering an acid acyl conjugated oligonucleotide disclosed herein to a mammal, wherein the acid acyl conjugated oligonucleotide improves reduction in cardiac cell expression compared to a non-acyl conjugated oligonucleotide.

Also disclosed are methods of reducing expression of a gene of interest in the cardiac cells of a mammal, comprising administering an acid acyl conjugated oligonucleotide disclosed herein to the mammal, wherein the acid acyl conjugated oligonucleotide reduces expression of a gene of interest in cardiac cells compared to a non-acyl conjugated oligonucleotide

Further disclosed herein are methods of making an acid acyl oligonucleotide of formula (Ia), the method comprising:
A) providing a fatty diacid of formula (II)

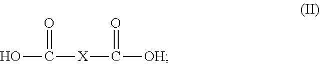

B) reacting the fatty diacid with an activating group, A, to form an acid acyl compound of formula (III):

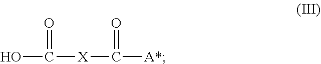

wherein A* is the activating group attached to the fatty acyl compound;
C) reacting the compound of formula (III) with a compound of formula (IV):

*L-Y (IV)

wherein *L is a linker with a reactive group;
to form the compound of formula (Ia):

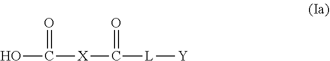

The present disclosure further provides methods of delivering an oligonucleotide to cardiac tissue in a subject comprising:
a) providing acid acyl conjugated oligonucleotide according to the present disclosure, and
b) administering the acid acyl conjugated oligonucleotide to the subject.

In the following description, certain details are set forth in order to provide a thorough understanding of various embodiments. However, one skilled in the art will understand that the disclosed embodiments may be practiced without these details. These and other embodiments will become apparent upon reference to the following detailed description and attached drawings.

### BRIEF DESCRIPTION OF DRAWINGS

FIG. 1 shows the dissociation constants of 5′ lipid conjugated Malat-1 ASOs from Human (HSA) and rat (RSA) serum albumin measured by surface plasmon resonance (SPR) as exemplified in Example 25.

FIG. 2 shows the concentration dependent knock down of Malat-1 gene expression in human THP-1 monocytes after treatment with FA-ASO conjugates as exemplified in Example 26.

FIGS. 3A-D shows the results of Example 27, that the lipidated CamK2D ASOs of Examples 12 (FIG. 3A), Example 13 (FIG. 3B), Example 14 (FIG. 3C), and Example 15 (FIG. 3D) maintained functional activity in vitro.

FIGS. 4A-B shows that conjugation to a saturated C₂₂ acid or C₁₈ (9Z) monounsaturated fatty diacid led to similar or increased knock down in the heart but also to an attenuation of the knock down measured in the liver and kidney compared to the parent ASO with FIG. 4A exemplifying Examples 12 and 15 MALAT-1 gene expression in the heart and FIG. 4B exemplifying Examples 7 and 10 MALAT-1 gene expression in the liver and kidney. ns: Analysis was carried out via two-way ANOVA followed by Bonferroni multiple comparisons. ***: p<0.001, one way ANOVA followed by Dunnett multiple comparisons.

FIG. 5 shows CamK2D gene expression level in the heart, kidney, and liver and shows that the saturated C₂₂ acid chain conjugation improved knock down in the heart and tend to attenuate the knock-down in sink organs like kidney and liver in comparison to the naked parent ASOs as exemplified in Example 29. ** p<0.01. *** p<0.001, two way ANOVA followed by Bonferroni multiple comparisons.

### DETAILED DESCRIPTION

The present disclosure provides lipid conjugated oligonucleotides where the lipid comprises an acyl group and a free terminal carboxylic acid group. The present disclosure includes methods of making lipid conjugated oligonucleotides where the lipid comprises and acyl group and a free terminal carboxylic acid group. The present disclosure includes methods of delivering the lipid conjugate oligonucleotides disclosed herein to a subject. The present disclosure also includes methods of administering the lipid conjugate oligonucleotides disclosed herein to treat disease in a subject.

It should be appreciated that the particular implementations shown and described herein are examples and are not intended to otherwise limit the scope of the application in any way.

As used interchangeably herein, a “nucleic acid,” “nucleic acid molecule,” “nucleotide,” “nucleotide sequence,” “oligonucleotide,” or “polynucleotide” means a polymeric compound including covalently linked nucleotides. A nucleotide includes a nucleoside linked to a phosphate group. A nucleoside includes a nucleobase and sugar moiety. The nucleobase may be naturally occurring or synthetic. The nucleobase and sugar base may each, independently, be modified or unmodified. “Modified nucleoside” means a nucleoside comprising a modified nucleobase and/or a modified sugar moiety. Modified nucleosides can include abasic nucleosides, which lack a nucleobase. Polynucleotides or oligonucleotides may be modified or unmodified and may contain one or more modified nucleosides. “Modified polynucleotide” or “modified oligonucleotide” means a polynucleotide or oligonucleotide, wherein at least one sugar, nucleobase, or internucleoside linkage is modified. In some embodiments, the modified polynucleotide or modified oligonucleotide is oligonucleotide is a phosphorothioate polynucleotide. “Unmodified polynucleotide” means a polynucleotide that does not comprise any sugar, nucleobase, or internucleoside modification. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments the polynucleotide or oligonucleotide is double stranded DNA. In some embodiments the polynucleotide or oligonucleotide is single stranded DNA. In some embodiments the polynucleotide or oligonucleotide is double stranded RNA. In some embodiments the polynucleotide or oligonucleotide is single stranded RNA. In some embodiments the polynucleotide or oligonucleotide is an antisense RNA. Nucleic acids of the present disclosure may be any length. In some embodiments, a nucleic acid provided herein is 8 to 80 nucleotides in length. In some embodiments, a nucleic acid provided herein is 10 to 70 nucleotides in length, 12 to 60 nucleotides in length, 15 to 50 nucleotides in length, 15 to 45 nucleotides in length, 16 to 40 nucleotides in length, 17 to 35 nucleotides in length, 18 to 30 nucleotides in length, 19 to 29 nucleotides in length or 20 to 28 nucleotides in length.

As used herein, the term “antisense molecule” means an oligomeric molecule that is capable of undergoing hybridization to a target nucleic acid, e.g., through hydrogen bonding. Examples of antisense molecules include single-stranded and double-stranded nucleic acids, such as, e.g., antisense oligonucleotides (ASO), small interfering RNAs (siRNA), short hairpin RNAs (shRNA), small nucleolar RNAs (snoRNA), microRNAs (miRNA), and meroduplexes (mdRNA), and satellite repeat sequences.

An “antisense oligonucleotide” or “ASO” refers to a polynucleotide comprising a sequence that is complementary to a target nucleic acid or region or segment thereof. In some embodiments, an ASO is specifically hybridizable to a target nucleic acid or region or segment thereof. In some embodiments, ASOs are capable of influencing RNA processing and/or modulating protein expression. In general, an ASO is a single-stranded oligonucleotide that binds to single-stranded RNA to inactivate the RNA. In some embodiments, an ASO binds to messenger RNA (mRNA) for a gene, thereby inactivating the gene. In some embodiments, an ASO binds to a non-coding mRNA, thereby disrupting the function of the non-coding mRNA. In some embodiments, an ASO binds to a transcription initiation site, a translation initiation site, 5′-untranslated sequence, 3′-untranslated sequence, coding sequence, a pre-mRNA sequence, an mRNA splice site, and/or an intron/exon junction of an mRNA encoding a gene, thereby inactivating the gene. In some embodiments, the ASO includes DNA, RNA, or combination thereof. ASOs are further described in, e.g., Goodchild, Methods Mol Biol 764:1-15, 2011; Smith et al., Ann Rev Pharmacol Toxicol 59:605-630, 2019; and Stein et al., Mol Ther 25(5):1069-1075, 2017.

In embodiments of any of the above, the oligonucleotide can be unmodified DNA, RNA or may be modified. Modified oligonucleotides comprise at least one modification relative to unmodified RNA or DNA (i.e., comprise at least one modified nucleoside (comprising a modified sugar moiety and/or a modified nucleobase and/or at least one modified internucleoside linkage). In embodiments, the oligonucleotide can be selected from any of the oligonucleotides described herein. In embodiments, the oligonucleotide is an antisense oligonucleotide. In embodiments, the antisense oligonucleotide contains at least one phosphorothioate internucleoside linkage. In embodiments, the antisense oligonucleotide contains at least one modified sugar moiety, for example, a bicyclic sugar moiety (e.g., comprising two rings, wherein the second ring is formed via a bridge connecting two of the atoms in the first ring thereby forming a bicyclic structure), such as a furanosyl moiety. In embodiments, the antisense oligonucleotide contains at least one modified nucleobase.

### Carboxy Acyl Groups and Linkers

In embodiments, the present disclosure provides an acyl acid conjugated oligonucleotide comprising a carboxy acyl group connected to an oligonucleotide. In embodiments, the carboxy acyl group is connected to the oligonucleotide by a linker.

In embodiments, the carboxy acyl group of the conjugate is a fatty acid chain having a free terminal carboxylic acid group. In embodiments, the acyl group has a length of from C₄ to C₃₂. In embodiments, the acyl group has a length of from C₆ to C₃₀. In embodiments, the acyl group has a length of from C₈ to C₂₈. In embodiments, the acyl group has a length of from C₁₀ to C₂₆. In embodiments, the acyl group has a length of from C₁₂ to C₂₆. In embodiments, the acyl group has a length of from C₁₄ to C₂₄. In embodiments, the acyl group has a length of from C₁₆ to C₂₂. In embodiments, the acyl group has a length of C₁₆. In embodiments, the acyl group has a length of C₁₈. In embodiments, the acyl group has a length of C₂₂.

In embodiments, the carboxy terminal on the acyl group provides improved uptake of the conjugated oligonucleotide in specific tissues and/or organs upon administration. In embodiments, the carboxy terminal on the acyl group provides improved uptake in cardiac tissue or liver tissue. In embodiments, the carboxyl terminal on the acyl group provides improved uptake in the heart or liver. In embodiments, the carboxyl terminal on the acyl group provides improved uptake in cardiac tissue. In embodiments, the carboxyl terminal on the acyl group provides improved uptake in the heart.

In embodiments, the acyl group is saturated, i.e., having only single carbon-carbon bonds. In embodiments, the acyl group is unsaturated, i.e., having one or more double carbon-carbon bonds. In embodiments, the acyl group is monounsaturated. In embodiments, the acyl group is polyunsaturated, e.g., having 2-15 double bond, e.g., having 2, 3, 4, 5, 6, 7, 8, 9, 10, 11, 12, 13, 14 or 15 double bonds.

In embodiments, the lipid conjugated oligonucleotides can include a single carboxy acyl group attached to the oligonucleotide. In embodiments, the lipid conjugated oligonucleotides can include a two carboxy acyl groups attached to the oligonucleotide. In embodiments, the lipid conjugated oligonucleotides can include a three or more carboxy acyl groups attached to the oligonucleotide. In embodiments, the lipid conjugated oligonucleotides can include a single carboxy acyl group attached to the oligonucleotide along with one, two or more other acyl groups that do not have a terminal carboxy. In embodiments, the lipid conjugated oligonucleotides can include two carboxy acyl groups attached to the oligonucleotide along with one, two or more other acyl groups that do not have a terminal carboxy.

In embodiments, the carboxyl acyl group is attached to the 5′ end of the oligonucleotide. In embodiments, the carboxyl acyl group is attached to the 3′ end of the oligonucleotide. In embodiments, the carboxy acyl group is attached to the oligonucleotide at a modified base in the oligonucleotide instead of the 5′ or 3′ ends of the oligonucleotide.

In embodiments, the carboxyl acyl group is connected to the oligonucleotide by a linker. In embodiments, the linker is bound to the 5′ end of the oligonucleotide. In embodiments, the linker forms an amide bond with the carboxy acyl group. In embodiments, the linker comprises a phosphate linkage connected to the oligonucleotide. In embodiments, the linker is attached to the 3′ end of the oligonucleotide. In embodiments, the linker is attached to the oligonucleotide at a modified base in the oligonucleotide.

The linker may be any chemical moiety that allows for chemical attachment of the carboxy acyl group at one end of the linker and the oligonucleotide at another end of the linker. In embodiments, the linker forms an amide bond with the carboxy acyl group. In embodiments, the linker comprises a phosphate linkage connected to the oligonucleotide. In embodiments, the phosphate linkage of the linker can be a 5′ phosphate on the oligonucleotide.

In embodiments, the linker comprises an acyl chain between termini. In embodiments, the linker comprises an acyl chain of C₁ to C₁₀ in length. In embodiments, the acyl chain can be saturated. In embodiments, the acyl chain can be monounsaturated or polyunsaturated. In embodiments, the linker forms an amide bond with the carboxy acyl group, a phosphate linkage with the oligonucleotide, and an acyl chain of C₁ to C₁₀ in length connecting the amide bond with the phosphate terminal.

In embodiments, the present disclosure provides an acid acyl conjugated oligonucleotide of formula (I)

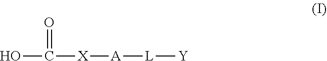

wherein: X is C₄ to C₃₂ alkyl or alkenyl; A is a conjugation group; L is a linker; and Y is an oligonucleotide.

In embodiments of formula (I), X is C₆ to C₃₀ alkyl or alkenyl. In embodiments, X is C₈ to C₂₈ alkyl or alkenyl. In embodiments, X is C₁₀ to C₂₆ alkyl or alkenyl. In embodiments, X is C₁₂ to C₂₆ alkyl or alkenyl. In embodiments, X is C₁₄ to C₂₄ alkyl or alkenyl. In embodiments, X is C₁₄ alkyl or alkenyl. In embodiments, X is C₁₆ alkyl or alkenyl. In embodiments, X is C₂₀ alkyl or alkenyl.

In embodiments of formula (I), X is C₆ to C₃₀ alkyl. In embodiments, X is C₈ to C₂₈ alkyl. In embodiments, X is C₁₀ to C₂₆ alkyl. In embodiments, X is C₁₂ to C₂₆ alkyl. In embodiments, X is C₁₄ to C₂₄ alkyl. In embodiments, X is C₁₄ alkyl. In embodiments, X is C₁₆ alkyl. In embodiments, X is C₂₀ alkyl.

In embodiments of formula (I), X is C₆ to C₃₀ monounsaturated alkenyl. In embodiments, X is C₈ to C₂₈ monounsaturated alkenyl. In embodiments, X is C₁₀ to C₂₆ monounsaturated alkenyl. In embodiments, X is C₁₂ to C₂₆ monounsaturated alkenyl. In embodiments, X is C₁₄ to C₂₄ monounsaturated alkenyl. In embodiments, X is C₁₄ monounsaturated alkenyl. In embodiments, X is C₁₆ monounsaturated alkenyl. In embodiments, X is C₂₀ monounsaturated alkenyl.

In embodiments of formula (I), X is C₆ to C₃₀ polyunsaturated alkenyl. In embodiments, X is C₈ to C₂₈ polyunsaturated alkenyl. In embodiments, X is C₁₀ to C₂₆ polyunsaturated alkenyl. In embodiments, X is C₁₂ to C₂₆ polyunsaturated alkenyl. In embodiments, X is C₁₄ to C₂₄ polyunsaturated alkenyl. In embodiments, X is C₁₄ polyunsaturated alkenyl. In embodiments, X is C₁₆ polyunsaturated alkenyl. In embodiments, X is C₂₀ polyunsaturated alkenyl.

In embodiments of formula (I), A is a conjugation group where A is C═O. In certain embodiments, A is a conjugation group chosen from the following:

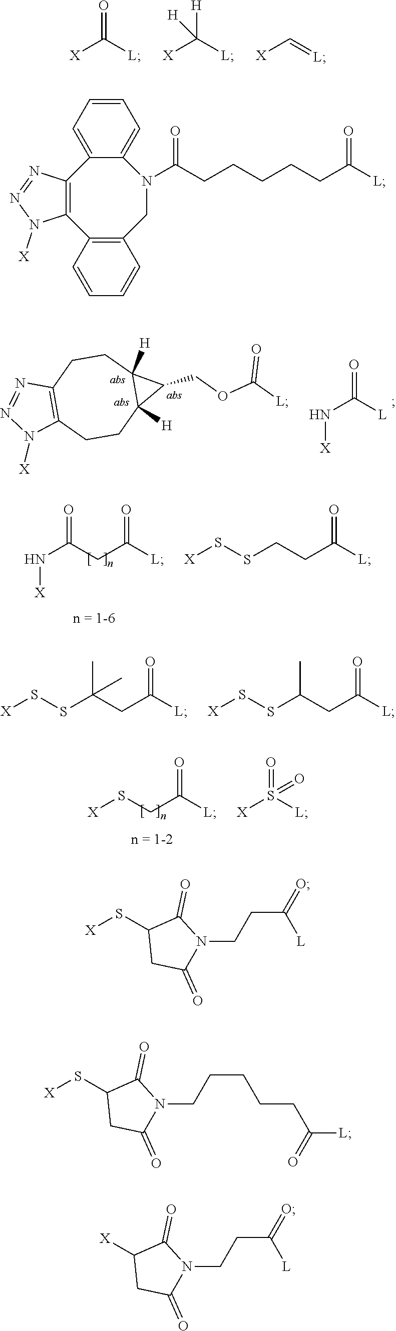

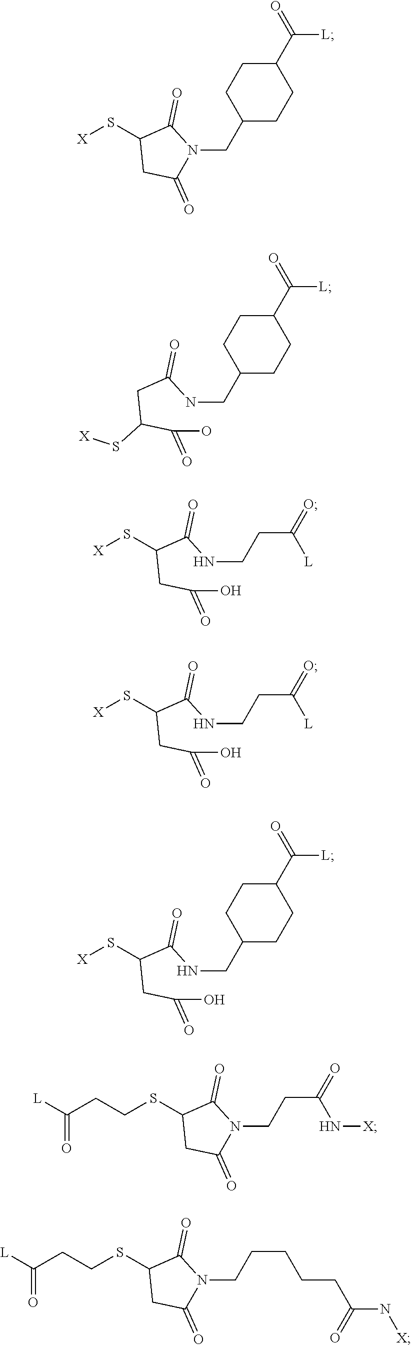

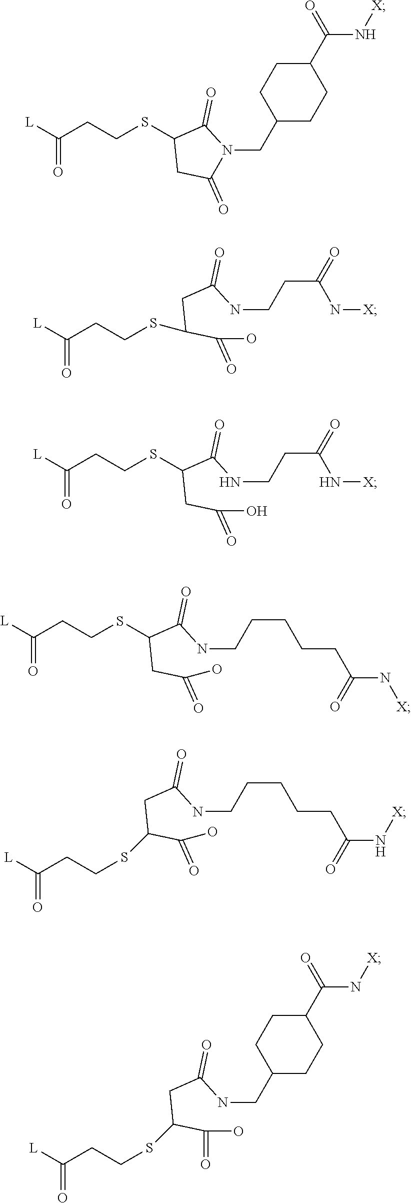

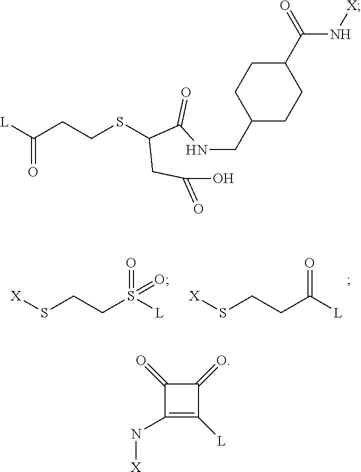

In some embodiments, the conjugation group A contains at least one spacer between the conjugation group A and X in the formula (I) above (e.g., a polyethylene glycol (PEG) chain (e.g., molecular weight ranging from 100 to 2000 Da) and/or at least one amino acid (e.g., cysteine, glutamic acid, lysine, glycine, etc.). Non-limiting examples include:

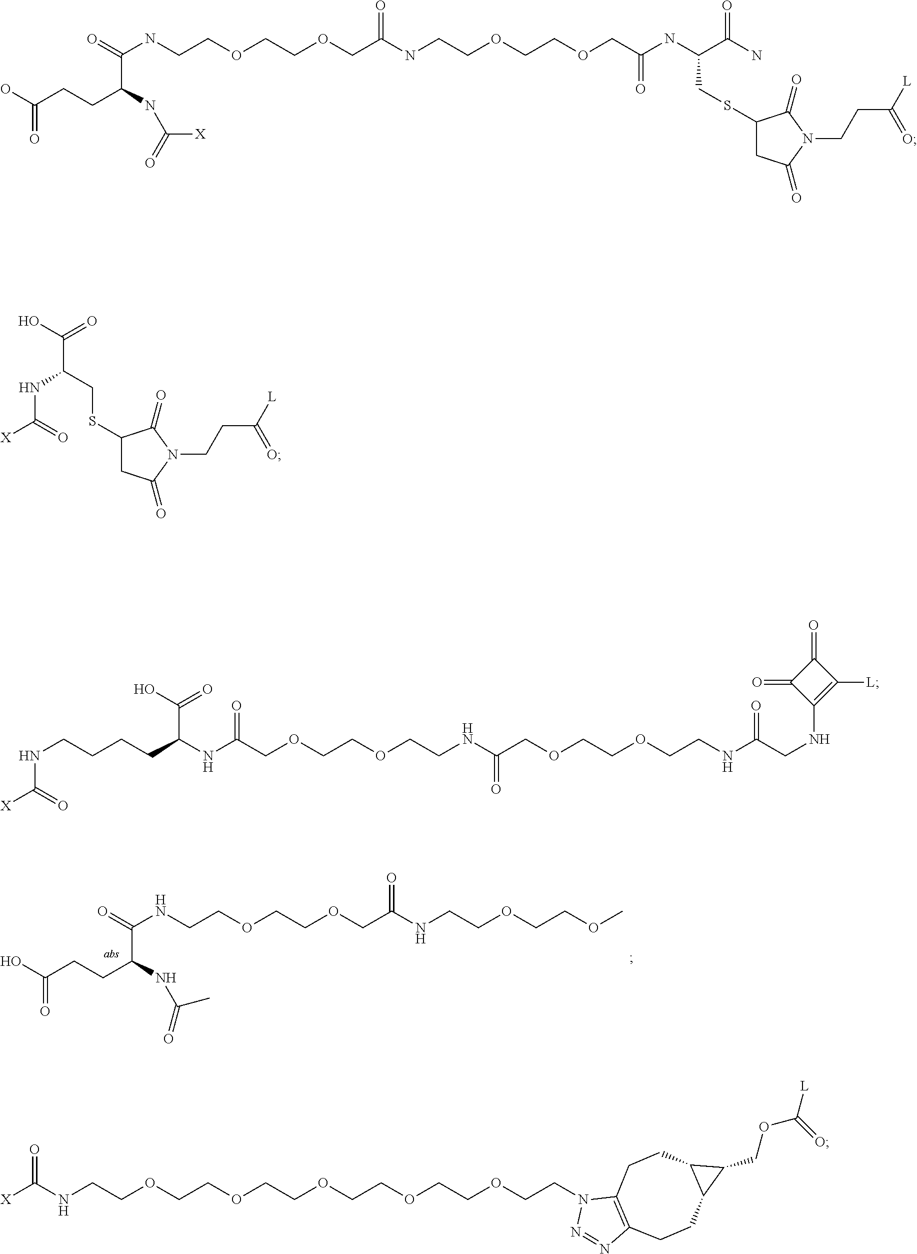

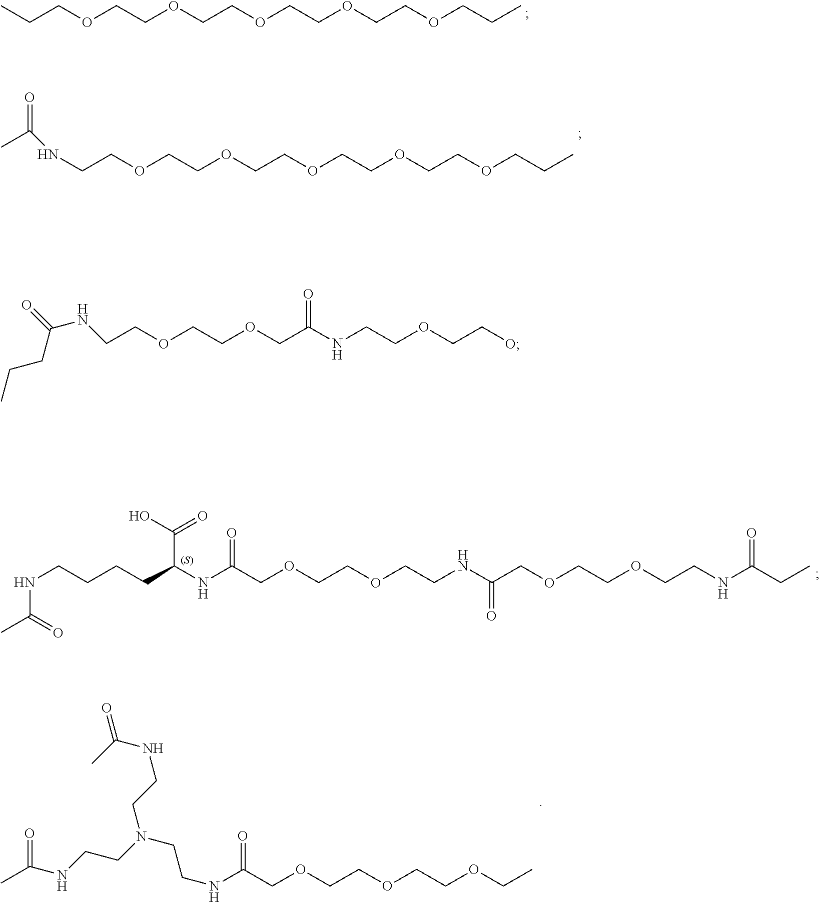

In some embodiments, the conjugation group A contains at least one other fatty acid chain other than the fatty acid chain of formula (I). For example, the conjugation group A contains two fatty acid chains such as illustrated below:

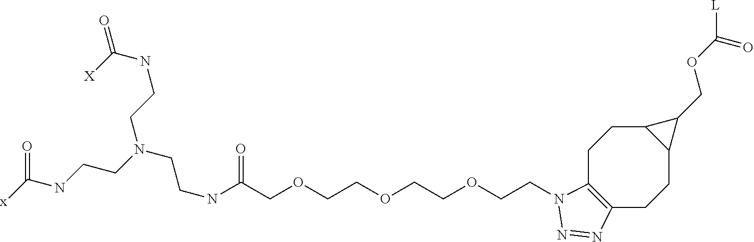

wherein in each instance, X is C₄ to C₃₂ alkyl or alkenyl.

In embodiments of formula (I), L is a linker:

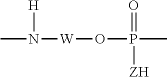

where Z and W are defined below.

In embodiments of formula (I), Z is O or S.

In embodiments of formula (I), W is C₁ to C₁₀ alkyl or alkenyl, or in some embodiments, W is

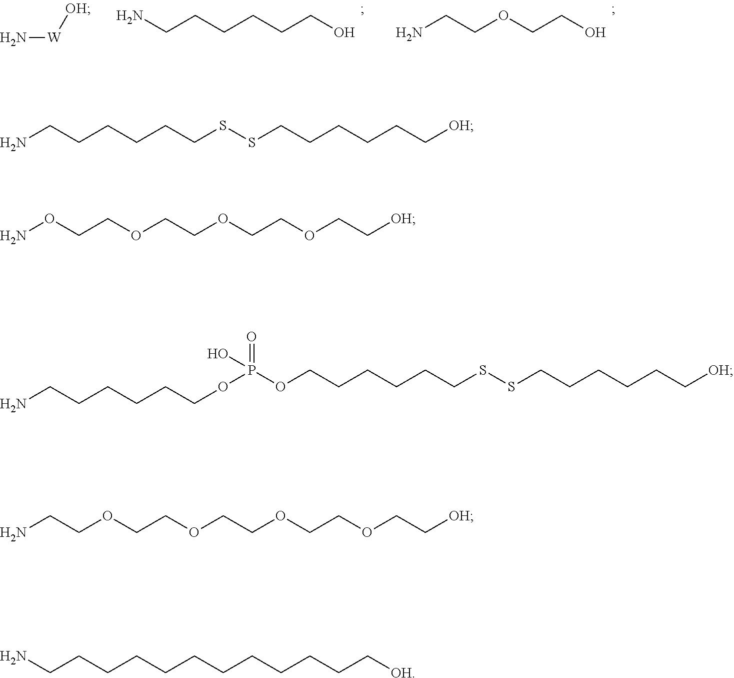

In embodiments, W is C₂ to C₉ alkyl, such as C₃ to C₈ alkyl, for example, W is C₄ to C₈ alkyl. In some embodiments, W is C₅ to C₇ alkyl.

In embodiments, W is C₂ to C₉ monounsaturated alkenyl, for instance W is C₃ to C₈ monounsaturated alkenyl. In some embodiments, W is C₄ to C₈ monounsaturated alkenyl, such as W is C₅ to C₇ monounsaturated alkenyl.

In embodiments, W is C₂ to C₉ polyunsaturated alkenyl, for example W is C₃ to C₈ polyunsaturated alkenyl. In some embodiments, W is C₄ to C₈ polyunsaturated alkenyl, such as W is C₅ to C₇ polyunsaturated alkenyl.

In embodiments, W is C₁ alkyl. In embodiments, W is C₂ alkyl. In embodiments, W is C₃ alkyl. In embodiments, W is C₄ alkyl. In embodiments, W is C₅ alkyl. In embodiments, W is C₆ alkyl. In embodiments, W is C₇ alkyl. In embodiments, W is C₈ alkyl. In embodiments, W is C₉ alkyl. In embodiments, W is C₁₀ alkyl.

In embodiments, W is C₂ monounsaturated alkenyl. In embodiments, W is C₃ monounsaturated alkenyl. In embodiments, W is C₄ monounsaturated alkenyl. In embodiments, W is C₅ monounsaturated alkenyl. In embodiments, W is C₆ monounsaturated alkenyl. In embodiments, W is C₇ monounsaturated alkenyl. In embodiments, W is C₈ monounsaturated alkenyl. In embodiments, W is C₉ monounsaturated alkenyl. In embodiments, W is C₁₀ monounsaturated alkenyl.

In embodiments, W is C₂ polyunsaturated alkenyl. In embodiments, W is C₃ polyunsaturated alkenyl. In embodiments, W is C₄ polyunsaturated alkenyl. In embodiments, W is C₅ polyunsaturated alkenyl. In embodiments, W is C₆ polyunsaturated alkenyl. In embodiments, W is C₇ polyunsaturated alkenyl. In embodiments, W is C₈ polyunsaturated alkenyl. In embodiments, W is C₉ polyunsaturated alkenyl. In embodiments, W is C₁₀ polyunsaturated alkenyl.

In embodiments, L is —NH—CH₂—O—PO₂—. In embodiments, L is —NH—C₂H₄—O—PO₂—. In embodiments, L is —NH—C₃H₆—O—PO₂—. In embodiments, L is —NH—C₄H₈—O—PO₂—. In embodiments, L is —NH—C₅H₁₀—O—PO₂—. In embodiments, L is —NH—C₆H₁₂—O—PO₂—. In embodiments, L is —NH—C₇H₁₄—O—PO₂—. In embodiments, L is —NH—C₈H₁₆—O—PO₂—. In embodiments, L is —NH—C₉H₁₈—O—PO₂—. In embodiments, L is —NH—C₁₀H₂₀—O—PO₂—.

In embodiments, L is attached to the 5′ end of the oligonucleotide Y. In embodiments, the phosphate group in L is a 5′ phosphate group on the oligonucleotide. In embodiments, L is attached to the 3′ end of the oligonucleotide Y. In embodiments, L is attached to the oligonucleotide at a modified base in the oligonucleotide Y.

In embodiments, the present disclosure provides an acid acyl conjugated oligonucleotide of formula (I)

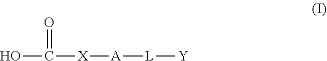

wherein: X is C₁₀ to C₂₆ alkyl; A is a conjugation group chosen from the groups described above; L is —NH—C₆H₁₂—O—PO₂—; and Y is an oligonucleotide as described herein attached to L at the 5′ end of Y.

In embodiments, the present disclosure provides an acid acyl conjugated oligonucleotide of formula (I)

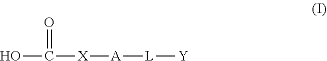

wherein: X is chosen from C₁₄ alkyl, C₁₆ alkyl, and C₂₀ alkyl; A is a conjugation group chosen from the groups described above; L is —NH—C₆H₁₂—O—PO₂—; and Y is an oligonucleotide as described herein attached to L at the 5′ end of Y.

In embodiments, the present disclosure provides an acid acyl conjugated oligonucleotide of formula (Ia)

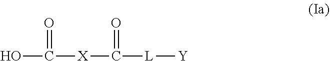

wherein: X is C₁₄ alkyl; L is —NH—C₆H₁₂—O—PO₂—; and Y is an oligonucleotide as described herein attached to L at the 5′ end of Y.

In embodiments, the present disclosure provides an acid acyl conjugated oligonucleotide of formula (Ia)

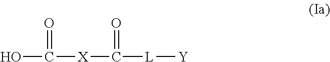

wherein: X is C₁₆ alkyl; L is —NH—C₆H₁₂—O—PO₂—; and Y is an oligonucleotide as described herein attached to L at the 5′ end of Y.

In embodiments, the present disclosure provides an acid acyl conjugated oligonucleotide of formula (Ia)

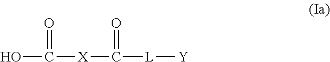

wherein: X is C₂₀ alkyl; L is —NH—C₆H₁₂—O—PO₂—; and Y is an oligonucleotide as described herein attached to L at the 5′ end of Y.

In any of the above embodiments of formula (I), the oligonucleotide Y can be any oligonucleotide described herein.

### Oligonucleotides for Conjugation and Methods of Treatment

In any embodiment of the present disclosure, the oligonucleotide (Y) of the compounds described herein can comprise DNA, RNA or nucleic acids having unnatural backbones. The oligonucleotide can comprise the natural DNA and RNA nucleobases: adenine (A), cytosine (C), guanine (G), thymine (T), and uracil (U), and may also comprise non-natural and modified nucleobases. The oligonucleotide can have a non-natural backbone such as a phosphorothioate backbone. In some embodiments, the oligonucleotide is single stranded. In some embodiments, the oligonucleotide is double stranded.

In embodiments, the oligonucleotide comprises an antisense oligonucleotide. In embodiments, the oligonucleotide comprises a sequence that can be expressed in a cell, e.g., a coding sequence such as a gene or a messenger RNA (mRNA). In embodiments, the oligonucleotide comprises a sequence that does not encode a protein but has another function in the cell, e.g., a non-coding RNA, a transfer RNA (tRNA), a ribosomal RNA (rRNA) or a small-nucleolar RNA (snRNA). In embodiments, the oligonucleotide is a CRISPR guide RNA (gRNA).

In embodiments, the oligonucleotide is an antisense oligonucleotide targeting MALAT1, apolipoprotein B, apolipoprotein C III, endothelial lipase, p53, clusterin, signal transducer and activator of transcription 3 (STAT3), Mothers against decapentaplegic homolog 7 (SMAD7), intercellular adhesion molecule 1 (CD54), dystrophin (for example, myotonic dystrophy protein kinase (DMPK), transthyretin (TTR), huntingtin (HTT), microRNA-122 (miR-122). In embodiments, the oligonucleotide is an antisense oligonucleotide that interferes with splicing of dystrophin mRNA such as eteplirsen, golodirsen, casimersen, drisapersen and viltolarsen). In embodiments, the oligonucleotide is an antisense oligonucleotide that reduces expression of any of the above targets. In embodiments, the oligonucleotide is an antisense oligonucleotide that reduces mRNA splicing of any of the above targets. In embodiments, the oligonucleotide is an antisense oligonucleotide that targets a non-coding sequence in order to reduce expression of any of the above targets, e.g., an exon, a 5′ non-coding sequence or a 3′ non-coding sequence.

In some embodiments the oligonucleotide comprises a sequence that can be expressed in a cell, such as a protein expressed by a gene, the lipid conjugated oligonucleotide can be used in methods of treating subjects that lack sufficient expression of the protein, e.g., in gene therapy type methods of treatment. In embodiments where the oligonucleotide comprises a sequence that does not encode a protein but has another function, the lipid conjugated oligonucleotide can be used in methods of treating subjects that lack the function provided by the oligonucleotide.

In embodiments where the oligonucleotide is antisense to a target, the lipid conjugated oligonucleotide can be used in methods of treating subjects having disorders relating to increased levels of expression of the target. In embodiments where the oligonucleotide is antisense to a target, the oligonucleotide reduces expression of the target in cells by about 1% to about 100%. In embodiments, the oligonucleotide reduces expression of the target in cells by about 1%, by about 10%, by about 20%, by about 30%, by about 40%, by about 50%, by about 60%, by about 70%, by about 80%, by about 90%, by about 95% or by about 100%. In embodiments, the oligonucleotide reduces expression of the target in cells by about 1% to about 100%, by about 5% to about 50%, by about 10% to about 50%, by about 30%, by about 10% to about 30%, or by about 15% to about 25%.

In some embodiments, the oligonucleotide is an antisense oligonucleotide targeting a condition effecting the heart, as the carboxy acyl conjugate can be used to preferentially target to the oligonucleotide to cardiac tissue. In embodiments where the oligonucleotide is antisense to a target, the oligonucleotide reduces expression of the target in cardiac cells by about 1% to about 100%. In embodiments, the oligonucleotide reduces expression of the target in cardiac cells by about 1%, by about 10%, by about 20%, by about 30%, by about 40%, by about 50%, by about 60%, by about 70%, by about 80%, by about 90%, by about 95% or by about 100%. In embodiments, the oligonucleotide reduces expression of the target in cardiac cells by about 1% to about 100%, by about 5% to about 50%, by about 10% to about 50%, by about 30%, by about 10% to about 30%, or by about 15% to about 25%.

In embodiments, the oligonucleotide is an antisense oligonucleotide targeting metastasis-associated lung adenocarcinoma transcript 1 (MALAT1). In embodiments, the oligonucleotide is an antisense oligonucleotide targeting MALAT1, variant 1 (SEQ ID NO: 1). In embodiments, the oligonucleotide is an antisense oligonucleotide targeting MALAT1, variant 2 (SEQ ID NO:2). In embodiments, the oligonucleotide is an antisense oligonucleotide targeting MALAT1, variant 3 (SEQ ID NO:3). In embodiments, the antisense oligonucleotide targets MALAT1 and has the sequence: tcagcattctaatagcagc (SEQ ID NO:4). In embodiments, the antisense oligonucleotide targets MALAT1 and has the sequence: tm5cagm5cattm5ctaatagm5cagm5c, where m5c is 5-methylcytidine (SEQ ID NO:5). In embodiments, the antisense oligonucleotide targets MALAT1 and has the sequence: gcattctaatagcagc (SEQ ID NO:6). In embodiments, the antisense oligonucleotide targets MALAT1 and has the sequence: gm5cattm5ctaatagm5cagm5c, where m5c is 5-methylcytidine (SEQ ID NO:7). In embodiments, the oligonucleotide is an antisense oligonucleotide that targets MALAT1 and reduces expression of MALAT1 in cells by about 1% to about 100%. In embodiments, the oligonucleotide is an antisense oligonucleotide that targets MALAT1 and reduces expression of MALAT1 in cells by about 1%, by about 10%, by about 20%, by about 30%, by about 40%, by about 50%, by about 60%, by about 70%, by about 80%, by about 90%, by about 95% or by about 100%.

In embodiments, the oligonucleotide is an antisense oligonucleotide targeting metastasis-associated lung adenocarcinoma transcript 1 (MALAT1) comprising at least one nucleic acid with a locked sugar modified moiety (“LNA”). In such embodiments, the antisense oligonucleotide targets MALAT1 and has the sequence: GM5CAttm5ctaatagm5cAGM5C, where m5c is 5-methylcytidine and capital letters are LNA nucleosides (SEQ ID NO:8). In embodiments, the oligonucleotide is an antisense oligonucleotide that targets MALAT1 and reduces expression of MALAT1 in cells by about 1% to about 100%. In embodiments, the oligonucleotide is an antisense oligonucleotide that targets MALAT1 and reduces expression of MALAT1 in cells by about 1%, by about 10%, by about 20%, by about 30%, by about 40%, by about 50%, by about 60%, by about 70%, by about 80%, by about 90%, by about 95% or by about 100%.

In embodiments, the oligonucleotide is an antisense oligonucleotide targeting Calcium/Calmodulin Dependent Protein Kinase II Delta (CAMK2D). In embodiments, the antisense oligonucleotide targeting CAMK2D comprises at least one nucleic acid with a locked sugar modified moiety (“LNA”). In some embodiments, the antisense oligonucleotide targets CAMK2D and has the sequence: GTTtggtattm5cttTAG, where m5c is 5-methylcytidine and capital letters are LNA nucleosides (SEQ ID NO:9). In some embodiments, the antisense oligonucleotide targets CAMK2D and has the sequence: GTGtm5caam5caam5cm5caTTT, where m5c is 5-methylcytidine and capital letters are LNA nucleosides (SEQ ID NO:10). In some embodiments, the antisense oligonucleotide targets CAMK2D and has the sequence: M5CAM5CAaatttattaaM5CTM5CT, where m5c is 5-methylcytidine and capital letters are LNA nucleosides (SEQ ID NO: 11). In some embodiments, the antisense oligonucleotide targets CAMK2D and has the sequence: M5CTGttm5cttm5caAtaATG, where m5c is 5-methylcytidine and capital letters are LNA nucleosides (SEQ ID NO: 12). In some embodiments, the antisense oligonucleotide targets CAMK2D and has the sequence: AM5CM5Catgagm5ctataM5CTT, where m5c is 5-methylcytidine and capital letters are LNA nucleosides (SEQ ID NO:13). In embodiments, the oligonucleotide is an antisense oligonucleotide that targets CAMK2D and reduces expression of CAMK2D in cells by about 1% to about 100%. In embodiments, the oligonucleotide is an antisense oligonucleotide that targets CAMK2D and reduces expression of CAMK2D in cells by about 1%, by about 10%, by about 20%, by about 30%, by about 40%, by about 50%, by about 60%, by about 70%, by about 80%, by about 90%, by about 95% or by about 100%.

In embodiments, the oligonucleotide is an antisense oligonucleotide that targets MALAT1 and reduces expression of MALAT1 in cardiac cells by about 1% to about 100%. In embodiments, the oligonucleotide is an antisense oligonucleotide that targets MALAT1 and reduces expression of MALAT1 in cardiac cells by about 1%, by about 10%, by about 20%, by about 30%, by about 40%, by about 50%, by about 60%, by about 70%, by about 80%, by about 90%, by about 95% or by about 100%.

In embodiments where the oligonucleotide is an antisense oligonucleotide that targets MALAT1, the lipid conjugated oligonucleotide can be used in a method of treatment of a cardiac disease in a subject. In embodiments where the oligonucleotide is an antisense oligonucleotide that targets MALAT1, the lipid conjugated oligonucleotide can be used in a method of treatment of a myocardial infarction in a subject. In embodiments where the oligonucleotide is an antisense oligonucleotide that targets MALAT1, the lipid conjugated oligonucleotide can be used in a method of preventing a myocardial infarction in a subject. In embodiments where the oligonucleotide is an antisense oligonucleotide that targets MALAT1, the oligonucleotide is administered to a subject at risk for a myocardial infarction.

### Methods of Making Lipid Conjugated Oligonucleotides

In embodiments, the present disclosure provides methods of making the lipid conjugated oligonucleotides described herein. In embodiments, the present disclosure provides a method of making an acid acyl oligonucleotide of formula (Ia):

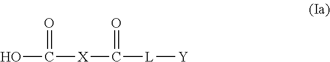

wherein: X is C₄ to C₃₂ alkyl or alkenyl; L is a linker; and Y is an oligonucleotide.

In embodiments, the method of making formula (I) comprises the following steps:

A) providing a fatty diacid of formula (II)

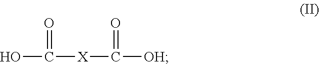

B) reacting the fatty diacid with an activating group, A, to form an acid acyl compound of formula (III):

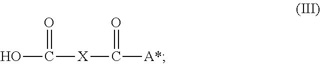

wherein A* is the activating group attached to the fatty acyl compound; and
C) reacting the compound of formula (III) with a compound of formula (IV):

L-Y (IV),

wherein *L is a linker with a reactive group;
to form the compound of formula (Ia):

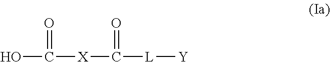

wherein: X is C₄ to C₃₂ alkyl or alkenyl; L is a linker; and Y is an oligonucleotide.

In embodiments of the method of making formula (I), the activating group A* is an activated ester amine. In embodiments, the activating group A* in step B) may be chosen from:

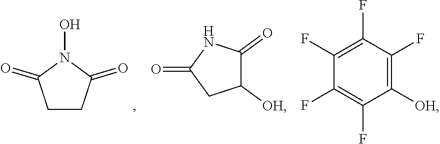

### TBTU, and HATU.

In embodiments of the method of making formula (I), the reaction in step B) requires reacting a carboxylic acid with an amine to form an activated ester amine. In embodiments, the reaction in step B) of the method of making requires reacting a carboxylic acid with N-hydroxysuccinimide (NHS) to form an NHS ester as an activated ester amine.

In embodiments, the reaction in step B) of the method of making is performed in an organic solvent. In embodiments, the reaction in step B) of the method of making is performed in a solvent comprising one or more of ethyl acetate, dioxane, tetrahydrofuran, dimethylfuran and dichloromethane and mixtures thereof.

In embodiments, the reaction in step B) of the method of making is performed in the presence of a coupling reagent. In embodiments, the coupling reagent is a diimine. In embodiments, the coupling reagent is dicyclohexylmethanediimine.

In embodiments of the method of making formula (I), the reaction in step C) requires coupling between the activating group A* attached to the fatty acyl compound and a primary amine on the linker (L).

In embodiments of the method of making formula (I), the compound of formula (IV)

*L-Y (IV)

is dissolved in an aqueous buffer. In embodiments, the compound of formula (IV) is dissolved in a phosphate buffer, a borate buffer, a carbonate buffer, an acetate buffer, a Tris buffer, a HEPES buffer, a MOPS buffer or a PIPES buffer, or is dissolved in pure water with a base such as triethylamine or DIPEA.

In embodiments of the method of making formula (I), the compound of formula (III)

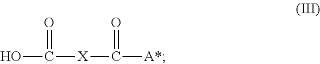

is dissolved in a polar aprotic solvent. In embodiments of the method of making formula (I), the compound of formula (III) is dissolved in acetonitrile. In other embodiments of the method of making formula (I), the compound of formula (III) is dissolved in dimethyl sulfoxide. In other embodiments of the method of making formula (I), the compound of formula (III) is dissolved in a mixture of acetonitrile and dimethyl sulfoxide.

In other embodiments, the method of making formula (I) comprises the following steps:

A) providing a fatty acid of formula (IIa)

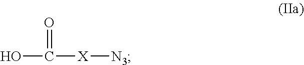

B) reacting the compound of formula (IIa) with a compound of formula (IV):

*L-Y (IV),

wherein *L is a linker with a reactive group;
to form the compound of formula (I):

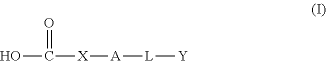

wherein: X is C₄ to C₃₂ alkyl or alkenyl; A is a conjugation group; L is a linker; and Y is an oligonucleotide as described above.

In embodiments of the method of making formula (I), the linker with a reactive group L* is

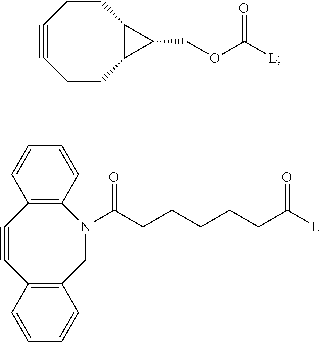

In embodiments of the method of making formula (I), the resultant product of either step B) and/or step C) can be purified. In embodiments, the resultant product of either step B) and/or step C) is purified by chromatography. In embodiments, the resultant product of either step B) and/or step C) is purified by flash chromatography.

In embodiments of any of the above methods for making formula (I), the length of the acyl group, X, is C₆ to C₃₀ alkyl or alkenyl. In embodiments, X is C₈ to C₂₈ alkyl or alkenyl. In embodiments, X is C₁₀ to C₂₆ alkyl or alkenyl. In embodiments, X is C₁₂ to C₂₆ alkyl or alkenyl. In embodiments, X is C₁₄ to C₂₄ alkyl or alkenyl. In embodiments, X is C₁₄ alkyl or alkenyl. In embodiments, X is C₁₆ alkyl or alkenyl. In embodiments, X is C₂₀ alkyl or alkenyl.

In embodiments of any of the above methods for making formula (I), the length of the acyl group, X, is C₆ to C₃₀ alkyl. In embodiments, X is C₈ to C₂₈ alkyl. In embodiments, X is C₁₀ to C₂₆ alkyl. In embodiments, X is C₁₂ to C₂₆ alkyl. In embodiments, X is C₁₄ to C₂₄ alkyl. In embodiments, X is C₁₄ alkyl. In embodiments, X is C₁₆ alkyl. In embodiments, X is C₂₀ alkyl.

In embodiments of any of the above methods for making formula (I), X is C₆ to C₃₀ monounsaturated alkenyl. In embodiments, X is C₈ to C₂₈ monounsaturated alkenyl. In embodiments, X is C₁₀ to C₂₆ monounsaturated alkenyl. In embodiments, X is C₁₂ to C₂₆ monounsaturated alkenyl. In embodiments, X is C₁₄ to C₂₄ monounsaturated alkenyl. In embodiments, X is C₁₄ monounsaturated alkenyl. In embodiments, X is C₁₆ monounsaturated alkenyl. In embodiments, X is C₂₀ monounsaturated alkenyl.

In any of the above methods for making formula (I), X is C₆ to C₃₀ polyunsaturated alkenyl. In embodiments, X is C₈ to C₂₈ polyunsaturated alkenyl. In embodiments, X is C₁₀ to C₂₆ polyunsaturated alkenyl. In embodiments, X is C₁₂ to C₂₆ polyunsaturated alkenyl. In embodiments, X is C₁₄ to C₂₄ polyunsaturated alkenyl. In embodiments, X is C₁₄ polyunsaturated alkenyl. In embodiments, X is C₁₆ polyunsaturated alkenyl. In embodiments, X is C₂₀ polyunsaturated alkenyl.

In embodiments of any of the above methods for making formula (I), the linker (L) comprises a C₃-C₁₀ amine. In embodiments of the method of making formula (I), the linker (L) comprises a C₄-C₉ amine. In embodiments of the method of making formula (I), the linker (L) comprises a C₅-C₈ amine. In embodiments of the method of making formula (I), the linker (L) comprises a C₆ or C₇ amine. In embodiments of the method of making formula (I), the linker (L) comprises a C₆ amine (hexylamine).

In embodiments, the linker (L) forms an amide bond with the carboxy acyl group. In embodiments, the linker (L) comprises a phosphate terminal connected to the oligonucleotide. In some embodiments, the linker (L) is

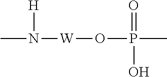

wherein W is C₁ to C₁₀ alkyl or alkenyl.

In embodiments, W is C₂ to C₉ alkyl. In embodiments, W is C₃ to C₈ alkyl. In embodiments, W is C₄ to C₈ alkyl. In embodiments, W is C₅ to C₇ alkyl.

In embodiments, W is C₂ to C₉ monounsaturated alkenyl. In embodiments, W is C₃ to C₈ monounsaturated alkenyl. In embodiments, W is C₄ to C₈ monounsaturated alkenyl. In embodiments, W is C₅ to C₇ monounsaturated alkenyl.

In embodiments, W is C₂ to C₉ polyunsaturated alkenyl. In embodiments, W is C₃ to C₈ polyunsaturated alkenyl. In embodiments, W is C₄ to C₈ polyunsaturated alkenyl. In embodiments, W is C₅ to C₇ polyunsaturated alkenyl.

In embodiments, W is C₁ alkyl. In embodiments, W is C₂ alkyl. In embodiments, W is C₃ alkyl. In embodiments, W is C₄ alkyl. In embodiments, W is C₅ alkyl. In embodiments, W is C₆ alkyl. In embodiments, W is C₇ alkyl. In embodiments, W is C₈ alkyl. In embodiments, W is C₉ alkyl. In embodiments, W is C₁₀ alkyl.

In embodiments, W is C₂ monounsaturated alkenyl. In embodiments, W is C₃ monounsaturated alkenyl. In embodiments, W is C₄ monounsaturated alkenyl. In embodiments, W is C₅ monounsaturated alkenyl. In embodiments, W is C₆ monounsaturated alkenyl. In embodiments, W is C₇ monounsaturated alkenyl. In embodiments, W is C₈ monounsaturated alkenyl. In embodiments, W is C₉ monounsaturated alkenyl. In embodiments, W is C₁₀ monounsaturated alkenyl.

In embodiments, W is C₂ polyunsaturated alkenyl. In embodiments, W is C₃ polyunsaturated alkenyl. In embodiments, W is C₄ polyunsaturated alkenyl. In embodiments, W is C₅ polyunsaturated alkenyl. In embodiments, W is C₆ polyunsaturated alkenyl. In embodiments, W is C₇ polyunsaturated alkenyl. In embodiments, W is C₈ polyunsaturated alkenyl. In embodiments, W is C₉ polyunsaturated alkenyl. In embodiments, W is C₁₀ polyunsaturated alkenyl.

In embodiments, the linker (L) is —NH—CH₂—O—PO₂—. In embodiments, L is —NH—C₃H₆—O—PO₂—. In embodiments, L is —NH—C₄H₈—O—PO₂—. In embodiments, L is —NH—C₅H₁₀—O—PO₂—. In embodiments, L is —NH—C₆H₁₂—O—PO₂—. In embodiments, L is —NH—C₇H₁₄—O—PO₂—. In embodiments, L is —NH—C₈H₁₆—O—PO₂—. In embodiments, L is —NH—C₉H₁₈—O—PO₂—. In embodiments, L is —NH—C₁₀H₂₀—O—PO₂—.

In embodiments of any of the above methods for making formula (I), L is attached to the 5′ end of the oligonucleotide Y. In embodiments, L is attached to the 3′ end of the oligonucleotide Y. In embodiments, L is attached to the oligonucleotide at a modified base in the oligonucleotide Y.

In embodiments of any of the above methods for making formula (I), the oligonucleotide can be unmodified DNA, RNA or may be modified. Modified oligonucleotides comprise at least one modification relative to unmodified RNA or DNA (i.e., comprise at least one modified nucleoside (comprising a modified sugar moiety and/or a modified nucleobase and/or at least one modified internucleoside linkage). In embodiments, the oligonucleotide can be selected from any of the oligonucleotides described herein. In embodiments, the oligonucleotide is an antisense oligonucleotide. In embodiments, the antisense oligonucleotide contains at least one phosphorothioate internucleoside linkage. In embodiments, the antisense oligonucleotide contains at least one modified sugar moiety, for example, a bicyclic sugar moiety (e.g., comprising two rings, wherein the second ring is formed via a bridge connecting two of the atoms in the first ring thereby forming a bicyclic structure). In embodiments, the antisense oligonucleotide contains at least one modified nucleobase.

### Methods of Delivery and Use of Lipid Conjugated Oligonucleotides

In embodiments, the lipid conjugated oligonucleotides are preferentially delivered to a specific tissue in the body upon administration. In embodiments, the lipid conjugated oligonucleotides are, for instance, delivered to cardiac tissue upon administration. In embodiments, the lipid conjugated oligonucleotides are, for example, delivered to liver tissue upon administration. In embodiments, the lipid conjugated oligonucleotides are, for instance, delivered to spleen tissue upon administration. In embodiments, the lipid conjugated oligonucleotides are, for example, delivered to kidney tissue upon administration. In embodiments, the lipid conjugated oligonucleotides are, for example, delivered to skin tissue upon administration. In embodiments, the lipid conjugated oligonucleotides are, for example, delivered to muscle tissue upon administration. In embodiments, the lipid conjugated oligonucleotides are, for example, delivered to lung tissue upon administration. In embodiments, the lipid conjugated oligonucleotides are, for example, delivered to adipose tissue upon administration.

In embodiments, the present disclosure provides a method of delivering an oligonucleotide to cardiac tissue in a subject comprising: a) providing an acid acyl conjugated oligonucleotide as described herein, and b) administering the acid acyl conjugated oligonucleotide to the subject.

In embodiments, the present disclosure provides a method of reducing expression of a gene of interest in the cardiac cells of a subject, comprising a) providing an acid acyl conjugated oligonucleotide as described herein, and b) administering the acid acyl conjugated oligonucleotide to the subject.

In embodiments of the method of reducing expression of a gene of interest in the cardiac cells of a subject, the gene of interest is any antisense target described herein. In embodiments of the method of reducing expression of a gene of interest in the cardiac cells of a subject, the gene of interest is MALAT1.

In embodiments of the methods herein, the oligonucleotide can be DNA, RNA or a modified oligonucleotide. In embodiments, the oligonucleotide can be selected from any of the oligonucleotides described herein. In embodiments, the oligonucleotide is an antisense oligonucleotide. In embodiments, the oligonucleotide has a phosphorothioate backbone.

In embodiments of the methods herein, the subject is a mammal. In embodiments of the methods herein, the mammalian subject is an animal such as an agricultural animal (e.g., cattle, sheep, swine), research animal (e.g., mice, rats, monkeys, chimpanzees) or companion animal (e.g., dogs, cats and rabbits). In embodiments of the methods herein, the mammalian subject is a human.

In embodiments of the methods herein, the oligonucleotide is administered to treat a cardiac disease. In embodiments of the methods herein, the oligonucleotide is administered to treat a myocardial infarction. In embodiments of the methods herein, the oligonucleotide is administered to prevent a myocardial infarction. In embodiments of the method of delivering an oligonucleotide to cardiac tissue in a subject, the oligonucleotide is administered to a subject at risk for a myocardial infarction.

### Pharmaceutical Formulations

In embodiments, the lipid conjugated oligonucleotides described herein are formulated into a pharmaceutically acceptable formulation. In some embodiments, the pharmaceutical formulation further comprises a pharmaceutically acceptable excipient, e.g., tonicity adjusting agent, preservative, solubilizing agent, complexing agent, dispersing agent, buffering agent, or combination thereof. In some embodiments, the pharmaceutical formulation is suitable for administration to a patient. In some embodiments, the pharmaceutical formulation is suitable for intramuscular, subcutaneous, intravenous, intraperitoneal or oral administration to a patient.

### EXAMPLES

### Intermediates

### Activated C22 Acid (Intermediate 1)—22-((2,5-dioxopyrrolidin-1-yl)oxy)-22-oxodocosanoic acid

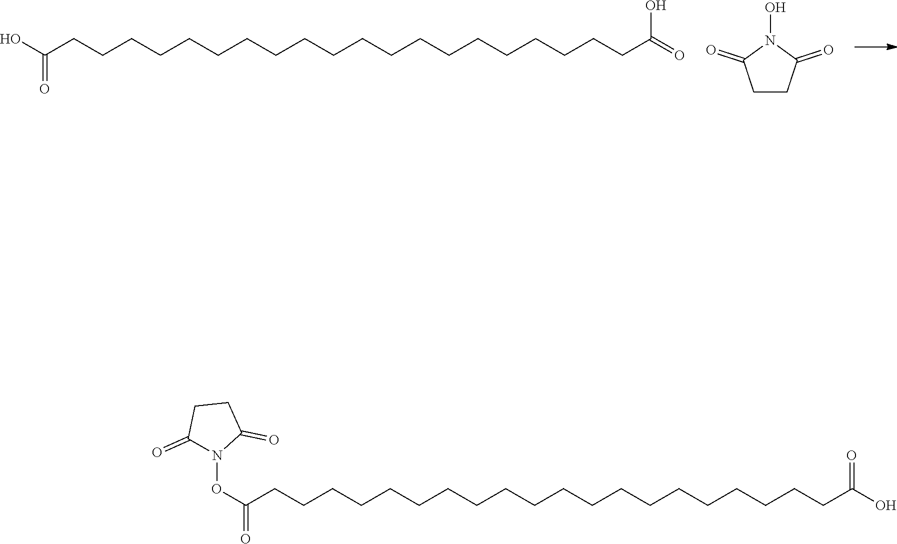

Docosanedioic acid (0.5 g, 1.35 mmol), 1-hydroxypyrrolidine-2,5-dione (0.155 g, 1.35 mmol) and N,N-dimethylpyridin-4-amine (cat. amount) were added to tetrahydrofuran (THF) (17 mL) and stirred at room temperature for 10 min. Dicyclohexylmethanediimine (0.278 g, 1.35 mmol) solvated in THF (6.00 mL) was added dropwise for 30 min. and the mixture was then stirred for 24 h. at room temperature. After filtration, the mixture was evaporated. Methanol (MeOH) (5 ml) was added to the residue, heated to 45° C. and was then stirred for 1 h. at room temperature. The product (22-((2,5-dioxopyrrolidin-1-yl)oxy)-22-oxodocosanoic acid) was crystalized and was separated by filtration, washed with a small amount of MeOH and dried under reduced pressure to give the desired product: 22-((2,5-dioxopyrrolidin-1-yl)oxy)-22-oxodocosanoic acid. Yield was 353 mg (56%). LC/MS and H, NMR are in agreement with the expected product.

MS (ESI) m/z 466.6 [M-H]⁻

¹H NMR (500 MHz, CDCl₃) 1.1-1.46 (33H, m), 1.63 (2H, t), 1.68-1.79 (2H, m), 2.35 (2H, td), 2.60 (2H, t) 2.84 (4H, d),

### NHS Activated C16 Acid (Intermediate 2)—16-((2,5-dioxopyrrolidin-1-yl)oxy)-16-oxohexadecanoic acid

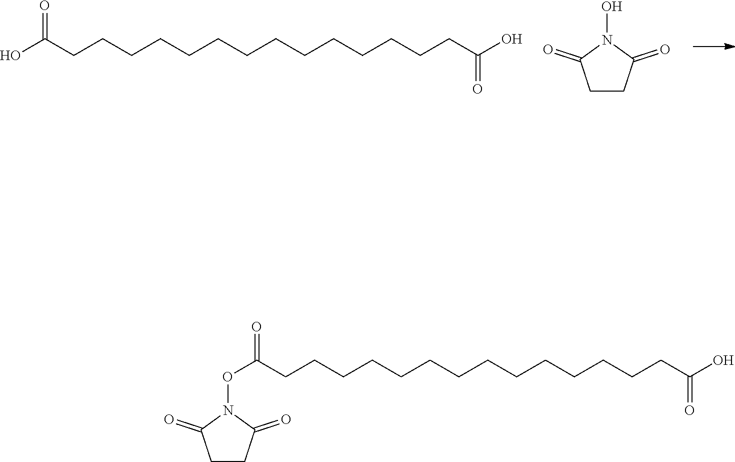

The compound was made according to Intermediate 1 starting from hexadecanedioic acid (0.3 g, 1.05 mmol). After crystallization, the residue was purified with flash chromatography on silica using ethyl acetate/Heptane 1/1 as eluent. The pure fractions was evaporated to give the desired product (16-((2,5-dioxopyrrolidin-1-yl)oxy)-16-oxohexadecanoic acid) Yield: 120 mg (30%).

MS (ESI) m/z 382.0 [M-H]⁻

¹H NMR (500 MHz, CDCl3) 1.28 (21H, d), 1.64 (2H, td), 1.69-1.79 (2H, m), 2.35 (2H, td), 2.60 (2H, t), 2.84 (4H, d).

### NHS Activated C17 Acid (Intermediate 3)—17-((2,5-dioxopyrrolidin-1-yl)oxy)-17-oxoheptadecanoic acid

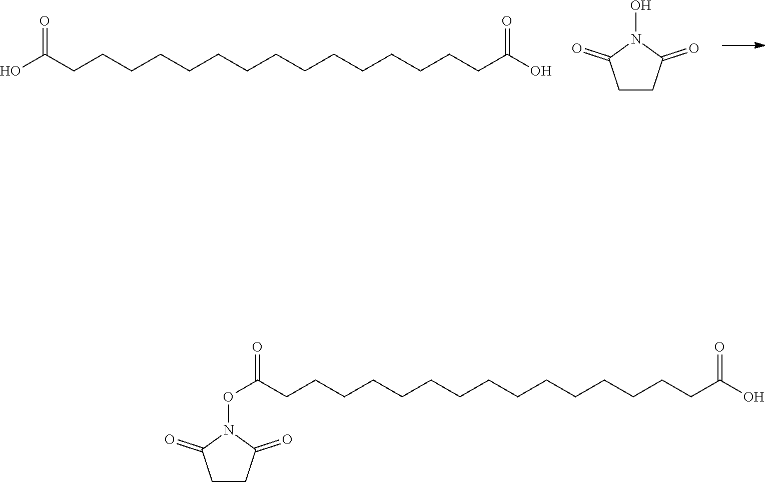 [SMILES: O=C(O)CCCCCCCCCCCCCCCC(=O)O ; O=C1CCC(=O)N1O ; O=C(O)CCCCCCCCCCCCCCCC(=O)ON1C(=O)CCC1=O]

The compound was made according to Intermediate 1 starting from heptadecanedioic acid (0.3 g, 1.0 mmol). After crystallization, the residue was purified with flash chromatography on silica using ethyl acetate/Heptane 1/1 as eluent. The pure fraction was evaporated to give of the desired product (17-((2,5-dioxopyrrolidin-1-yl)oxy)-17-oxoheptadecanoic acid) Yield: 99 mg, 25%.

MS (ESI) m/z 396.2 [M-H]⁻

¹H NMR (500 MHz, CDCl₃) 1.27 (23H, d), 1.55-1.69 (2H, m), 1.74 (2H, p), 2.35 (2H, td), 2.60 (2H, t), 2.76-2.91 (4H, m).

### NHS Activated C18 Acid (Intermediate 4)—18-((2,5-dioxopyrrolidin-1-yl)oxy)-18-oxooctadecanoic acid

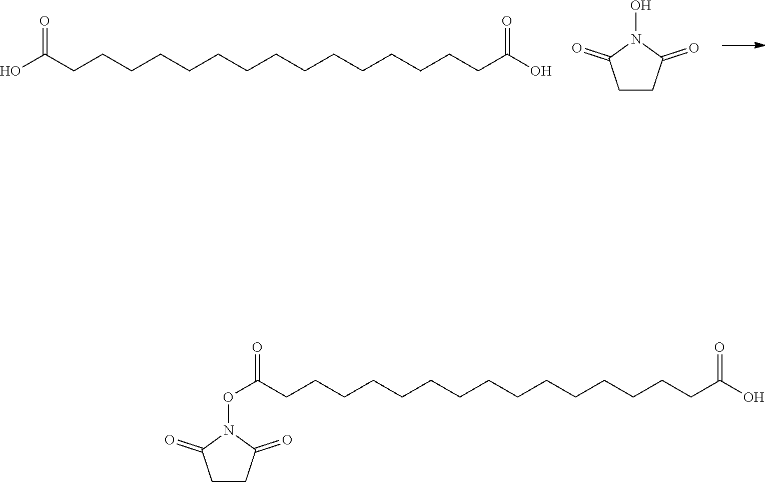 [SMILES: O=C(O)CCCCCCCCCCCCCCCC(=O)O ; O=C1CCC(=O)N1O ; O=C(O)CCCCCCCCCCCCCCCC(=O)ON1C(=O)CCC1=O]

The compound was made according to Intermediate 1 starting from octadecanedioic acid (0.3 g, 0.95 mmol). After crystallization, the residue was purified with flash chromatography on silica using ethyl acetate/Heptane 2/1 as eluent. The pure fraction was evaporated to give of the desired product (18-((2,5-dioxopyrrolidin-1-yl)oxy)-18-oxooctadecanoic acid) Yield: 93 mg, 24%.

MS (ESI) m/z 410.3 [M-H]⁻

¹H NMR (500 MHz, CDCl₃) 1.25 (25H, s), 1.63 (2H, q), 1.74 (2H, p), 2.35 (2H, t), 2.60 (2H, t), 2.83 (4H, s).

### NHS Activated C20 Acid (Intermediate 5)—20-((2,5-dioxopyrrolidin-1-yl)oxy)-20-oxoicosanoic acid

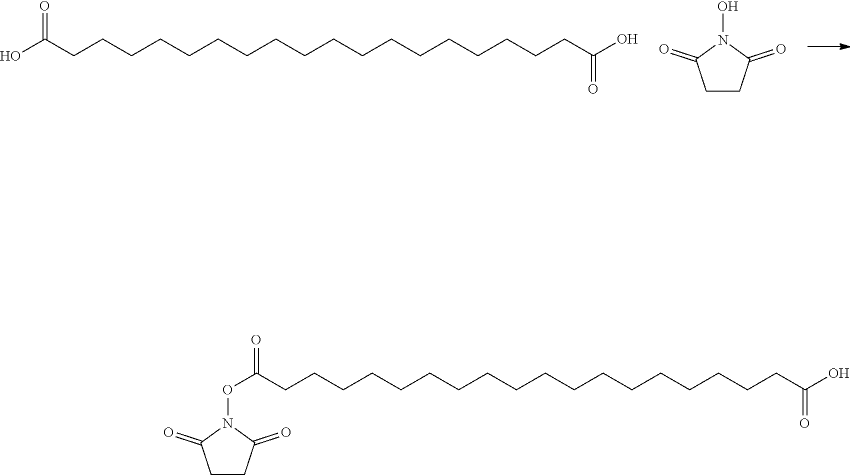

The compound (20-((2,5-dioxopyrrolidin-1-yl)oxy)-20-oxoicosanoic acid) was made according to Intermediate 1 starting from icosanedioic acid (0.3 g, 0.88 mmol). Yield: 229 mg, 59%.

MS (ESI) m/z 438.2 [M-H]⁻

¹H NMR (500 MHz, CDCl₃) 1.2-1.45 (29H, m), 1.63 (2H, qd), 1.74 (2H, p), 2.35 (2H, td), 2.60 (2H, t), 2.83 (4H, d).

### NHS Activated C21 Acid (Intermediate 6)—21-((2,5-dioxopyrrolidin-1-yl)oxy)-21-oxohenicosanoic acid

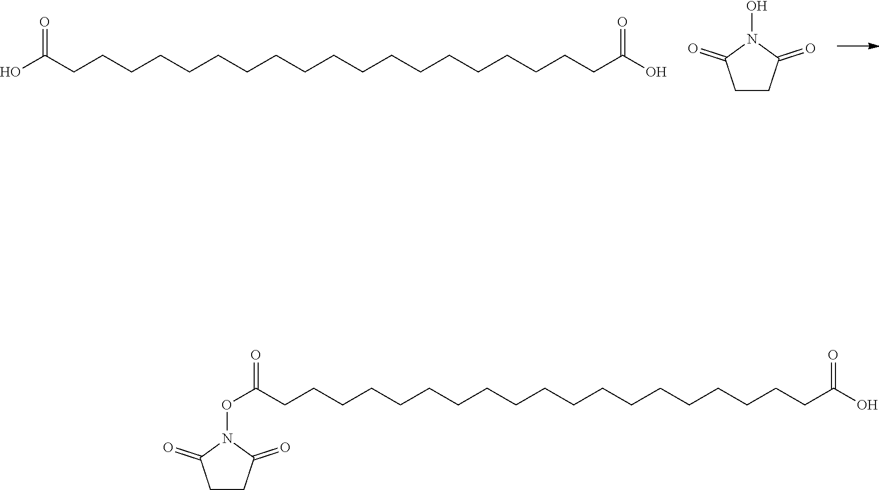 [SMILES: O=C(O)CCCCCCCCCCCCCCCCCCCC(=O)O ; O=C1CCC(=O)N1O ; O=C(O)CCCCCCCCCCCCCCCCCCCC(=O)ON1C(=O)CCC1=O]

The compound was made according to Intermediate 1 starting from henicosanedioic acid (0.3 g, 1.05 mmol). After crystallization, the residue was purified with flash chromatography on silica using ethyl acetate/Heptane 1/1 as eluent. The pure fraction was evaporated to give of the desired product (21-((2,5-dioxopyrrolidin-1-yl)oxy)-21-oxohenicosanoic acid) Yield: 21 mg, (6%).

MS (ESI) m/z 452.0 [M-H]⁻

1H NMR (500 MHz, CDCl3) 1.27 (31H, d), 1.58-1.69 (2H, m), 1.69-1.81 (2H, m), 2.35 (2H, t), 2.60 (2H, t), 2.77-2.91 (4H, m).

### NHS Activated C23 Acid (Intermediate 7)—23-((2,5-dioxopyrrolidin-1-yl)oxy)-23-oxotricosanoic acid

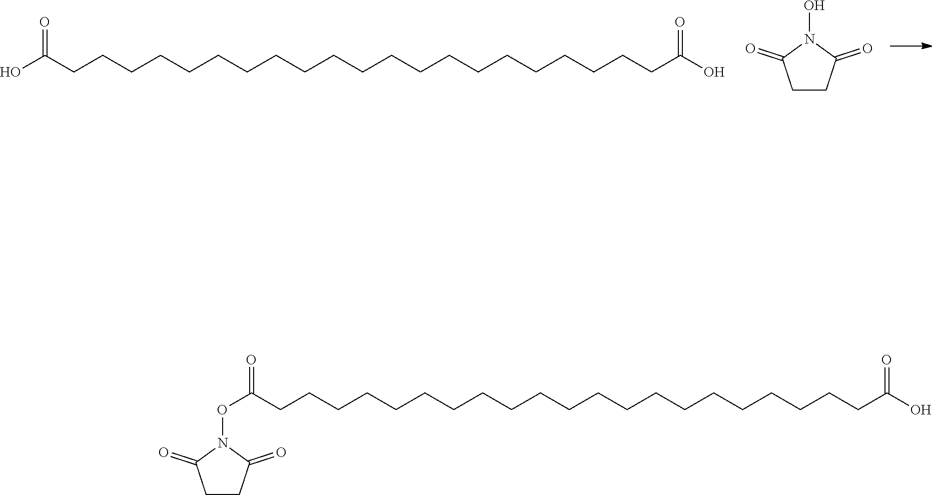 [SMILES: O=C(O)CCCCCCCCCCCCCCCCCCCCCC(=O)O ; O=C1CCC(=O)N1O ; O=C(O)CCCCCCCCCCCCCCCCCCCCCC(=O)ON1C(=O)CCC1=O]

The compound (23-((2,5-dioxopyrrolidin-1-yl)oxy)-23-oxotricosanoic acid) was made according to Intermediate 1 starting from tricosanedioic acid (66 mg, 0.17 mmol). The solids were isolated by centrifugation. Yield: 60 mg, (73%).

MS (ESI) m/z 480.1 [M-H]⁻

¹H NMR (500 MHz, CDCl₃) 1.26 (35H, d), 1.64 (2H, td), 1.74 (2H, p), 2.3-2.38 (2H, m), 2.60 (2H, t), 2.77-2.9 (4H, m).

### NHS Activated C24 Acid (Intermediate 8)—24-((2,5-dioxopyrrolidin-1-yl)oxy)-24-oxotetracosanoic acid

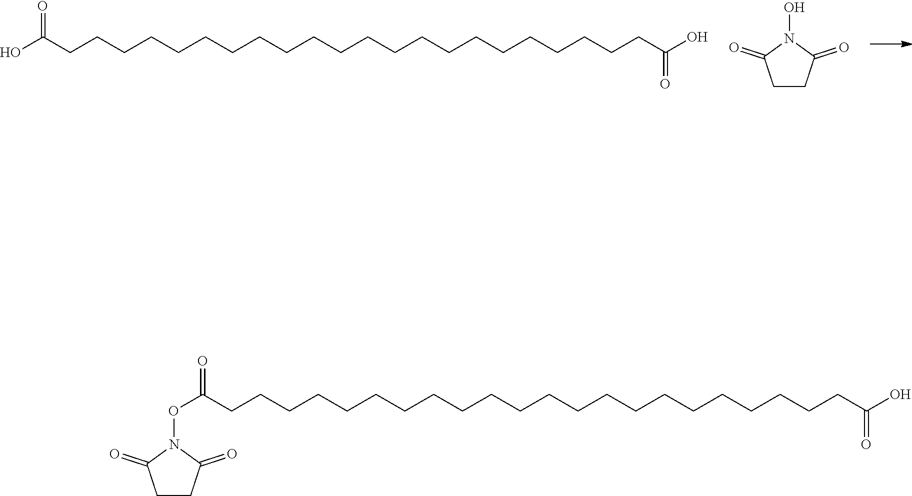

The compound was made according to Intermediate 1 starting from tetracosanedioic acid (0.3 g, 0.75 mmol). After crystallization, the residue was purified with flash chromatography on silica using ethyl acetate/Heptane 1/1 as eluent. The pure fraction was evaporated to give of the desired product (24-((2,5-dioxopyrrolidin-1-yl)oxy)-24-oxotetracosanoic acid) Yield: 44 mg, 12%.

MS (ESI) m/z 494.1 [M-H]⁻

¹H NMR (500 MHz, CDCl₃) 1.25 (37H, s), 1.6-1.67 (2H, m), 1.69-1.79 (2H, m), 2.35 (2H, t), 2.60 (2H, t), 2.84 (4H, d).

### NHS Activated C19 Acid (Intermediate 9)—1-(tert-butyl) 19-(2,5-dioxopyrrolidin-1-yl) nonadecanedioate

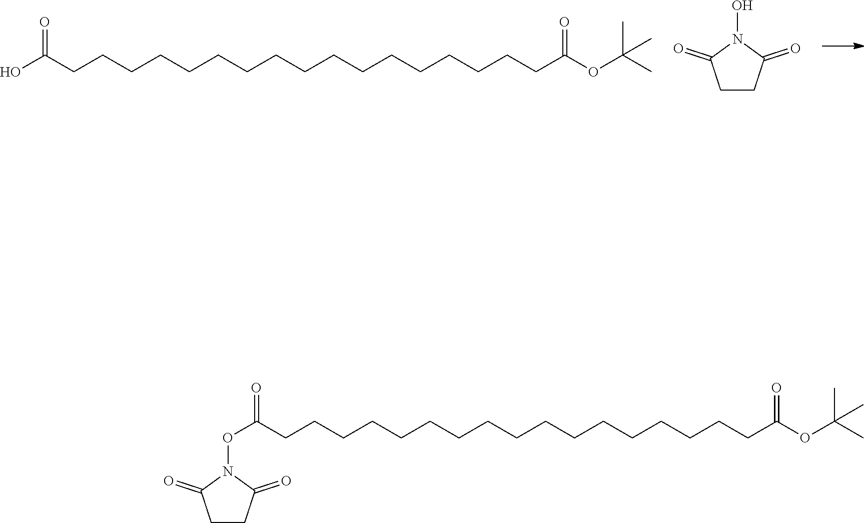

19-(tert-butoxy)-19-oxononadecanoic acid (0.4 g, 1.04 mmol), 1-hydroxypyrrolidine-2,5-dione (0.120 g, 1.04 mmol) and N,N-dimethylpyridin-4-amine (0.635 mg, 5.20 μmol) was added to THF (7 mL) and was stirred at r.t. for 10 min. dicyclohexylmethanediimine (0.215 g, 1.04 mmol) solved in THF (3 mL) was added dropwise during 30 min. and the mixture was then stirred for 24 h. at r.t. After filtration the mixture was evaporated. MeOH (5 ml) was added to the residue, heated to 60° C. and was then stirred for 1 h. at r.t. The product (1-(tert-butyl) 19-(2,5-dioxopyrrolidin-1-yl) nonadecanedioate) was crystalized and was separated by filtration, washed with a small amount of MeOH and dried under reduced pressure. Yield: 361 mg, 72%

¹H NMR (500 MHz, CDCl₃) 1.27 (24H, d), 1.40 (2H, s), 1.44 (9H, s), 1.51-1.63 (2H, m), 1.69-1.8 (2H, m), 2.19 (2H, t), 2.60 (2H, t), 2.83 (4H, d).

### Intermediate 10—19-((2,5-dioxopyrrolidin-1-yl)oxy)-19-oxononadecanoic acid

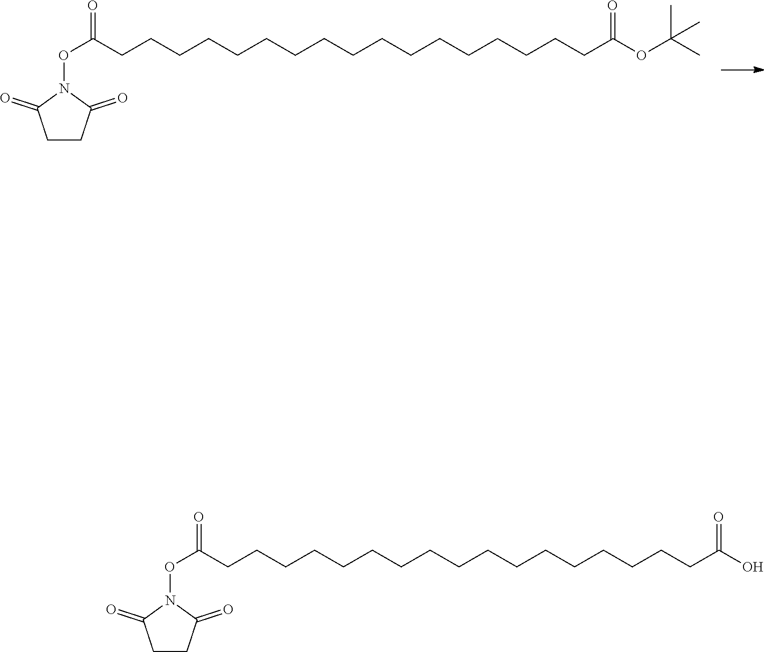 [SMILES: CC(C)(C)OC(=O)CCCCCCCCCCCCCCCCCC(=O)ON1C(=O)CCC1=O ; O=C(O)CCCCCCCCCCCCCCCCCC(=O)ON1C(=O)CCC1=O]

Intermediate 9—1-(tert-butyl) 19-(2,5-dioxopyrrolidin-1-yl) nonadecanedioate (150 mg, 0.31 mmol) was added to 2,2,2-trifluoroacetic acid (3 mL, 0.31 mmol) and the reaction mixture was stirred 4 h. at r.t. The TFA was evaporated and co-evaporated 3 times with toluene and one time with DCM. The residue was solved in MeOH (4 ml) at 60° C. and the solution was stirred at r.t for 15 min. The product was precipitated, filtered and dried under vacuum to give 76 mg (57%) of the desired compound (19-((2,5-dioxopyrrolidin-1-yl)oxy)-19-oxononadecanoic acid).

MS (ESI) m/z 424.0 [M-H]⁻

¹H NMR (500 MHz, CDCl₃) 1.26 (27H, d), 1.63 (2H, q), 1.74 (2H, p), 2.35 (2H, t), 2.60 (2H, t), 2.76-2.9 (4H, m).

### NHS Activated C18 (9Z) Acid (Intermediate 11)—(Z)-18-((2,5-dioxopyrrolidin-1-yl)oxy)-18-oxooctadec-9-enoic acid

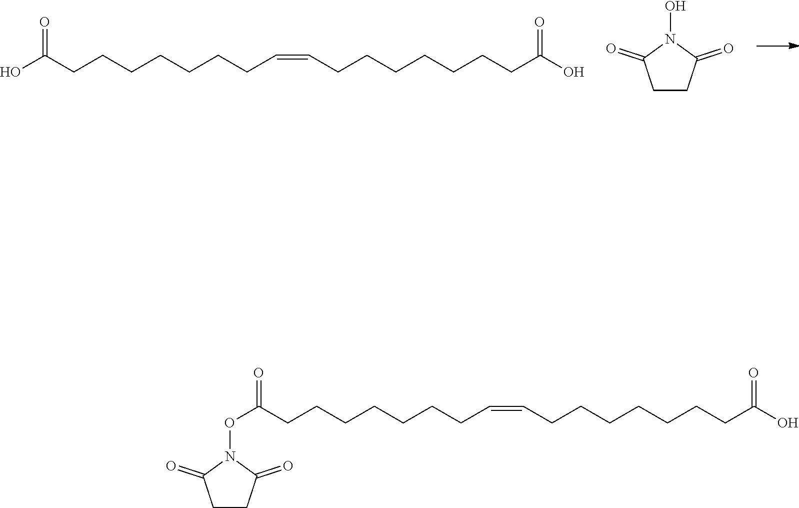 [SMILES: O=C(O)CCCCCCC/C=C\CCCCCCCC(=O)O ; O=C1CCC(=O)N1O ; O=C(O)CCCCCCC/C=C\CCCCCCCC(=O)ON1C(=O)CCC1=O]

(Z)-octadec-9-enedioic acid (88 mg, 0.28 mmol), 1-hydroxypyrrolidine-2,5-dione (32.4 mg, 0.28 mmol) and N,N-dimethylpyridin-4-amine (0.344 mg, 2.82 μmol) was added to THF (2 mL) and was stirred at r.t. for 10 min. The solution was cooled on an icebath to 0 gr and dicyclohexylmethanediimine (58.1 mg, 0.28 mmol) solved in THF (1 mL) was added dropwise during 15 min. and the mixture was then stirred for 24 h. at r.t. After filtration the mixture was evaporated. MeOH (0.5 ml) was added to the residue. The solids were removed by filtration (impurities). and the filtrate was evaporated to give the desired product (Z)-18-((2,5-dioxopyrrolidin-1-yl)oxy)-18-oxooctadec-9-enoic acid). Yield: 90 mg (78%)

MS (ESI) m/z 408.2 [M-H]⁻

¹H NMR (500 MHz, CDCl₃) 1.22-1.45 (16H, m), 1.62 (3H, qd), 1.74 (2H, p), 2.00 (4H, q), 2.3-2.39 (2H, m), 2.59 (2H, t), 2.83 (4H, d), 5.3-5.42 (2H, m).

### Azid C22 Acid (Intermediate 12)—methyl 22-azidodocosanoate

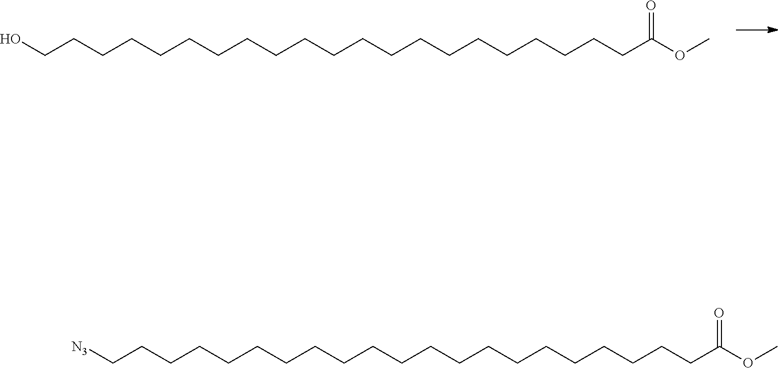 [SMILES: COC(=O)CCCCCCCCCCCCCCCCCCCCCO ; COC(=O)CCCCCCCCCCCCCCCCCCCCCN=[N+]=[N-]]

Diphenyl phosphorazidate (46.4 μl, 0.22 mmol) solved in THF (1 mL) was added to methyl 22-hydroxydocosanoate (40 mg, 0.11 mmol), diisopropyl (E)-diazene-1,2-dicarboxylate (42.5 μl, 0.22 mmol) and triphenylphosphane (56.6 mg, 0.22 mmol) solved in THF (2 mL) at r.t. The reaction mixture was stirred for 3 h. The THF was evaporated and the residue was purified with flash chromatography on silica using heptane/ethylacetate 10/1 as eluent. The product was purified a second time with flash. heptane/ethyl acetate 10/1. The pure fraction was evaporated to give 31 mg, 91% of the desired product (methyl 22-azidodocosanoate).

¹H NMR (500 MHz, CDCl₃) 1.25 (34H, s), 1.54-1.66 (4H, m), 2.29 (2H, t), 3.25 (2H, t), 3.66 (3H, s).

### Intermediate 13—22-azidodocosanoic acid

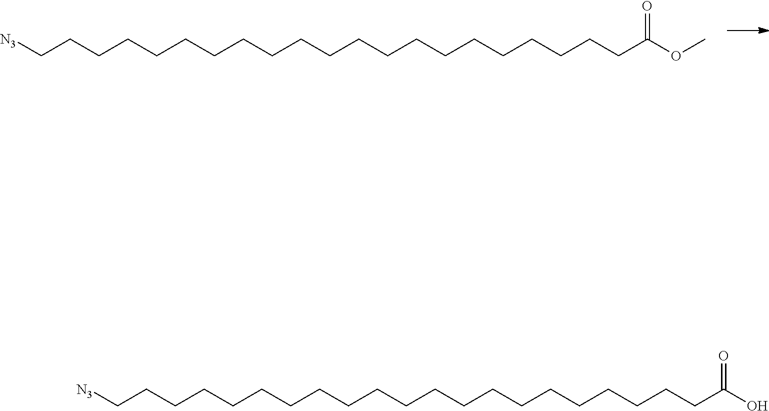 [SMILES: COC(=O)CCCCCCCCCCCCCCCCCCCCCN=[N+]=[N-] ; [N-]=[N+]=NCCCCCCCCCCCCCCCCCCCCCC(=O)O]

Intermediate 12—Methyl 22-azidodocosanoate (35 mg, 0.09 mmol) was added to a solution of lithium hydroxide (10.59 mg, 0.44 mmol) in MeOH (0.5 mL) and water (0.1 mL). The reaction mixture was stirred over night at r.t. Ethyl acetate (1 ml), water (1 ml) and hydrogen chloride (36.9 μl, 0.44 mmol) were added. The organic phase was separated, dried with Na2SO4, filtered and evaporated to give the desired compound (22-azidodocosanoic acid). Yield: 28 mg, 83%

MS (ESI) m/z 380.3 [M-H]⁻

¹H NMR (500 MHz, CDCl₃) 1.25 (35H, s), 1.55-1.67 (4H, m), 2.34 (2H, t), 3.25 (2H, t).

### Intermediate 14—[(1R,8S)-9-bicyclo[6.1.0]non-4-ynyl]methyl activated MALAT1-LNA

MALAT1-LNA-hexylamine was dissolved in water (2400 μl) and triethylamine (28.2 μl, 0.20 mmol) was added. ((1R,8S,9s)-bicyclo[6.1.0]non-4-yn-9-yl)methyl (2,5-dioxopyrrolidin-1-yl) carbonate (9.89 mg, 0.03 mmol) predissolved in acetonitrile (650 μl) was added and the reaction mixture was stirred for 5 min. LCMS showed full conversion. sodium acetate 3M, pH 5.2 (300 μl) was added and the oligo was precipitated by addition of ethanol (24 ml), vortexed briefly and left standing at −20 C overnight. It was centrifuged at 0 C for 10 mins at 3800 rpm, and the clear supernatant was removed and the pellets was dried on vacum. Yield: 101 mg.

MS (ESI⁻) m/z 1424.8 (z=4)

### Intermediate 15—tert-butyl (1-azido-15-{2-[(tert-butoxycarbonyl)amino]ethyl}-11-oxo-3,6,9-trioxa-12,15-diazaheptadecan-17-yl)carbamate

A solution of 2-(2-(2-(2-azidoethoxy)ethoxy)ethoxy)acetic acid (94 mg, 0.40 mmol) in dichloromethane (1 mL) and 0.2 mL of dimethylformamide (0.2 mL) was cooled in ice and N-ethyl-N-isopropylpropan-2-amine (0.140 mL, 0.80 mmol) and 2-(3H-[1,2,3]triazolo[4,5-b]pyridin-3-yl)-1,1,3,3-tetramethylisouronium hexafluorophosphate(V) (153 mg, 0.40 mmol) was added. The mixture was stirred for 5 min and then a solution of di-tert-butyl (((2-aminoethyl)azanediyl)bis(ethane-2,1-diyl))dicarbamate (CAS 161038-11-5) (139 mg, 0.40 mmol) in dichloromethane and dimethylformamide (0.2 mL) was added. The yellow reaction mixture was stirred for 1 h at rt. The reaction mixture was diluted with ethyl acetate and washed with water and brine. The organic phase was dried over magnesium sulphate, filtered and evaporated to dryness. The compound was purified by preparative HPLC on a XBridge C18 column (10 μm 250×50 ID mm) using a gradient of 20-75% acetonitrile in ammonia(0.2%) buffer over 20 minutes with a flow of 100 mL/min. The product was detected by LS-MS analyses. Product fractions were concentrated to give the desired compound (tert-butyl (1-azido-15-{2-[(tert-butoxycarbonyl)amino]ethyl}-11-oxo-3,6,9-trioxa-12,15-diazaheptadecan-17-yl)carbamate).

Yield 131 mg (58.1%)

MS (ESI⁺) m/z 562.5 (z=1)

1H NMR (500 MHz, CDCl3) 1.46 (18H, s), 3.30 (4H, s), 3.4-3.47 (3H, m), 3.48-3.63 (4H, m), 3.72 (13H, dq), 4.05 (2H, s), 5.84 (2H, s), 7.87 (1H, s).

### Intermediate 16—1-azido-15-(2-(21-carboxyhenicosanamido)ethyl)-11,19-dioxo-3,6,9-trioxa-12,15,18-triazatetracontan-40-oic acid

To a solution of Intermediate 15 (60 mg, 110 μmol) and triisopropylsilane (5 μl, 110 μmol) in dichloromethane (500 μl) was added 2,2,2-trifluoroacetic acid (500 μl) at rt. The mixture was stirred at rt for 5 h and evaporated to dryness (MS (ESI⁺) m/z 362.4 (z=1). To the crude TFA-salt (15 mg, 30 μmol) was added acetonitrile (100 μl), water (100 μl), triethylamine (28.3 μl, 0.21 mmol) and N,N-dimethylpyridin-4-amine (0.156 mg, 1.28 μmol) to give a clear solution. Intermediate 1 (40 mg, 90 μmol) dissolved in tetrahydrofuran (300 μl) was added and the mixture was stirred at at 45° C. for 4 h. The reaction was evaporated, diluted with DMSO and purified by preparative HPLC on a XBridge C18 column (10 μm 250×50 ID mm) using a gradient of 20-75% acetonitrile in ammonia(0.2%) buffer over 20 minutes with a flow of 100 mL/min. The product was detected by LS-MS analyses. Product fractions were concentrated to give the desired compound (1-azido-15-(2-(21-carboxyhenicosanamido)ethyl)-11,19-dioxo-3,6,9-trioxa-12,15,18-triazatetracontan-40-oic acid). Yield 4.4 mg (16.2%)

MS (ESI⁺) m/z 1066.6 (z=1)

### Intermediate 17—Maleimide activated MALAT1-LNA

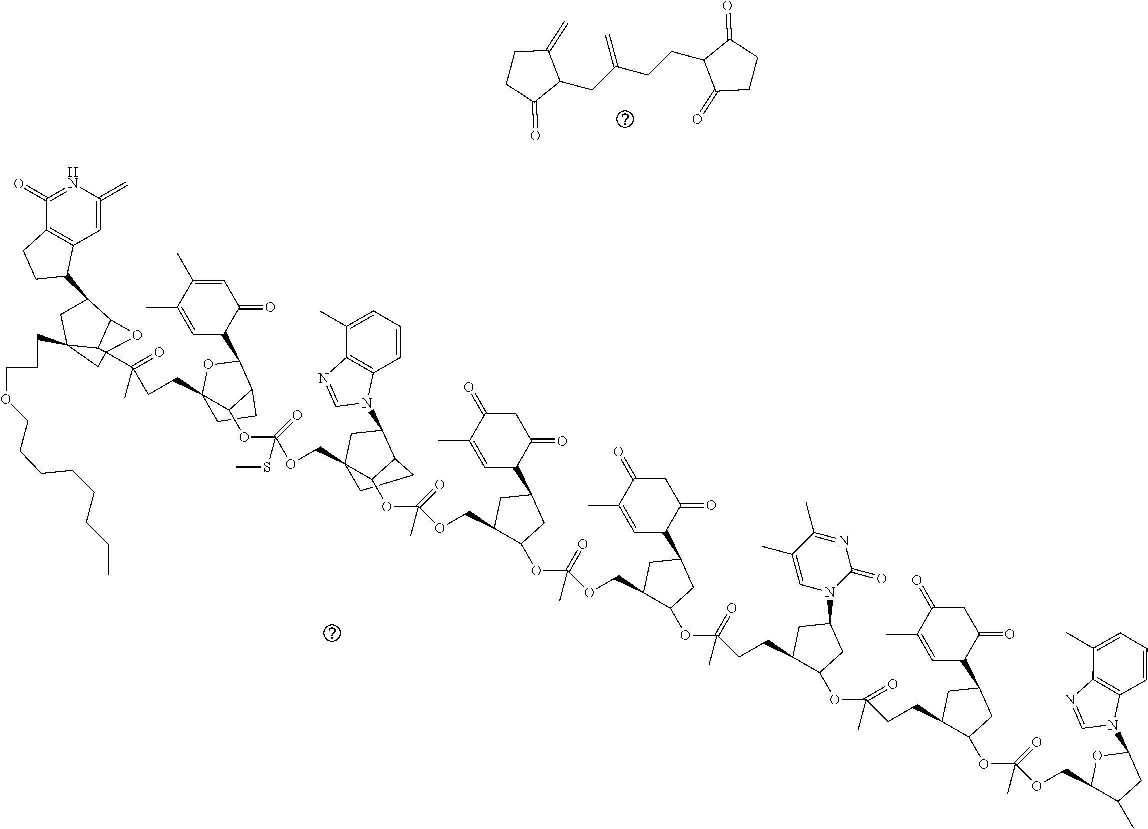

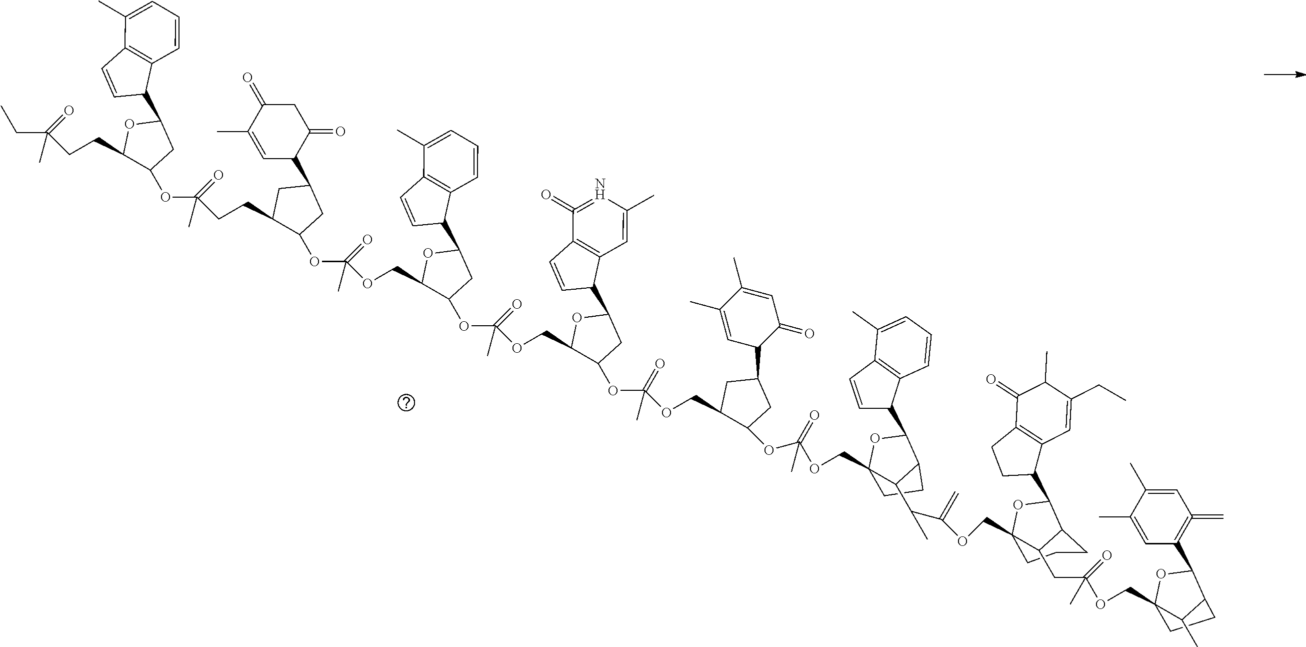

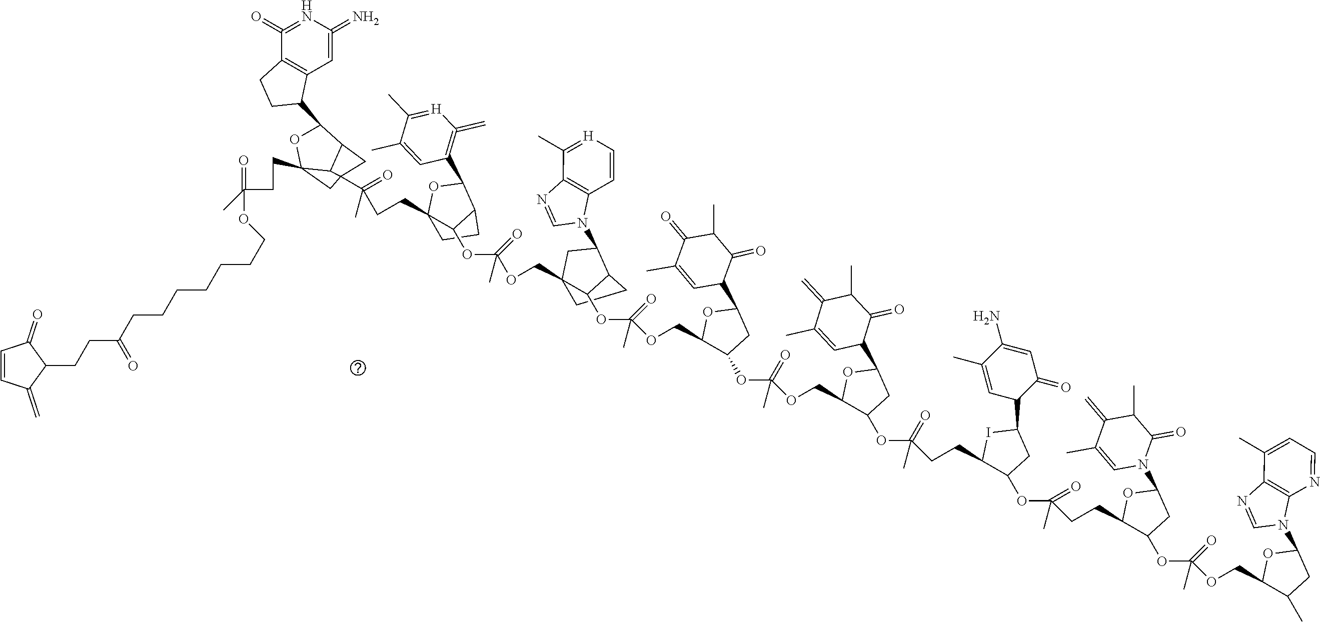

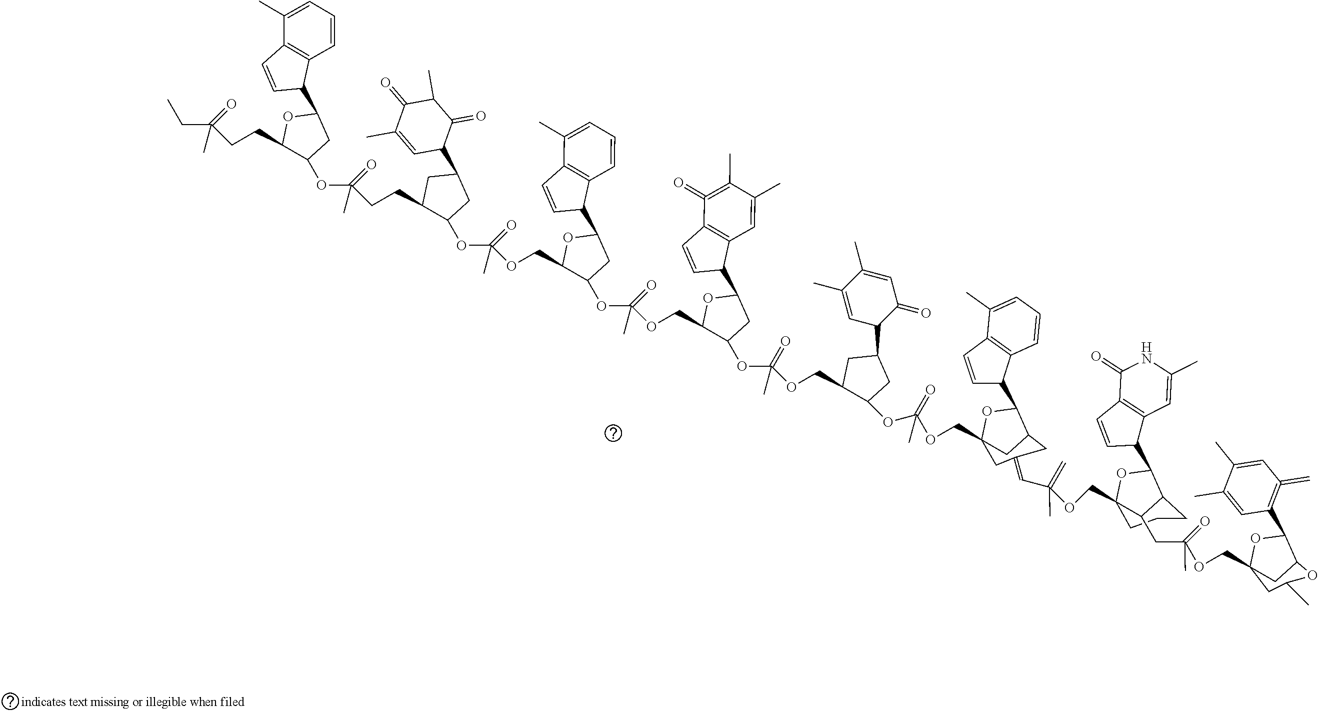

MALAT1-LNA-hexylamine was dissolved in Phosphate buffer (0.1M pH 7.34, 660 μl) and 2,5-dioxopyrrolidin-1-yl 3-(2,5-dioxo-2,5-dihydro-1H-pyrrol-1-yl)propanoate (9.76 mg, 0.04 mmol) dissolved in acetonitrile (330 μL) was added. The clear solution was stirred for 1.5 h at rt. Sodium acetate (3M, pH 5.2, 100 μl) was added and then the oligo was precipitated by addition of ethanol (4 mL), vortexed briefly and left standing at −20 C for 30 min. The mixture was centrifuged at 0° C. for 10 mins at 3500 rpm and the clear supernatant was removed. The oligo was washed once more by dissolving the pellet in 1 mL of water and Sodium acetate 3M, pH 5.2 (100 μl) (shaking necessary for full dissolution) followed by addition of 4 mL of ethanol, cooled for 30 mins at −20, then centrifuged at 0° C. for 10 mins at 3500 rpm. The supernatant was discarded, and the pellet was dried under a nitrogen flow. Yield 27 mg (97%)

MS (ESI⁻) m/z 1418.1 (z=4)

### Intermediate 18—di-tert-butyl 22,22′-(((2R,2′R)-disulfanediylbis(3-(tert-butoxy)-3-oxopropane-1,2-diyl))bis(azanediyl))bis(22-oxodocosanoate)

To 22-(tert-butoxy)-22-oxodocosanoic acid (87 mg, 0.20 mmol) in N-methyl-pyrrolidinone (1 ml) was added DIPEA (31.7 μl, 0.18 mmol) and 2-(3H-[1,2,3]triazolo[4,5-b]pyridin-3-yl)-1,1,3,3-tetramethylisouronium hexafluorophosphate(V) (69 mg, 0.18 mmol). di-tert-butyl 3,3′-disulfanediyl(2R,2′R)-bis(2-aminopropanoate) (32 mg, 0.09 mmol) in dimethylformamide (1 ml) was then added. The resulting opaque reaction mixture was stirred for 24 h at rt. The mixture was diluted with diethyl ether and washed with water, sodium bicarbonate (10%), potassium hydrogen sulphate (0.5M), water and brine. The organic phase was evaporated, and the residue was passed through a 2 g silica plug with heptane and increasing diethyl ether. The product was detected by TLC with cerium ammonium molybdate stain. Pure product fractions were evaporated to give the desired product (di-tert-butyl 22,22′-(((2R,2′R)-disulfanediylbis(3-(tert-butoxy)-3-oxopropane-1,2-diyl))bis(azanediyl))bis(22-oxodocosanoate). Yield: 85 mg, (80%).

1H NMR (500 MHz, CDCl3) 1.27 (64H, s), 1.47 (19H, s), 1.50 (17H, s), 1.55-1.62 (4H, m), 1.66 (4H, t), 2.22 (4H, t), 2.27 (4H, td), 3.22 (4H, dd), 4.77 (2H, dt), 6.46 (2H, d).

### Intermediate 19—22,22′-(((1R,1′R)-disulfanediylbis(1-carboxyethane-2,1-diyl))bis(azanediyl))bis(22-oxodocosanoic acid)

To a solution of Intermediate 18 (15 mg, 10 μmol) in dichloromethane (100 μl) was added 2,2,2-trifluoroacetic acid (500 μl) at rt. The mixture was stirred at rt for 15 h and evaporated to dryness and then co-evaporated with toluene to give the desired compound (22,22′-(((1R,1′R)-disulfanediylbis(1-carboxyethane-2,1-diyl))bis(azanediyl))bis(22-oxodocosanoic acid)).

Yield 12 mg (99%)

MS (ESI⁺) m/z 944.1 (z=1)

### Intermediate 21—(1-{[(2R)-1-amino-1-oxo-3-sulfanylpropan-2-yl]amino}-1,10,19-trioxo-3,6,12,15-tetraoxa-9,18-diazatetracontan-40-oic acid)

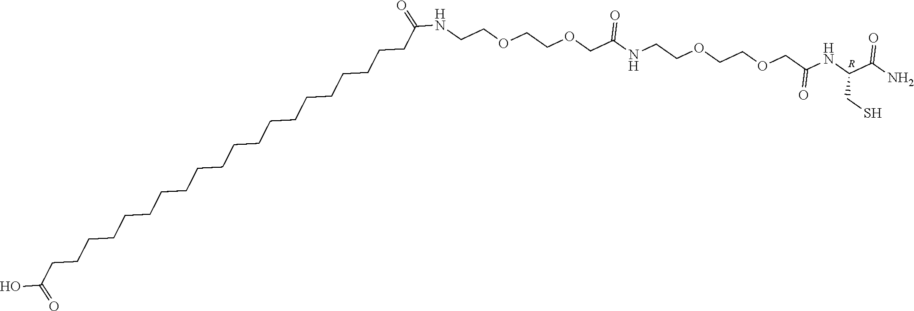

The synthesis was performed by Automated Solid Phase Synthesis in a Biotage Alstra peptide synthesizer equipped with a microwave heater. Rink amide Chem Matrix resin (0.4 g, 0.16 mmol, loading 0.4 mmol/g) was weighed into a 10 mL reaction vial and swelled twice in DMF for 10 min at 55° C. under agitation. Solutions of N-(((9H-fluoren-9-yl)methoxy)carbonyl)-S-trityl-L-cysteine (0.387 g, 0.2M, 3.3 mL, 0.66 mmol), Oxyma (0.5M, 4 mL, 1.3 mL, 0.66 mmol) and diisopropylcarbodiimide (2M, 0.265 mL, 0.53 mmol) in NMP was added and the reaction was agitated at 40° C. for 20 min. The resin was washed with DMF (4×) and treated twice with piperidine (20% in DMF) for 3+10 min. The same coupling procedure was repeated twice for 1-(9H-fluoren-9-yl)-3-oxo-2,7,10-trioxa-4-azadodecan-12-oic acid.

Solutions of 22-(tert-butoxy)-22-oxodocosanoic acid (137 mg, 0.32 mmol) (4 eq, in DMF/NMP 1:1, 4 mL), 1-(bis(dimethylamino)methylene)-1H-[1,2,3]triazolo[4,5-b]pyridine-1-ium 3-oxide hexafluorophosphate(V) (HATU) (122 mg, 0.32 mmol) and N-ethyl-N-isopropylpropan-2-amine (62.9 μl, 0.36 mmol) were then added to the resin and the reaction was agitated at rt for 1 h45 min. The resin was finally washed with DMF (4×), methanol and DCM. A mixture of TFA/H2O/DODT//TIPS (94/2.5/2.5/1) (3 mL) was added to the resin and the reaction mixture was agitated at room temperature for 2 h. The resin was filtered off and washed with TFA (−2 mL) which was combined with the filtrate. The product was precipitated in cold Et2O. The precipitated product was centrifugated and the supernatant discarded. The solid material was washed three times by addition of cold Et20 and centrifugation. The resulting solid material was suspended in MeCH/H2O/TFA (50/50/0.1) and freeze dried.

The compound was purified by preparative HPLC on a Kromasil C8 column (10 μm 250×50 ID mm) using a gradient of 35-80% acetonitrile in H2O/ACN/FA 95/5/0.2 buffer over 20 minutes with a flow of 100 mL/min. The compound was detected by UV at 220 nm. The product fractions were freeze dried to give the desired compound.

Yield 27 mg (44%)

1H NMR (500 MHz, CDCl₃) 1.27 (32H, s), 1.61-1.67 (4H, m), 2.23 (2H, t), 2.34 (2H, t), 2.65 (3H, s), 2.85 (1H, ddd), 3.09 (1H, ddd), 3.45-3.54 (3H, m), 3.59 (2H, t), 3.61-3.73 (10H, m), 3.78 (1H, qd), 4.05 (2H, s), 4.78 (1H, ddd), 6.52 (2H, d), 6.75 (1H, s), 7.32 (1H, t), 7.78 (1H, d). Carboxylic acid proton not integrated.

MS (ESI⁺) m/z 763.9 (z=1)

### Intermediate 22—(S)-1-amino-22-carboxy-2,11,20,28-tetraoxo-6,9,15,18-tetraoxa-3,12,21,27-tetraazanonatetracontan-49-oic acid

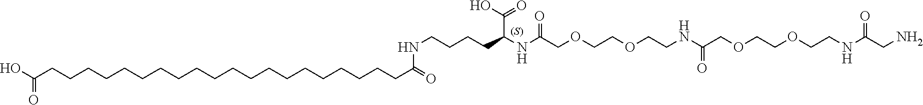

The resin bound precursor (S)-23-(4-((1-(4,4-dimethyl-2,6-dioxocyclohexylidene)-3-methylbutyl)amino)butyl)-1-(9H-fluoren-9-yl)-3,12,21-trioxo-2,7,10,16,19-pentaoxa-4,13-diazatetracosan-24-oic acid was synthesized according to the methods described for coupling and deprotection steps for the synthesis of intermediate 21 staring from Wang resin bound (S)-2-((((9H-Fluoren-9-yl)methoxy)carbonyl)amino)-6-((1-(4,4-dimethyl-2,6-dioxocyclohexylidene)-3-methylbutyl)amino)hexanoic acid (Fmoc-Lys-IvDde-Wang resin, loading 0.6 mmol/g, 0.3 g,), 1-(9H-fluoren-9-yl)-3-oxo-2,7,10-trioxa-4-azadodecan-12-oic acid and (tert-butoxycarbonyl)glycine. The IvDde-group was then removed by treating the resin with 5% Hydrazine/DMF six times at rt for 2+5+5+5+5+5 min. The same procedures used in the synthesis of intermediate 21 was then performed for the coupling of 22-(tert-butoxy)-22-oxodocosanoic acid (137 mg, 0.32 mmol) and for the cleavage and purification of the final product.

MS (ESI⁺) m/z 846.7 (z=1)

1H NMR (500 MHz, DMSO) 1.23 (34H, s), 1.33 (2H, p), 1.46 (4H, dt), 1.56 (1H, d), 1.68 (1H, d), 2.00 (2H, t), 2.17 (2H, t), 2.95 (2H, q), 3.28 (3H, p), 3.32 (1H, d), 3.42 (2H, s), 3.47 (4H, q), 3.51-3.63 (8H, m), 3.84 (2H, s), 3.88-3.96 (3H, m), 7.58 (1H, d), 7.71 (1H, d), 7.87 (1H, t), 8.45 (1H, s). NH2 and 2×CO2H not integrated

### Example 1—Synthesis of C₂₀ Saturated Acid Conjugate of Malat-1 LNA Antisense Oligonucleotide (SEQ ID No: 8)

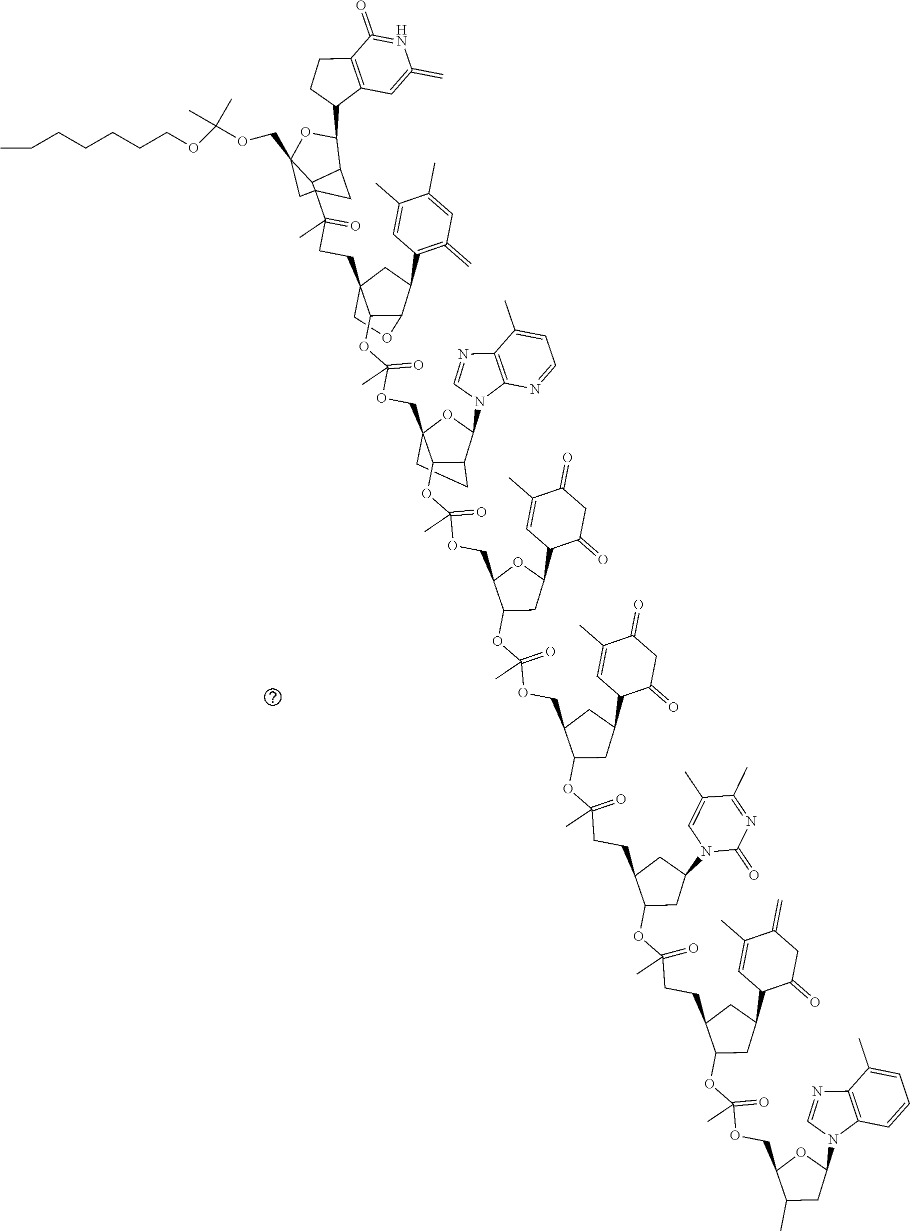

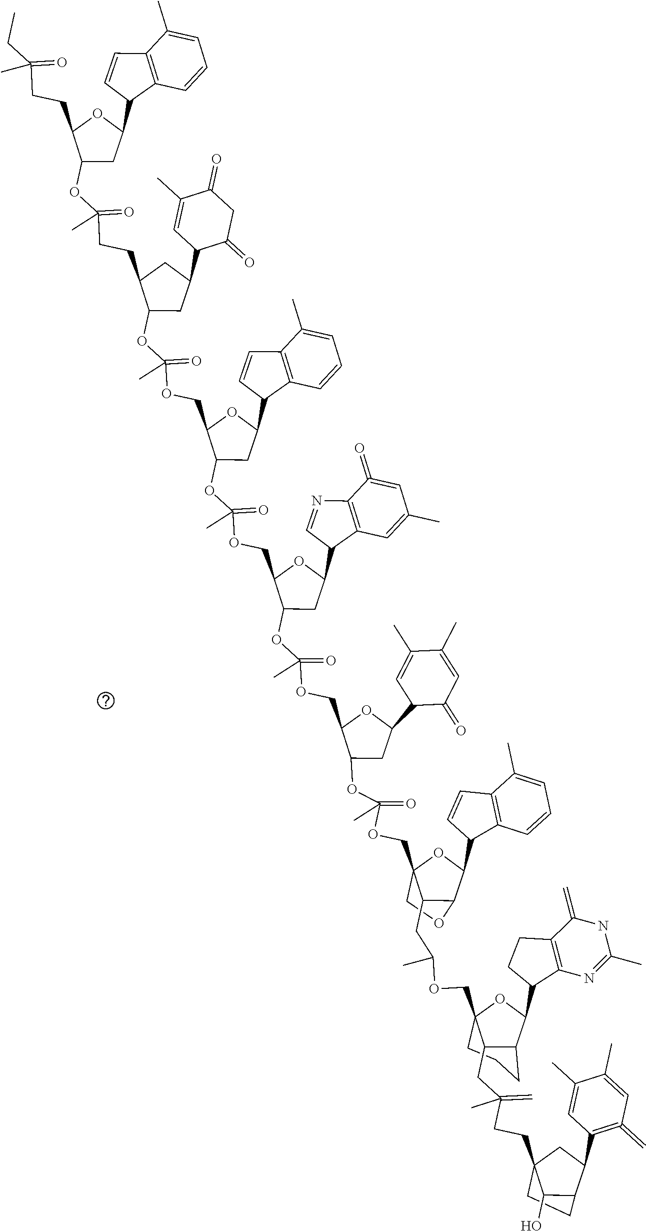

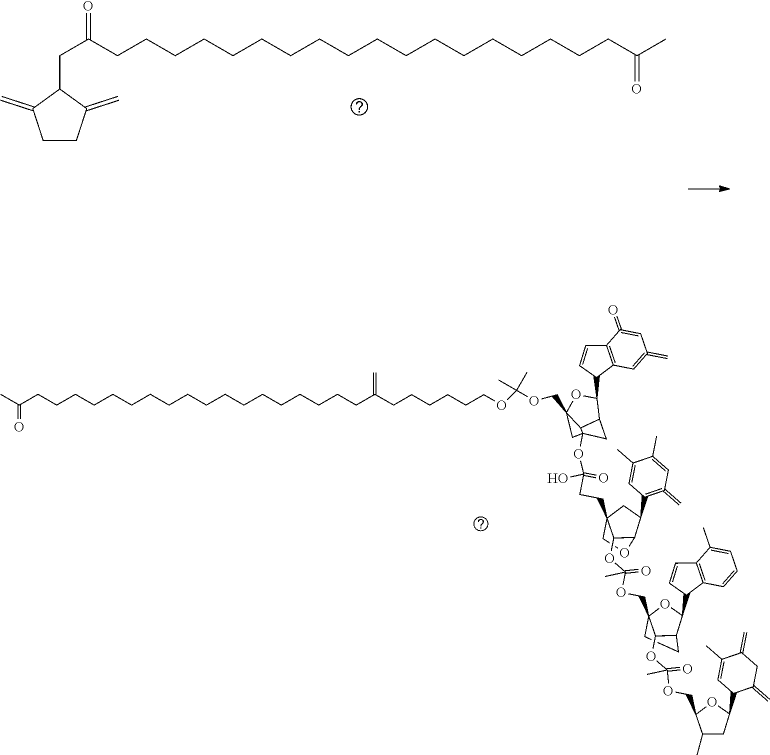

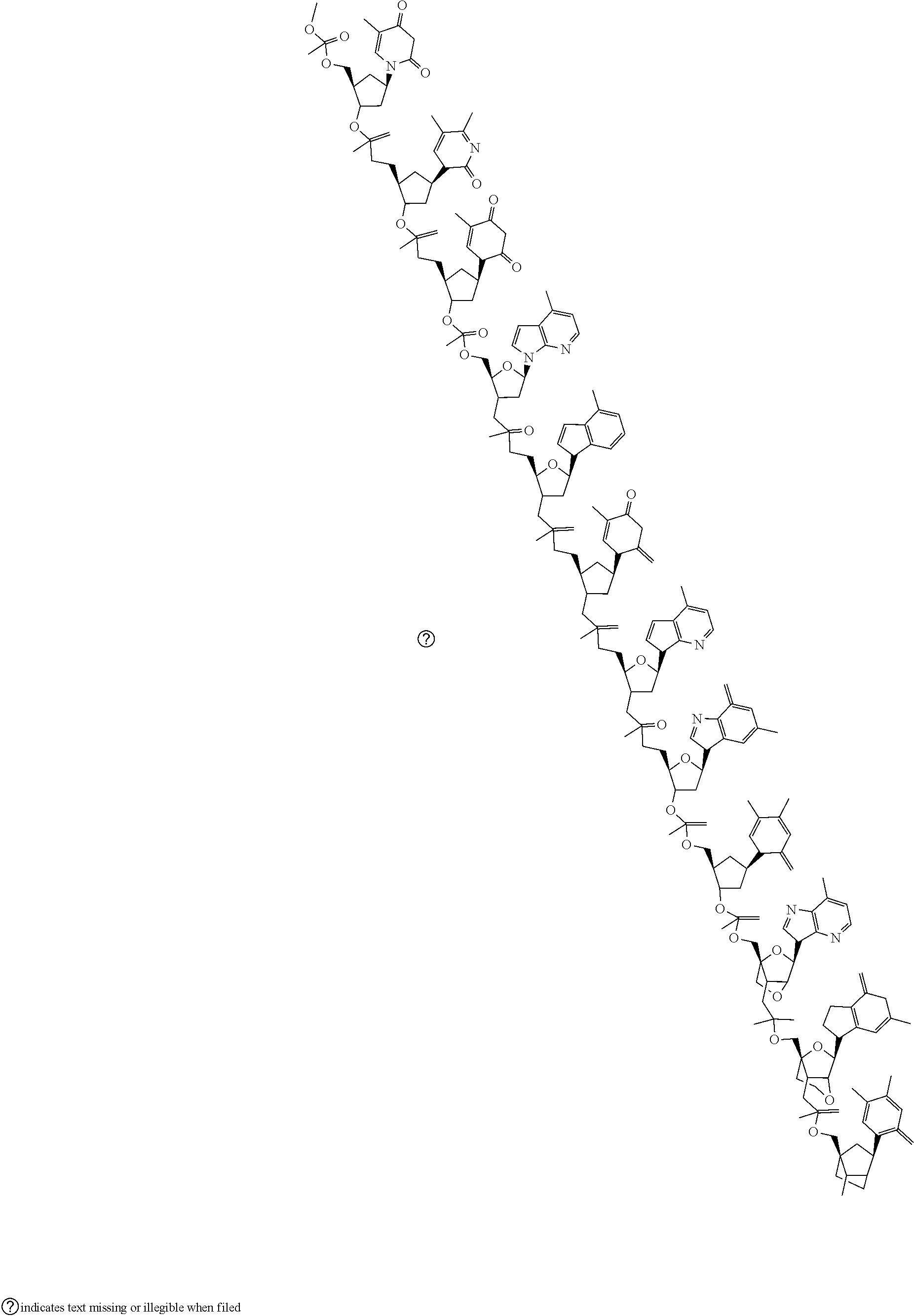

MALAT1-LNA-hexylamine (34 mg, 6.15 μmol) was dissolved in water (800 μl) and triethylamine (9.53 μl, 0.07 mmol) was added. 20-((2,5-dioxopyrrolidin-1-yl)oxy)-20-oxoicosanoic acid (4.06 mg, 9.23 μmol) (Intermediate 5) solved in a mixture of warm (60° C.) acetonitrile (200 μl) and DMSO (120 μl) was added to the solved oligonucleotide and the reaction mixture was stirred at RT for 60 min. Sodium acetate 3M, pH 5.2 (90 μl) was added and then the oligo-conjugate was precipitated by addition of ethanol (8000 μl), The mixture was centrifuged at 0° C. for 10 mins at 3500 rpm and the clear supernatant was removed. The pellet was dried under vacuum and the residue was purified on a XBridge C18, 5 μm 19×150 mm column by using a gradient from 5-90% acetonitrile in NH₄HCO₃ (50 mM, pH8) at R.T. The pure fractions was freeze-dried twice to give the desired compound as ammonium salt. The yield was 15 mg (40%).

MS (ESI⁻) m/z 1461.4 (z=4)

### Example 2—Synthesis of C₁₆ Saturated Acid Conjugate of Malat-1 LNA Antisense Oligonucleotide (SEQ ID NO: 8)

The Example 2 compound was made according to Example 1 (C20-acid-MALAT1-LNA) starting from MALAT1-LNA-hexylamine (36 mg, 6.52 μmol) and 16-((2,5-dioxopyrrolidin-1-yl)oxy)-16-oxohexadecanoic acid (5.00 mg, 13.0 μmol (Intermediate 2). The yield was 20 mg (50%).

MS (ESI⁻) m/z 1447.5 (z=4)

### Example 3—Synthesis of C₁₇ Saturated Acid Conjugate of Malat-1 LNA Antisense Oligonucleotide (SEQ ID NO: 8)

The Example 3 compound was made according to Example 1 (C20-acid-MALAT1-LNA) starting from MALAT1-LNA-hexylamine (34 mg, 6.15 μmol) and 17-((2,5-dioxopyrrolidin-1-yl)oxy)-17-oxoheptadecanoic acid (4.89 mg, 12.3 μmol) (Intermediate 3) Reaction time 10 min. The yield was 18 mg (48%).

MS (ESI⁻) m/z 1450.9 (z=4)

### Example 4—Synthesis of C₁₈ Saturated Acid Conjugate of Malat-1 LNA Antisense Oligonucleotide (SEQ ID NO: 8)

The Example 4 compound was made according to Example 1 (C20-acid-MALAT1-LNA) starting from MALAT1-LNA-hexylamine (38 mg, 6.88 μmol) and triethylamine (9.53 μl, 0.07 mmol) was added. 18-((2,5-dioxopyrrolidin-1-yl)oxy)-18-oxooctadecanoic acid (5.66 mg, 13.8 μmol) (Intermediate 4). Reaction time: 90 min. Purification on a XBridge C18, 5 μm 19×150 mm column by using a gradient from 15-90% acetonitrile in NH₄HCO₃ (50 mM, pH8) at R.T. Yield: 24 mg (57%).

MS (ESI⁻) m/z 1454.4 (z=4)

### Example 5—Synthesis of C₁₉ Saturated Acid Conjugate of Malat-1 LNA Antisense Oligonucleotide (SEQ ID NO: 8)

The compound was made according to Example 1 (C20-acid-MALAT1-LNA) starting from MALAT1-LNA-hexylamine (34 mg, 6.15 μmol) and 19-((2,5-dioxopyrrolidin-1-yl)oxy)-19-oxononadecanoic acid (5.24 mg, 12.3 mmol) (Intermediate 10). Reaction time: 30 min. The yield was 21 mg (56%).

MS (ESI⁻) m/z 1457.7 (z=4)

### Example 6—Synthesis of C₂₁ Saturated Acid Conjugate of Malat-1 LNA Antisense Oligonucleotide (SEQ ID NO: 8)

The compound was made according to Example 1 (C20-acid-MALAT1-LNA) starting from MALAT1-LNA-hexylamine (34 mg, 6.15 μmol) and 21-((2,5-dioxopyrrolidin-1-yl)oxy)-21-oxohenicosanoic acid (5.58 mg, 12.3 μmol 1) (Intermediate 6). Yield 20 mg (53%).

MS (ESI⁻) m/z 1465.1 (z=4)

### Example 7—Synthesis of C₂₂ Saturated Acid Conjugate of Malat-1 LNA Antisense Oligonucleotide (SEQ ID NO: 8)

MALAT1-LNA-hexylamine (100 mg, 0.02 mmol) and triethylamine (10.03 μl, 0.07 mmol) dissolved in water (2.4 ml) and 22-((2,5-dioxopyrrolidin-1-yl)oxy)-22-oxodocosanoic acid (21.16 mg, 0.05 mmol) (Intermediate 1) dissolved in warm acetonitrile (400 μl) and DMSO (200 μl) was added. Stirred at 40° C. for 24 h. Additional 2.5 eqv of the activated lipid was added and the mixture stirred additional 24 h. at 40° C. Sodium acetate 3M, pH 5.2 (270 μl) was added and then the oligo was precipitated by addition of ethanol (24 ml). The mixture was centrifuged at 0° C. for 10 mins at 3500 rpm and the clear supernatant was removed. The residue was washed with EtOH (5 ml.) and the pellet was dried under vacuum. The residue was purified on a XBridge C18, 5 μm 19×150 mm column by using a gradient from 15-90% acetonitrile in NH₄HCO₃ (50 mM, pH8) at R.T. The pure fractions was freeze-dried twice. Yield: 28 mg (25%).

MS (ESI⁻) m/z 1468.6 (z=4)

### Example 8—Synthesis of C₂₃ Saturated Acid Conjugate of Malat-1 LNA Antisense Oligonucleotide (SEQ ID NO: 8)

The compound was made according to Example 1 (C20-acid-MALAT1-LNA) starting from MALAT1-LNA-hexylamine (39 mg, 7.06 μmol) and 23-((2,5-dioxopyrrolidin-1-yl)oxy)-23-oxotricosanoic acid (8.50 mg, 15.2 μmo) (Intermediate 7). The yield was 17 mg (39%).

MS (ESI−) m/z 1472.5 (z=4)

### Example 9—Synthesis of C₂₄ Saturated Acid Conjugate of Malat-1 LNA Antisense Oligonucleotide (SEQ ID NO: 8)

The compound was made according to Example 1 C20-acid-MALAT1-LNA starting from MALAT1-LNA-hexylamine (41 mg, 7.42 μmol) and 24-((2,5-dioxopyrrolidin-1-yl)oxy)-24-oxotetracosanoic acid (7.36 mg, 0.01 mmol) (Intermediate 8). Reaction time: over night. The yield was 23 mg (50%).

MS (ESI⁻) m/z 1476.0 (z=4)

### Example 10—Synthesis of C₁₈ (9Z) Saturated Acid Conjugate of Malat-1 LNA Antisense Oligonucleotide (SEQ ID NO: 8)

MALAT1-LNA-hexylamine (180 mg, 0.03 mmol) was dissolved in Borate buffer 0.1M pH 9.5 (4.8 ml). (Z)-18-((2,5-dioxopyrrolidin-1-yl)oxy)-18-oxooctadec-9-enoic acid (Intermediate 11) (26.7 mg, 0.07 mmol) dissolved in acetonitrile (1.2 mL) was added and the mixture was stirred at RT for 45 m. Additional 1 eqv of (Intermediate 11) was added and the mixture was stirred for additional 2 h. 1M NaOH solution (1.4 ml) was added during 1 h. Sodium acetate 3M, pH 5.2 (540 μL) was added and then the oligo was precipitated by addition of ethanol (48 ml). The mixture was centrifuged at 0° C. for 10 mins at 3500 rpm, the clear supernatant was removed. The pellet was dried under vacuum. The residue was purified on a XBridge C18, 5 μm 19×150 mm column by using a gradient from 5-90% acetonitrile in NH₄HCO₃ (50 mM, pH8) at R.T. The pure fractions was freeze-dried twice. Yield: 79 mg (40%).

MS (ESI⁻) m/z 1454.1 (z=4)

### Example 11—Synthesis of C₂₂ Click Saturated Acid Conjugate OF Malat-1 LNA Antisense Oligonucleotide (SEQ ID NO: 8)

[(1R,8S)-9-bicyclo[6.1.0]non-4-ynyl]methyl activated MALAT1-(Intermediate 14) (50 mg, 8.77 μmol) was added to a mixture of 22-azidodocosanoic acid (5.02 mg, 0.01 mmol) (Intermediate 13) in DMSO (0.2 mL) and acetonitrile (0.200 mL). water (0.5 mL) was added and the mixture was stirred for 3 h. Sodium acetate 3M, pH 5.2 (120 μL) and ethanol (10 mL) was added. The mixture was centrifuged at 0° C. for 10 mins at 3500 rpm, the clear supernatant was removed. The pellet was dried under vacuum. The residue was purified on a XBridge C18, 5 μm 19×150 mm column by using a gradient from 10-90% acetonitrile in NH₄HCO₃ (50 mM, pH8) at R.T. The pure fractions was freeze-dried twice. Yield: 5.0 mg.

MS (ESI⁻) m/z 1520.1 (z=4)

### Example 12—Synthesis of C₂₂ Saturated Acid Conjugate of Camk2D Antisense Oligonucleotide (SEQ ID NO: 9)

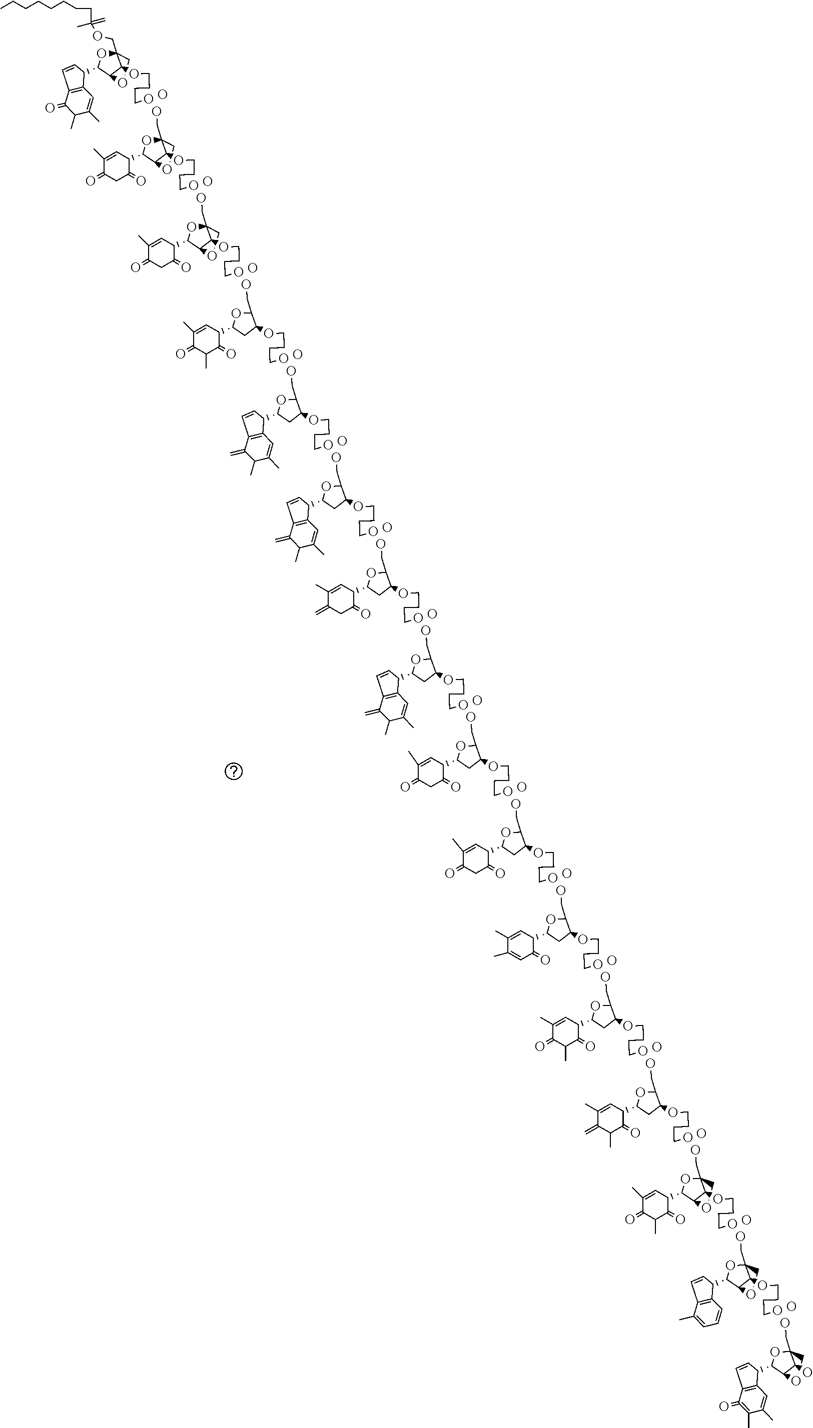

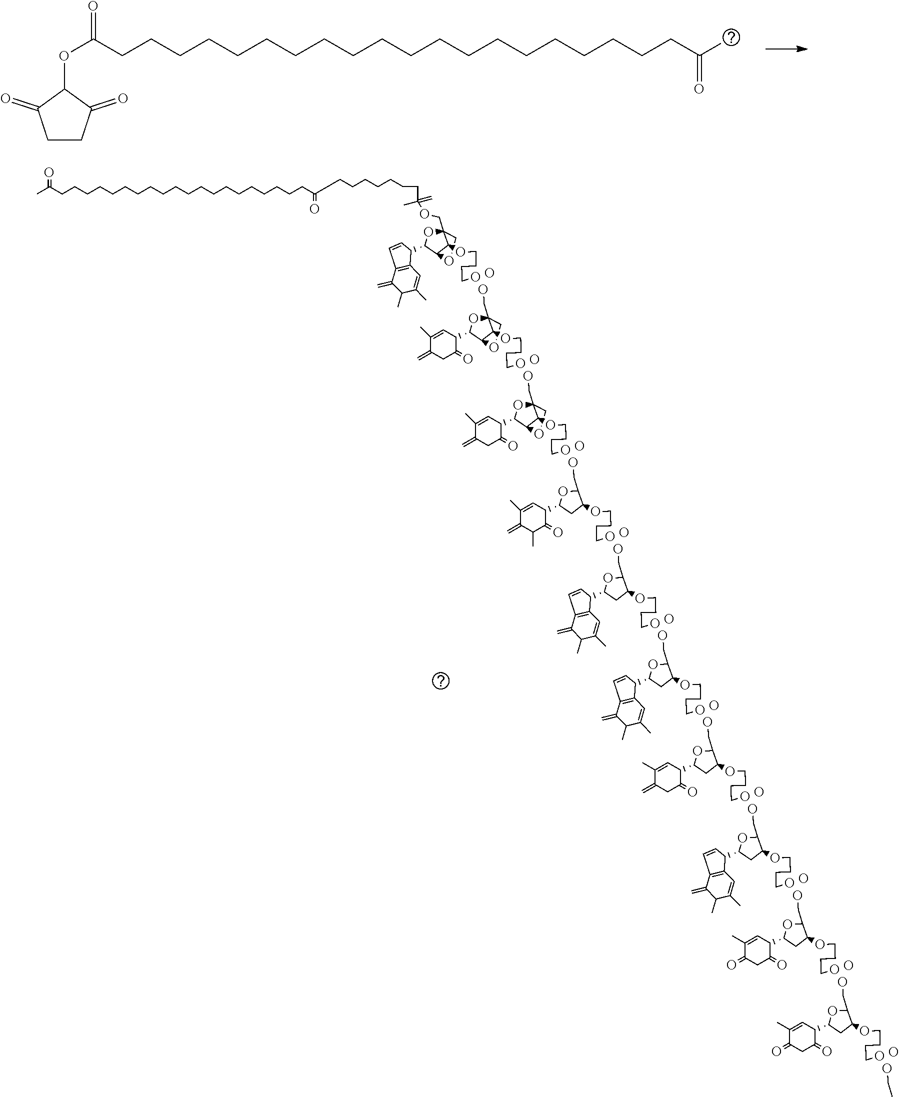

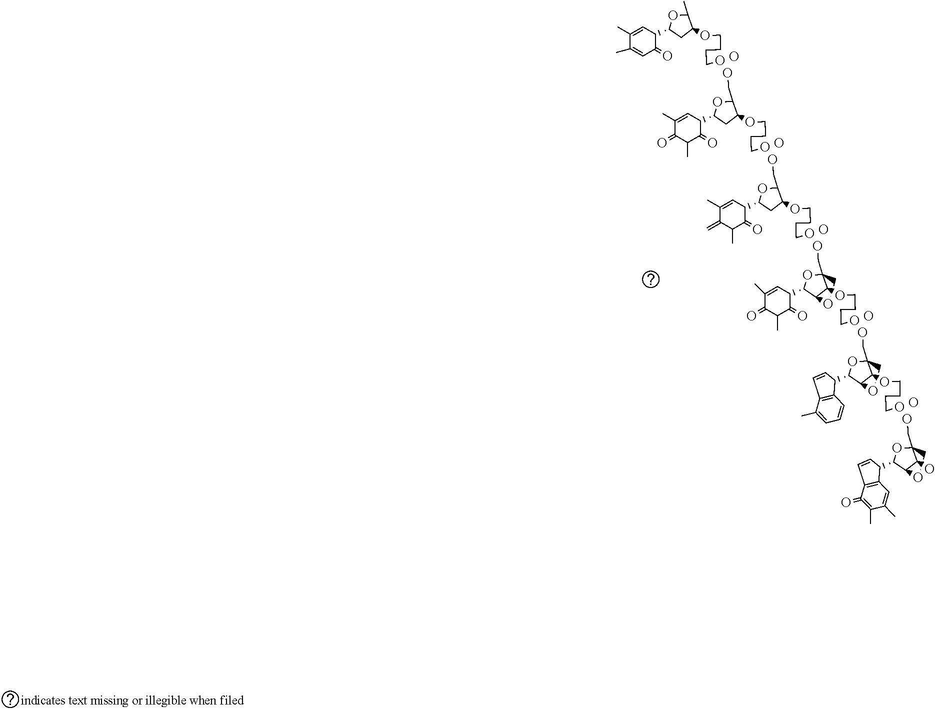

The compound was made according to Example 1 (C20-acid-MALAT1-LNA) starting from CamK2D ASO-hexylamine (40 mg, 7.24 μmol) and 22-((2,5-dioxopyrrolidin-1-yl)oxy)-22-oxodocosanoic acid (Intermediate 1) (6.77 mg, 14.5 μmol). Reaction time: 90 min. Purified on a XBridge C18, 5 μm 19×150 mm column by using a gradient from 15-90% methanol in NH₄HCO₃ (50 mM, pH8) at R.T. The yield was 8 mg (18%).

MS (ESI⁻) m/z 1469.2 (z=4)

### Example 13—Synthesis of C₂₂ Saturated Acid Conjugate of Camk2D Antisense Oligonucleotide (SEQ ID NO: 10)

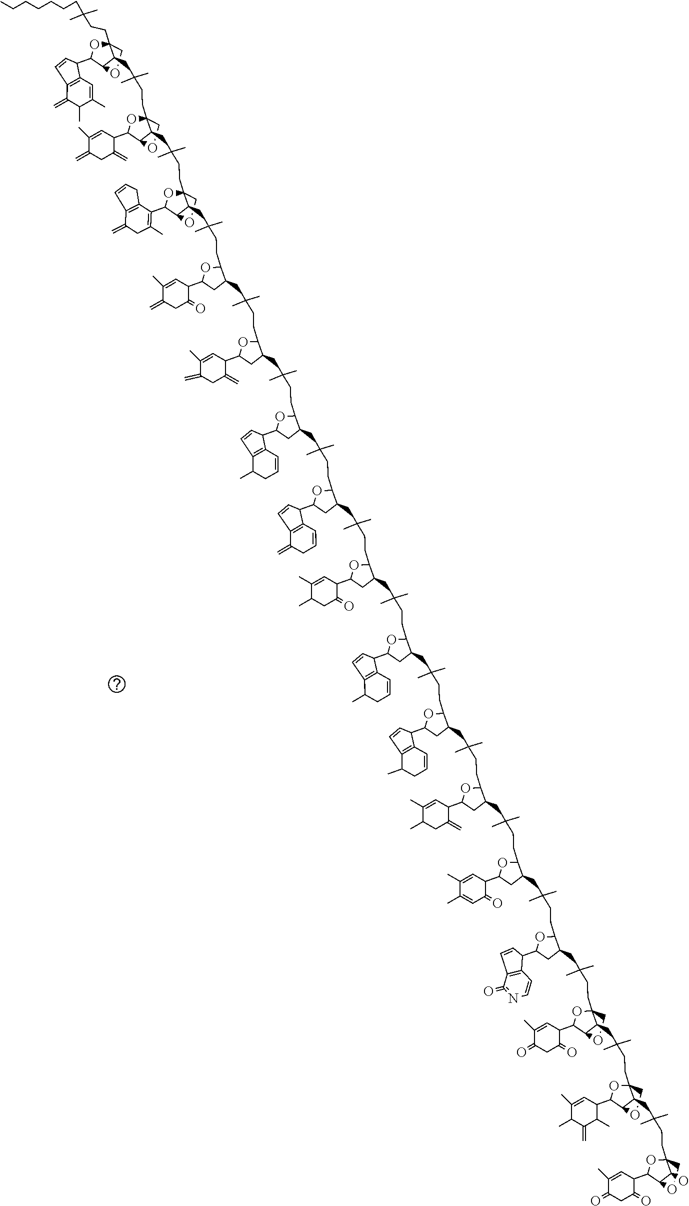

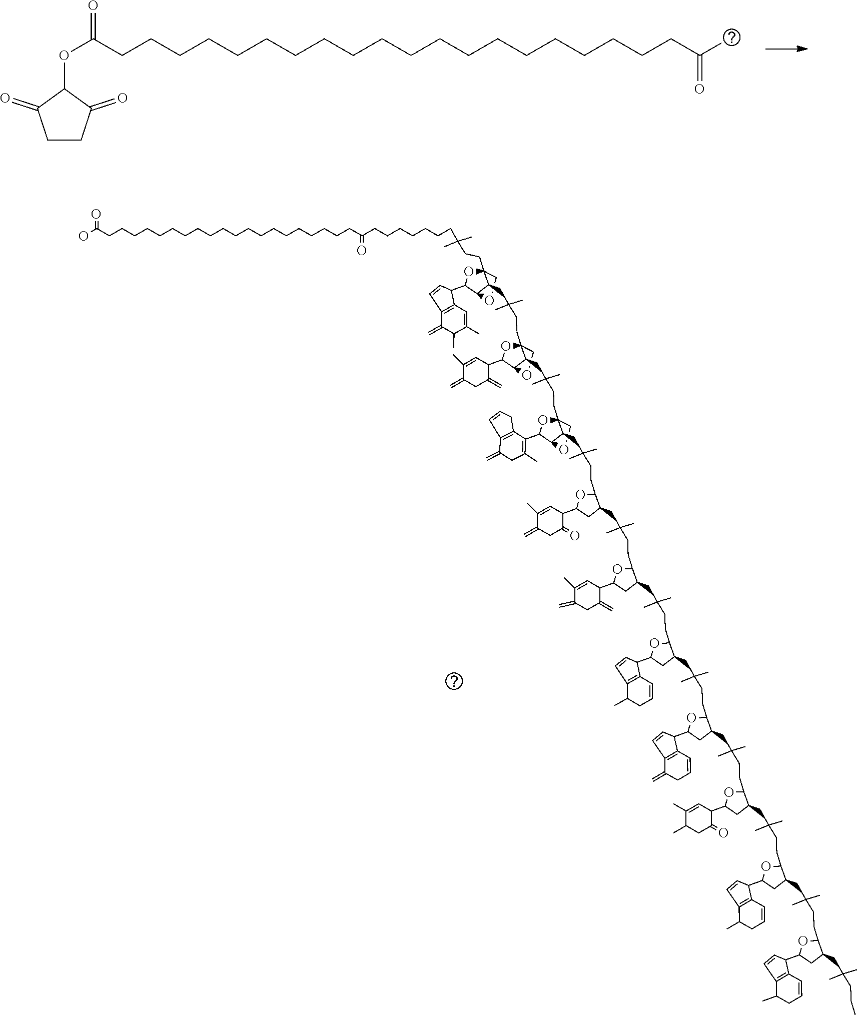

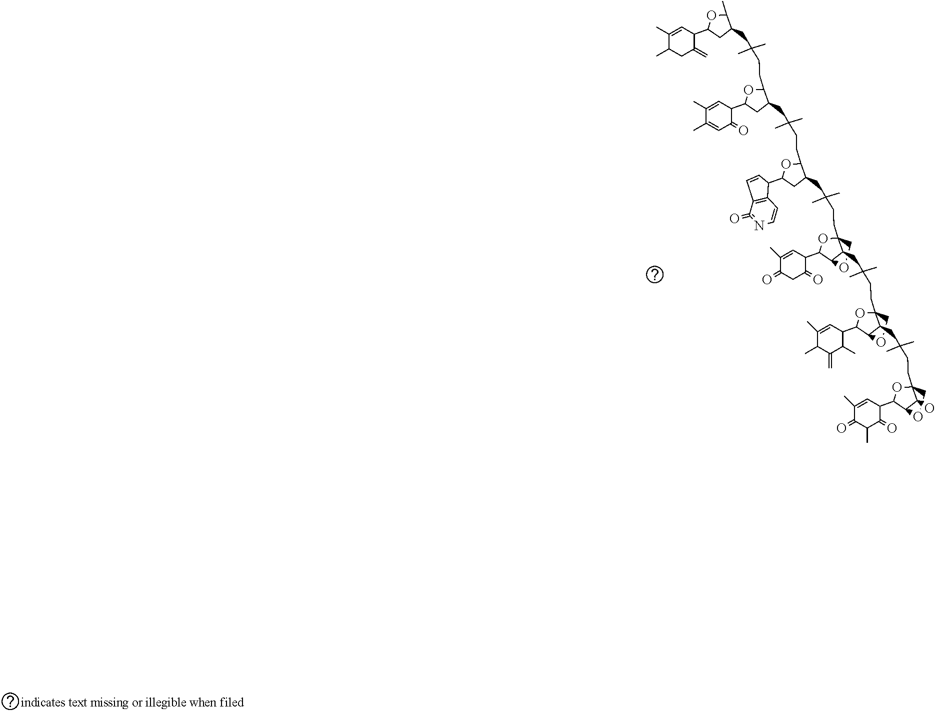

The compound was made according to Example 1 (C20-acid-MALAT1-LNA) starting from CamK2D ASO-hexylamine (40 mg, 7.24 μmol) and 22-((2,5-dioxopyrrolidin-1-yl)oxy)-22-oxodocosanoic acid (Intermediate 1) (6.77 mg, 14.5 μmol). Reaction time: 120 min. Purified on a XBridge C18, 5 μm 19×150 mm column by using a gradient from 10-90% acetonitrile in NH₄HCO₃ (50 mM, pH8) at R.T. The yield was 16 mg (36%).

MS (ESI⁻) m/z 1462.9 (z=4)

### Example 14—Synthesis of C₂₂ Saturated Acid Conjugate of Camk2D Antisense Oligonucleotide (SEQ ID NO: 11)

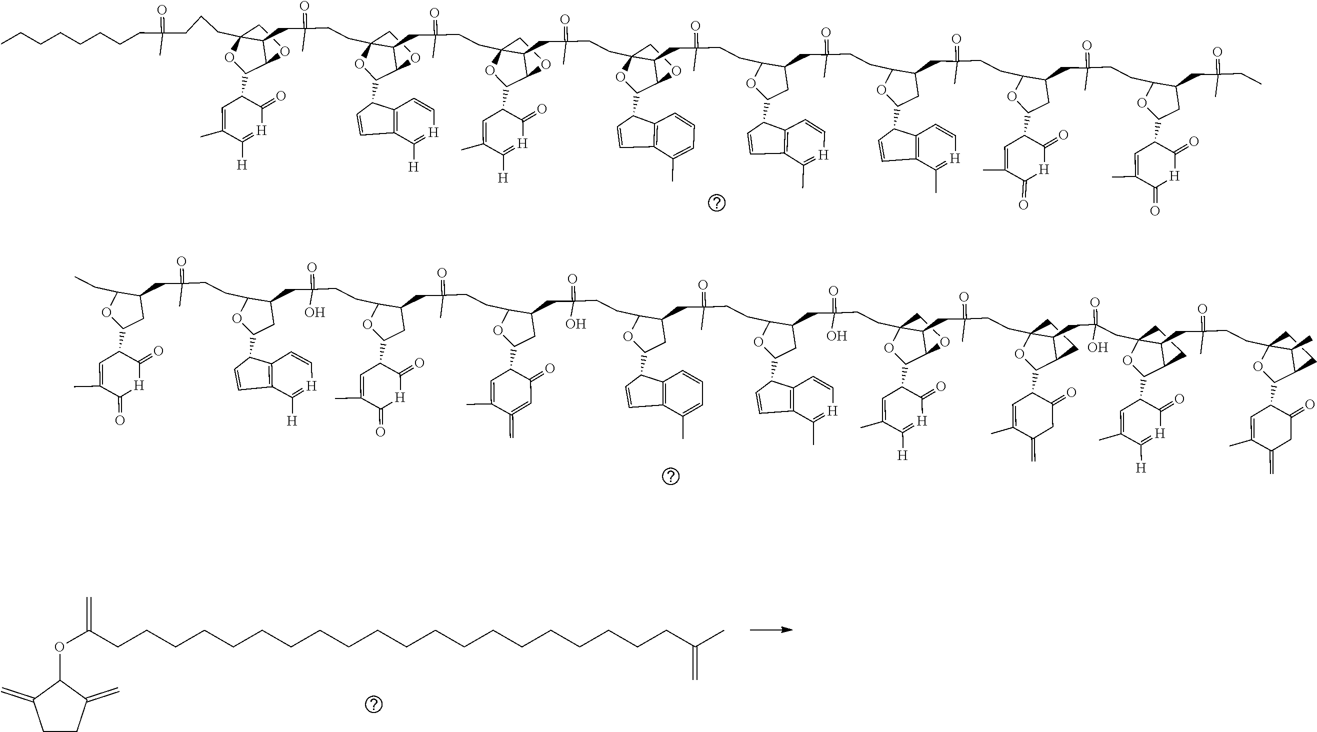

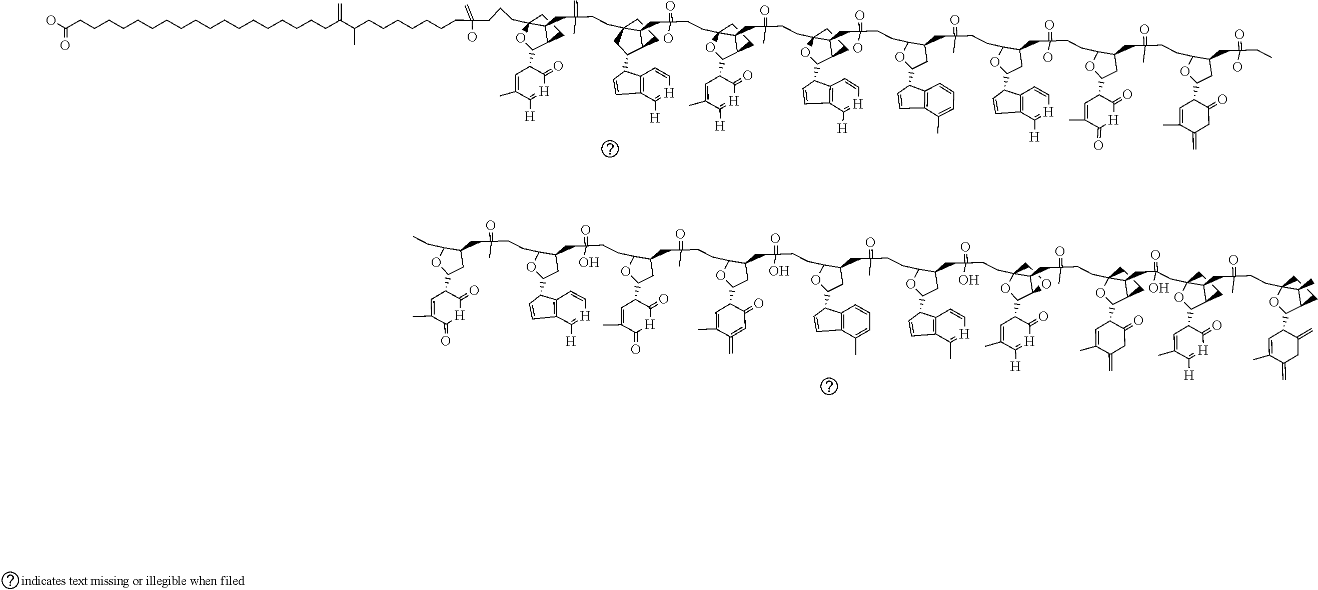

The compound was made according to Example 2 C22-acid-MALAT1-LNA starting from CamK2D ASO-hexylamine (35 mg, 5.68 μmol) and 22-((2,5-dioxopyrrolidin-1-yl)oxy)-22-oxodocosanoic acid (Intermediate 1) (5.31 mg, 11.4 μmol). Reaction time: 40 min. Purified on a XBridge C18, 5 μm 19×150 mm column by using a gradient from 10-50% acetonitrile 11 min, 50-90% acetonitrile in NH₄HCO₃ (50 mM, pH8) at R.T. The yield was 20.4 mg (52%).

MS (ESI⁻) m/z 1628.5 (z=4)

### Example 15—Synthesis of C₂₂ Saturated Acid Conjugate of Camk2D Antisense Oligonucleotide (SEQ ID NO: 12)

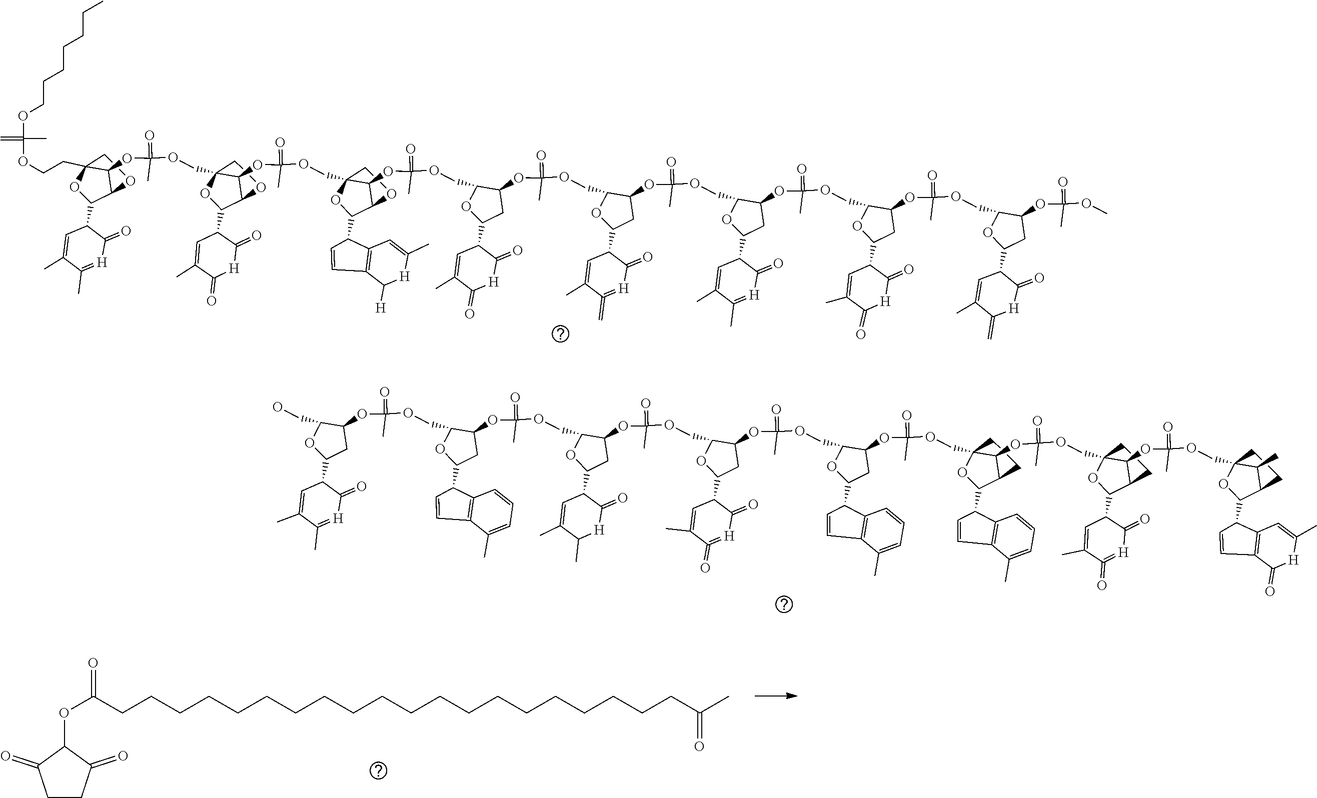

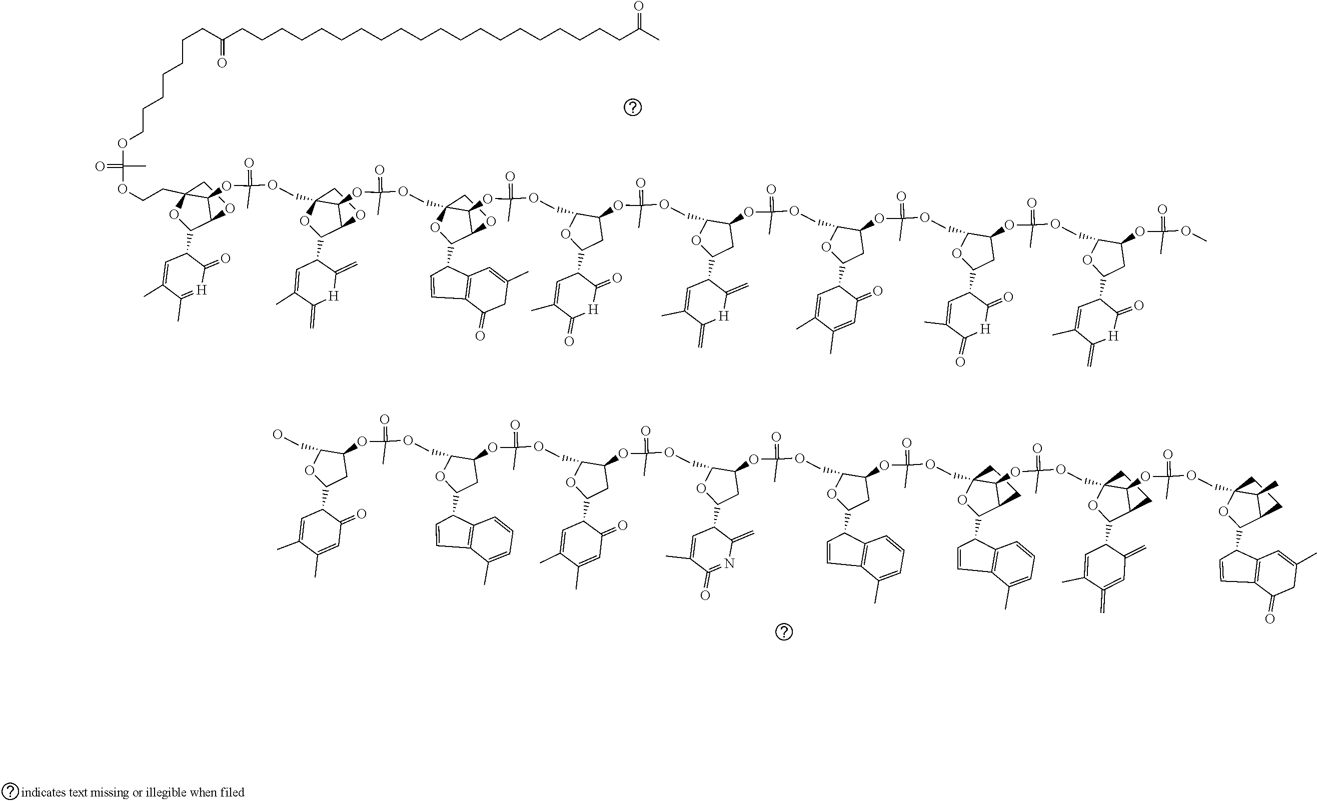

The compound was made according to Example 2 C22-acid-MALAT1-LNA starting from CamK2D ASO-hexylamine (35 mg, 5.68 μmol) and 22-((2,5-dioxopyrrolidin-1-yl)oxy)-22-oxodocosanoic acid (Intermediate 1) (5.31 mg, 11.4 μmol). Reaction time: 40 min. Purified on a XBridge C18, 5 μm 19×150 mm column by using a gradient from 10-50% AC 11 min, 50-90% ACN in NH₄HCO₃ (50 mM, pH8) at R.T. The yield was 20.4 mg (56%).

MS (ESI⁻) m/z 1458.4 (z=4)

### Example 16—Synthesis of C₂₂ Saturated Acid Conjugate of Camk2D Antisense Oligonucleotide (SEQ ID NO: 13)

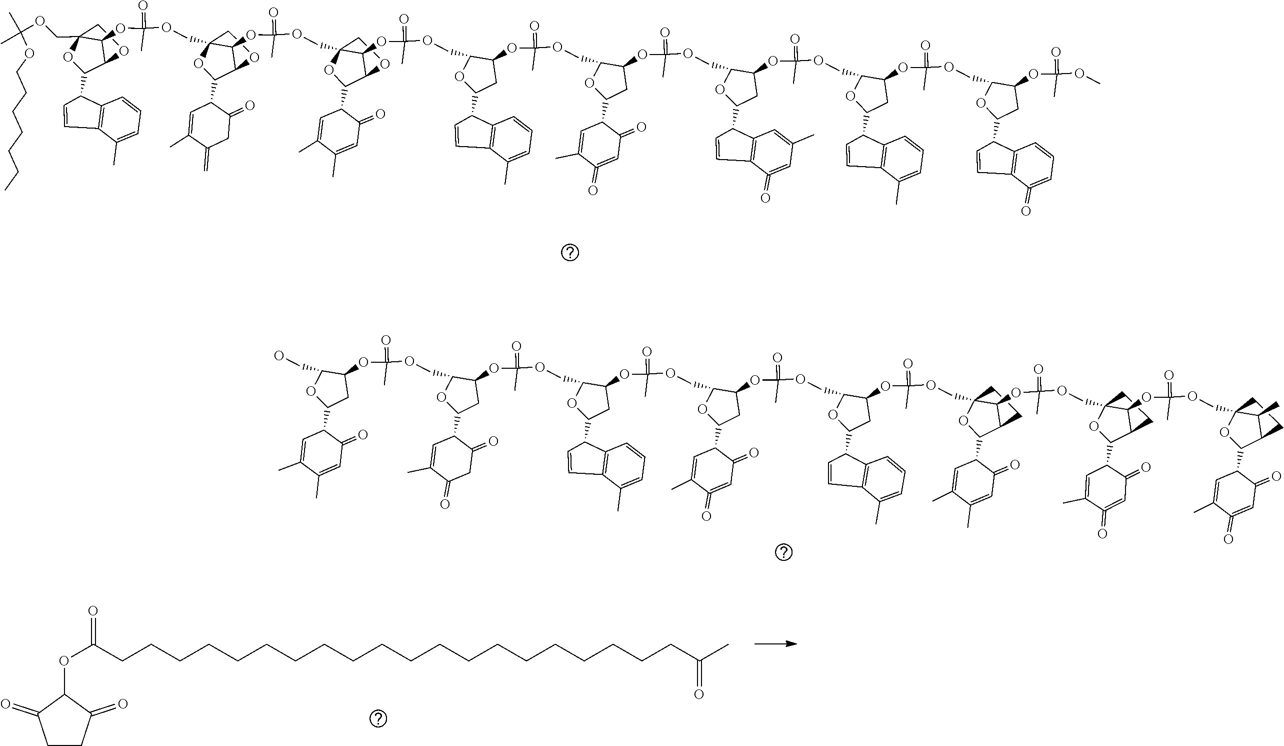

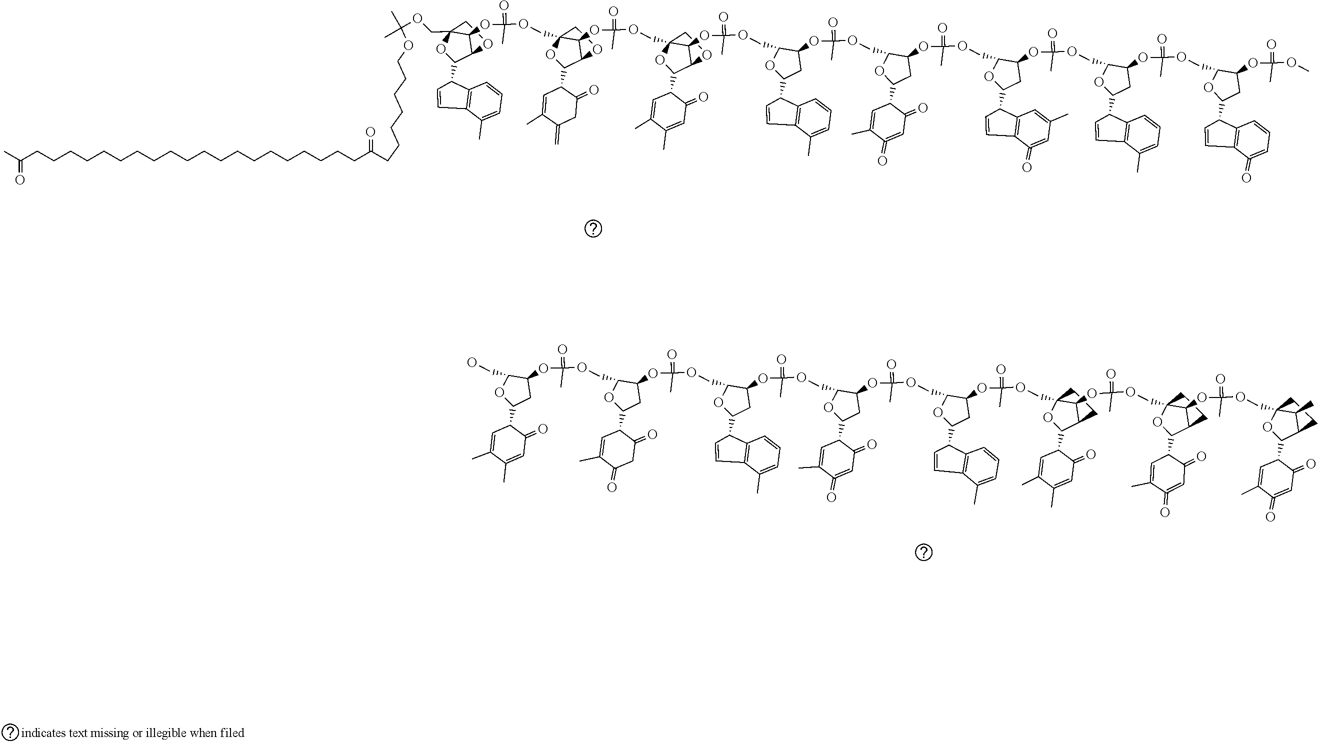

The compound was made according to Example 2 C22-acid-MALAT1-LNA starting from CamK2D ASO-hexylamine (35 mg, 5.68 μmol) and 22-((2,5-dioxopyrrolidin-1-yl)oxy)-22-oxodocosanoic acid (Intermediate 1) (5.31 mg, 11.4 μmol). Reaction time: 40 min. Purified on a XBridge C18, 5 μm 19×150 mm column by using a gradient from 10-50% acetonitrile 11 min, 50-90% acetonitrile in NH₄HCO₃ (50 mM, pH8) at R.T. The yield was 18.5 mg (53%).

MS (ESI⁻) m/z 1462.6 (z=4)

### Example 17—Synthesis of C₂₂ Saturated Acid Conjugate of PEG-4 Malat-1 LNA Antisense Oligonucleotide (SEQ ID NO: 8)

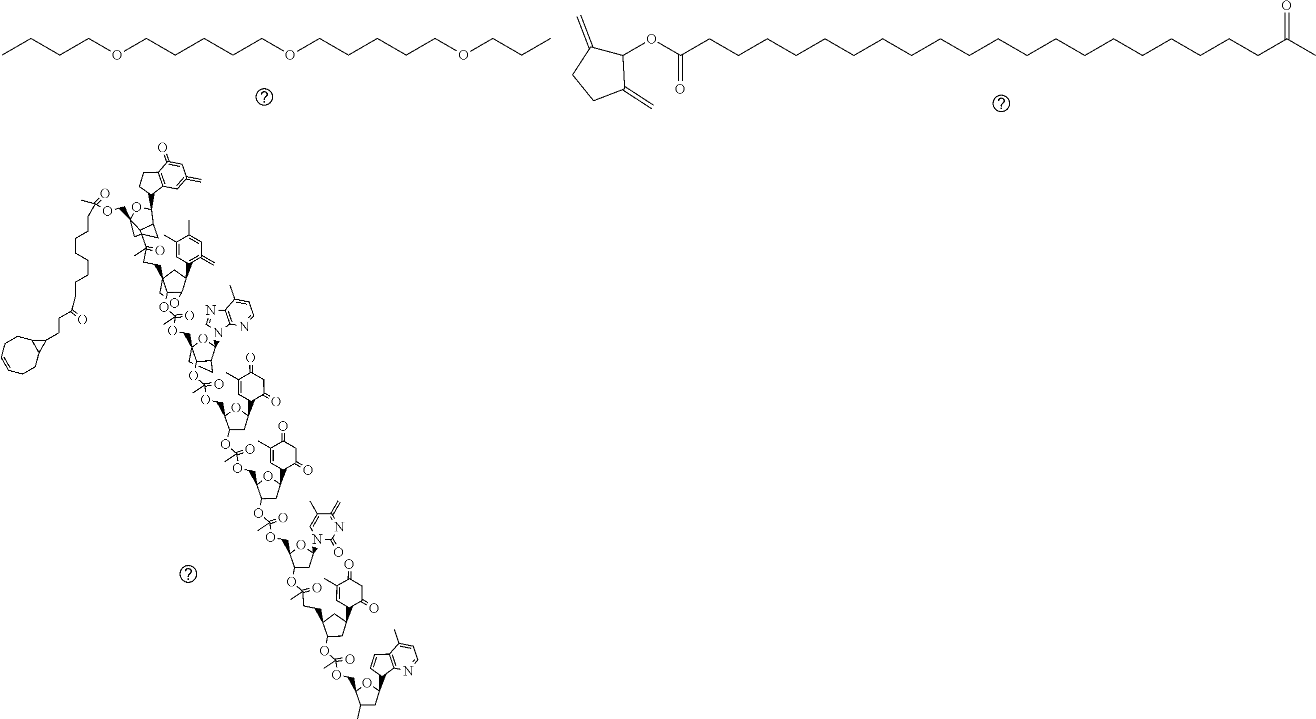

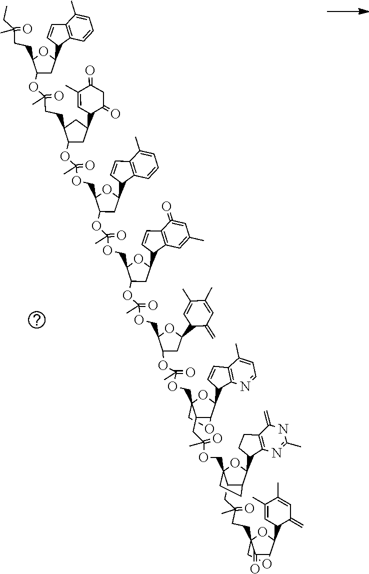

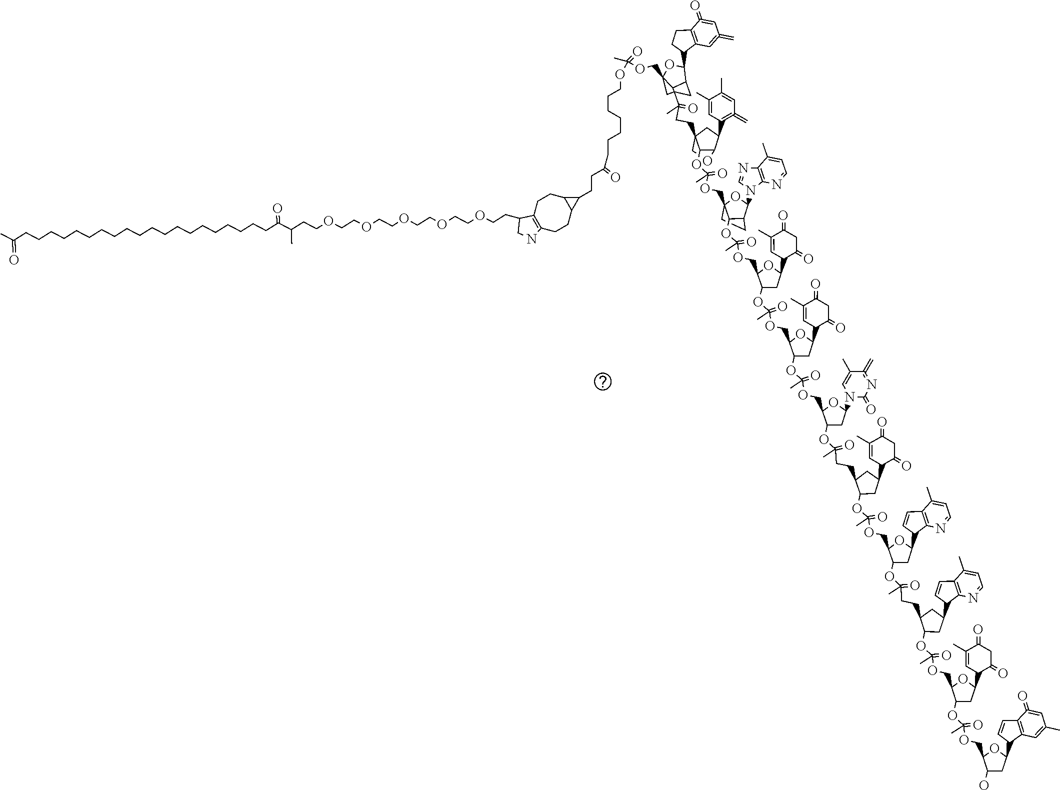

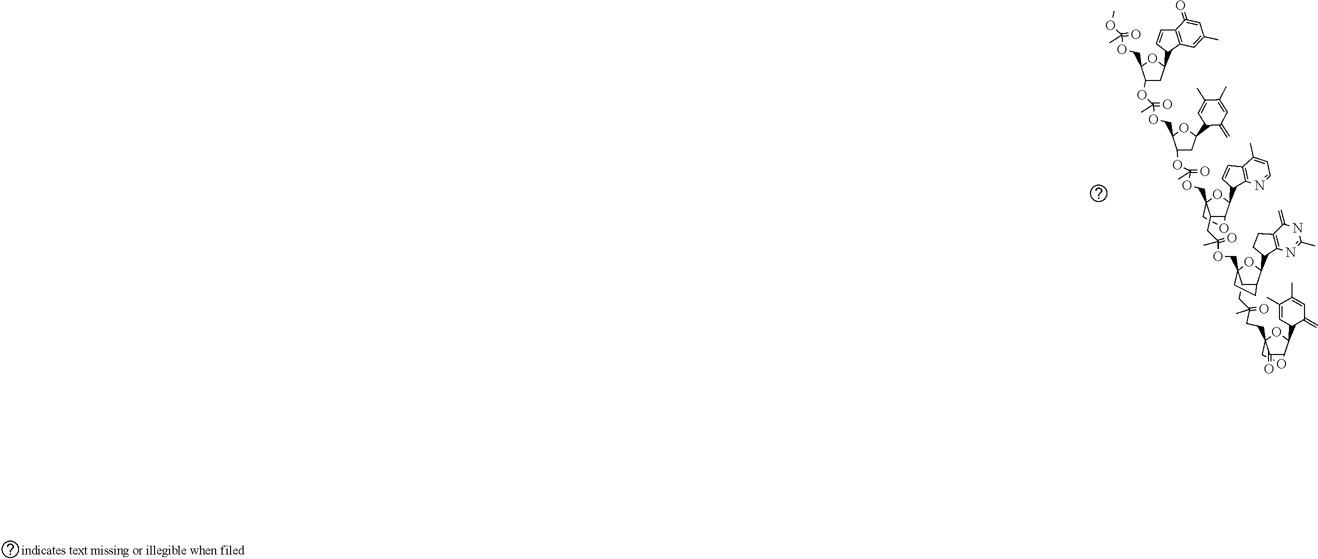

To Intermediate 14 (20 mg, 3.5 μmol) dissolved in water (600 μl) was added a solution of 17-azido-3,6,9,12,15-pentaoxaheptadecan-1-amine in acetonitrile (150 μl) and DMSO (90 μl). Stirred at rt for 30 min. To the reaction mixture was added triethylamine (5.83 μl). The mixture was heated to 45° C. and a warm solution of 22-((2,5-dioxopyrrolidin-1-yl)oxy)-22-oxodocosanoic acid (Intermediate 1) (5 mg, 10.5 μmol) was added. The mixture was stirred at 45° C. for 30 min. Sodium acetate 3M, pH 5.2 (500 μl) was added and then the oligo was precipitated by addition of ethanol (24 ml). The mixture was centrifuged at 0° C. for 10 mins at 3500 rpm and the clear supernatant was removed. The residue was washed with EtOH (5 ml.) and the pellet was dried under vacuum. The residue was purified on a XBridge C18, 5 μm 19×150 mm column by using a gradient from 15-60% acetonitrile 11 min, 60-90% acetonitrile 3 min in NH₄HCO₃ (50 mM, pH8) at R. T. The pure fractions were freeze-dried twice. Yield. 9.8 mg (42%).

MS (ESI⁻) m/z 1589.9 (z=4)

### Example 18—Synthesis of Bilipid C₂₂ Saturated Acid Conjugate of PEG-4 Malat-1 LNA Antisense Oligonucleotide (SEQ ID NO: 8)

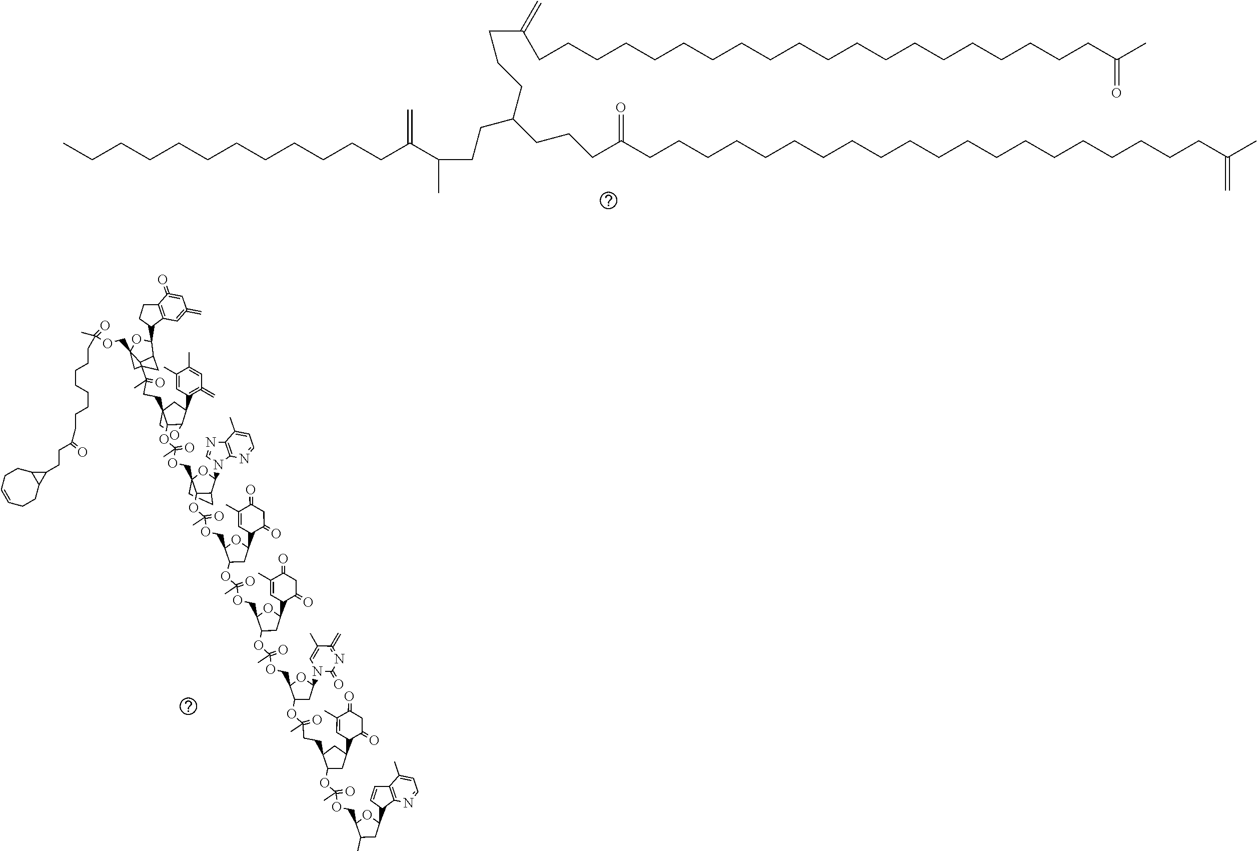

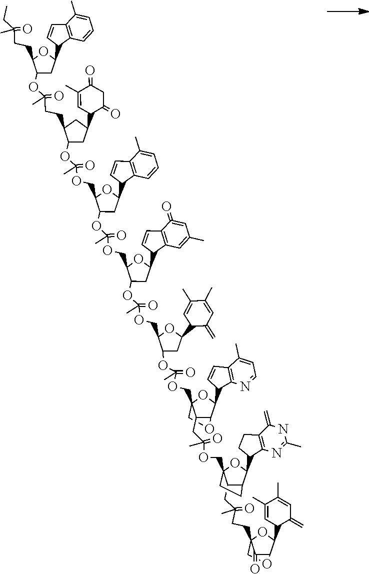

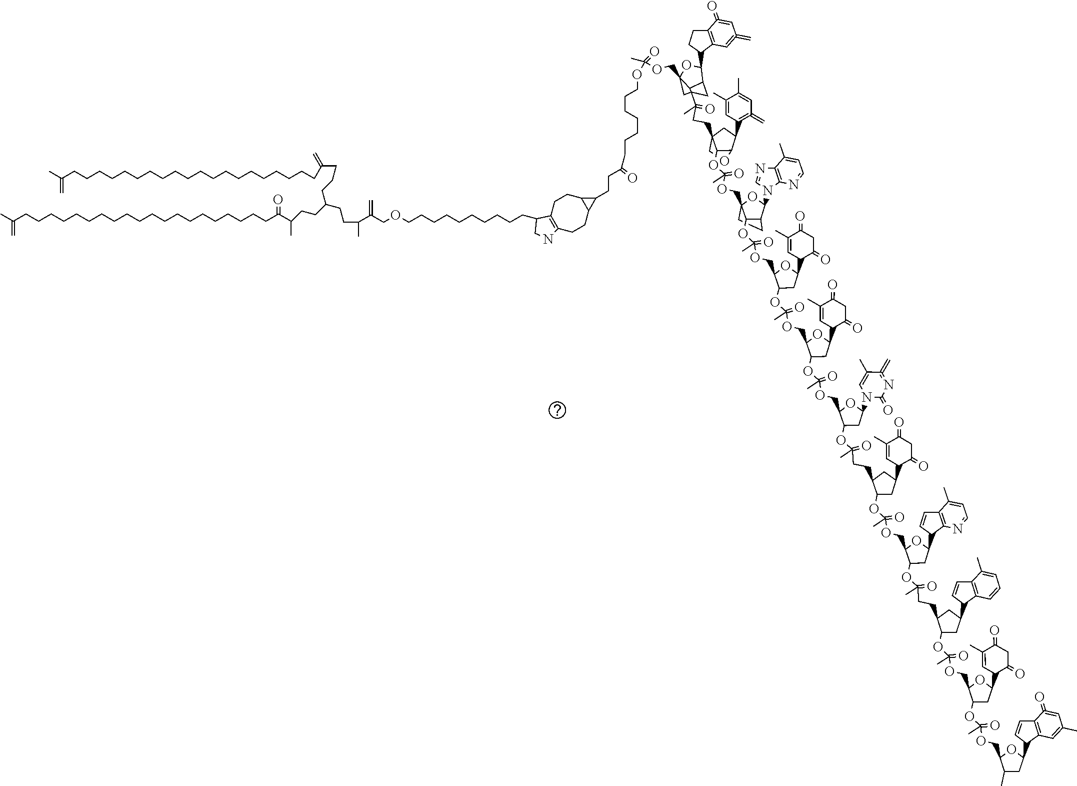

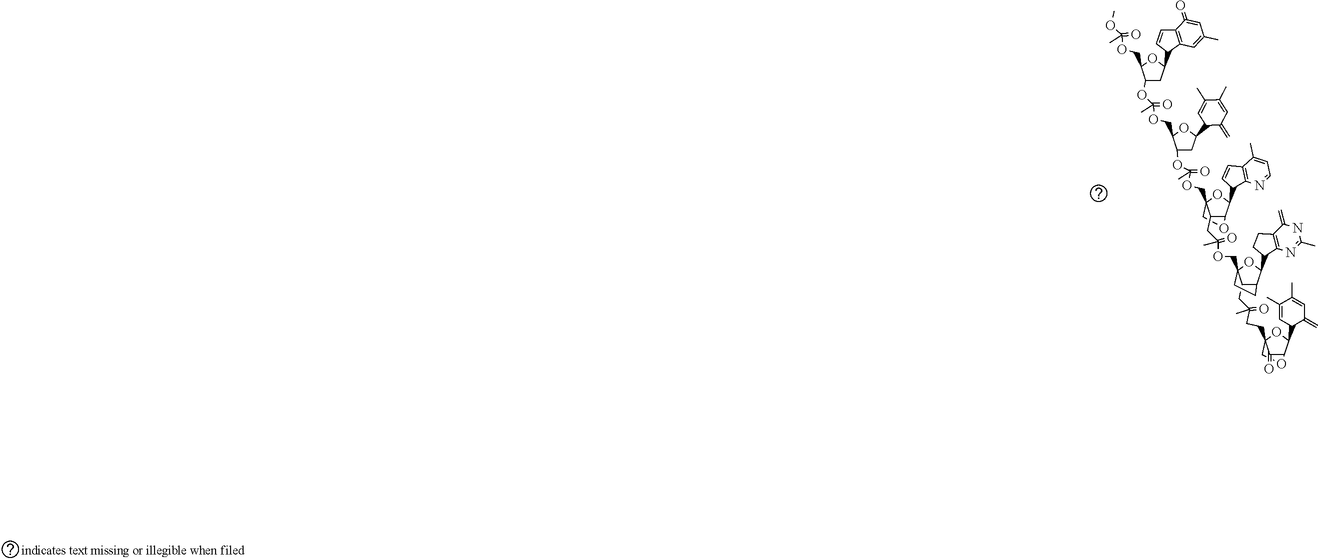

To a warm solution of Intermediate 16 (4.3 mg, 4.05 μmol) in water (200 μl) and THF (100 μl) was added a warm solution of Intermediate 14 (21 mg, 3.68 μmol) in water (300 μl) and tetrahydrofuran (200 μl). The mixture was stirred at 45° C. for 3 h. Sodium acetate 3M, pH 5.2 (500 μl) was added and then the oligo was precipitated by addition of ethanol (24 ml). The mixture was centrifuged at 0° C. for 10 mins at 3500 rpm and the clear supernatant was removed. The residue was washed with EtOH (5 ml.) and the pellet was dried under vacuum. The residue was purified on a XBridge C18, 5 μm 19×150 mm column by using a gradient from 20-90% acetonitrile in NH₄HCO₃ (50 mM, pH8) at R.T. The pure fractions were evaporated to ⅔ of the volume and freeze-dried twice. Yield: 3 mg (11%).

MS (ESI⁻) m/z 1691.3 (z=4)

### Example 19—Synthesis of C₂₂ Saturated Acid Conjugate of Open Maleimide Malat-1 LNA Antisense Oligonucleotide (SEQ ID NO: 8)

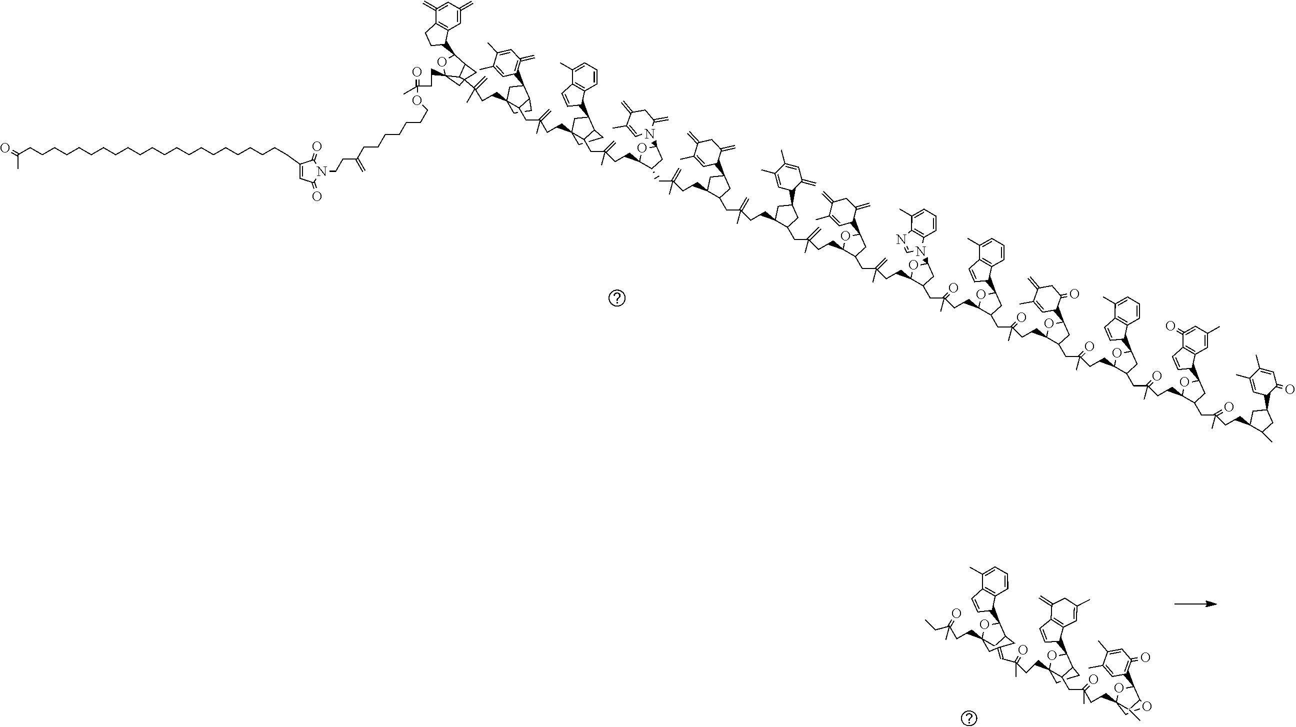

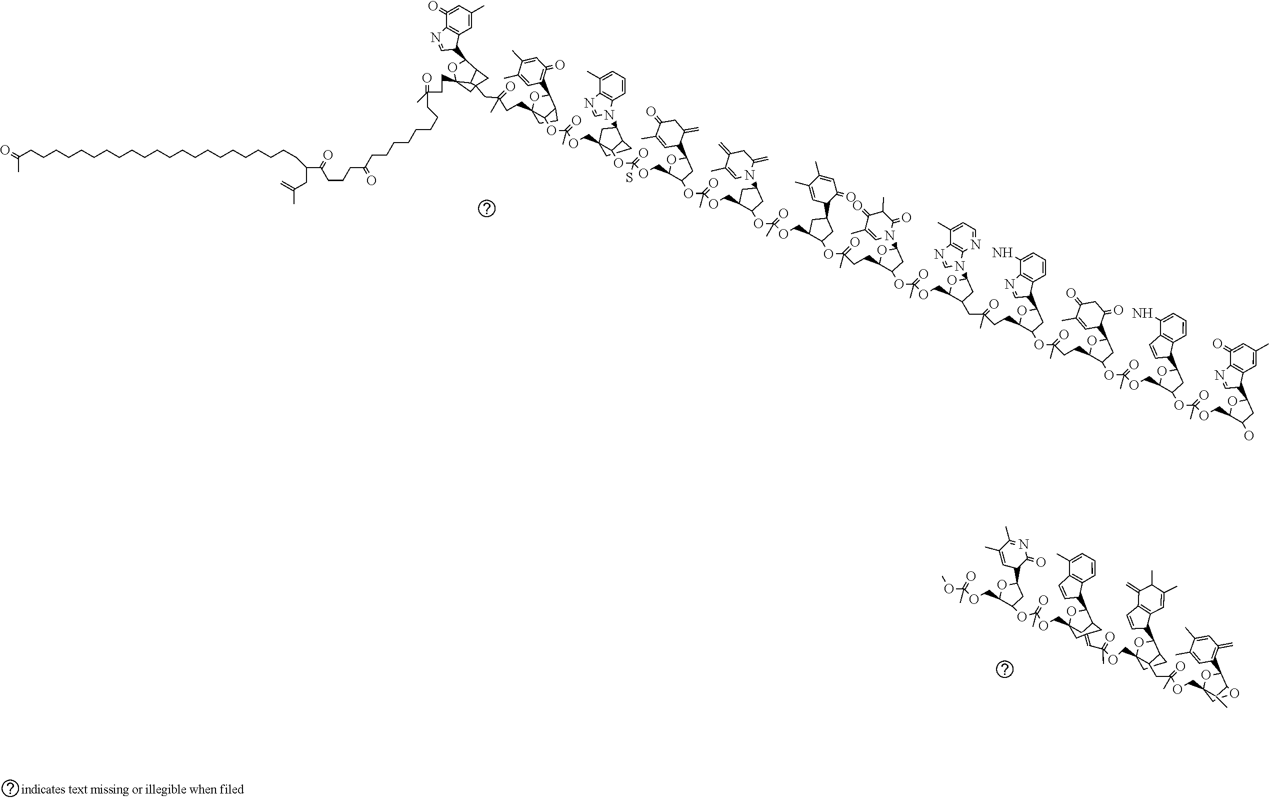

To C22-acid Maleimide-MALAT1-LNA (40 mg, 6.31 μmol) was added a saturated water solution of sodium hydrogen carbonate (2 ml), water (0.6 mL) and acetonitrile (0.600 ml). The mixture was stirred at rt for 3 h. Sodium hydroxide (1M, 150 μl, 150 μmol). The reaction was stirred at 30° C. for 17 h then at 40° C. for 10 h. The mixture was cooled in ice and pH was adjusted to pH5 with acetic acid. The milky solution was filtered and freeze dried. The residue was purified on a XBridge C18, 5 μm 19×150 mm column by using a gradient from 10-90% acetonitrile in NH₄HCO₃ (50 mM, pH8) at R.T. The pure fractions were freeze-dried twice. Yield: 13 mg (32.2%).

MS (ESI⁻) m/z 1516.1 (z=4)

### Example 20—Synthesis of C₂₂ Saturated Acid Conjugate of Cysteine Maleimide Malat-1 LNA Antisense Oligonucleotide (SEQ ID NO: 8)

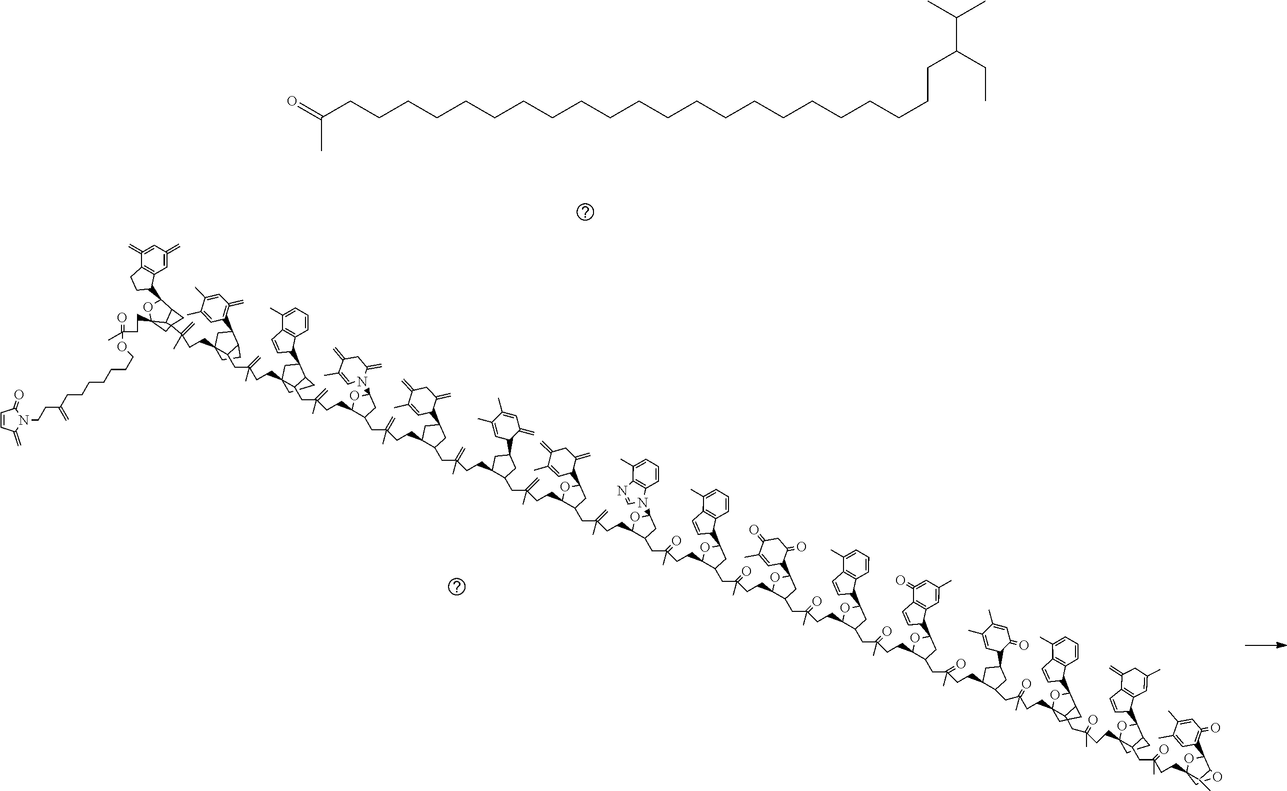

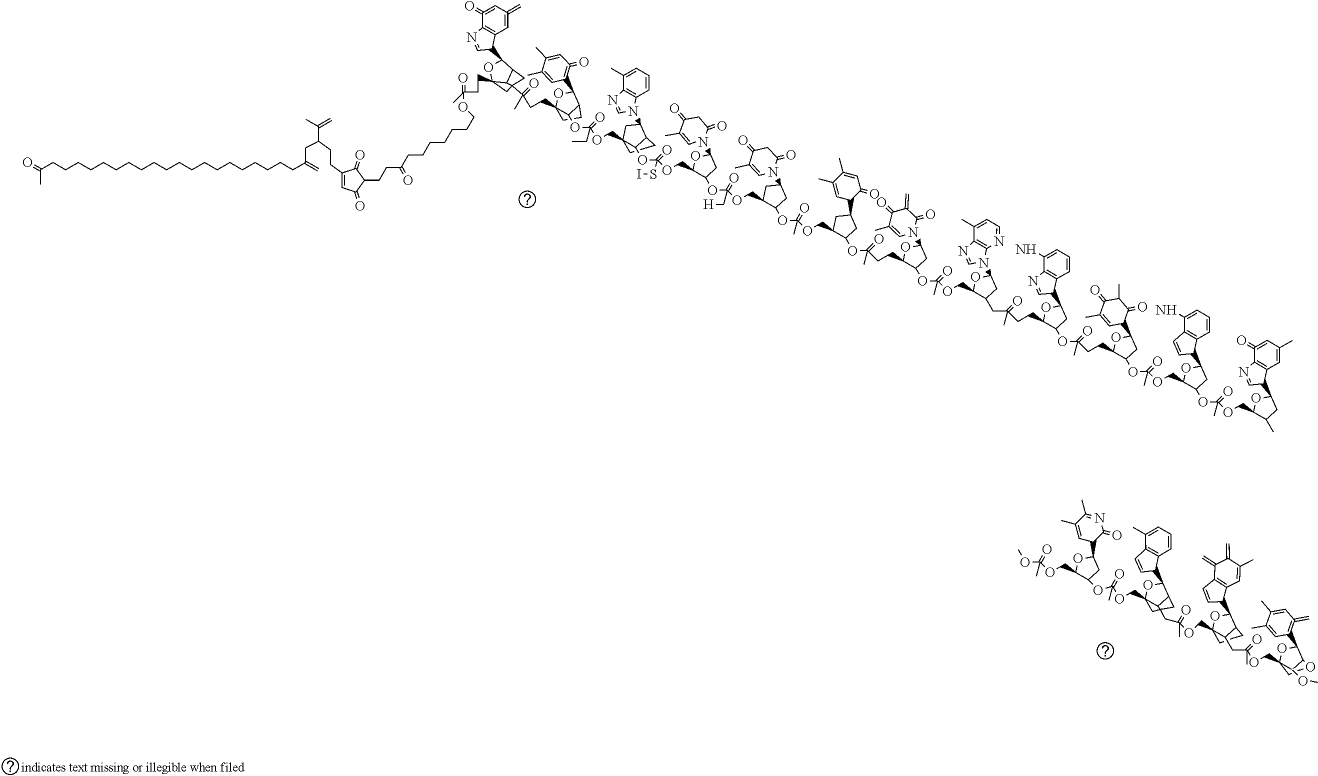

Intermediate 19 (6.8 mg, 7.2 μmol) was treated with 3,3′,3″-phosphanetriyltripropionic acid hydrochloride (4.12 mg, 10 μmol) in a mixture of tetrahydrofuran (300 μl), sodium acetate buffer (pH 5.3, 3M, 50.0 μl) and water (100 μl) at 40° C. for 10 mins. The mixture was diluted with water and the sulphide was extracted with dichloromethane. The organic phase was evaporated and the desired sulphide (3 mg, 6.33 μmol) was dissolved in dimethyl formamide (0.200 mL) and added to Intermediate 17 (Maleimide-MALAT1-LNA (25 mg, 4.40 μmol) in sodium acetate buffer (3M, pH5.2, 100 μl), water (500 μl) and dimethyl formamide (200 μl). The mixture was heated at 45° C. for 2 h. Acetonitrile (100 μl) and another portion of sulphide (3 mg, 6.33 μmol) dissolved in dimethyl formamide (0.200 mL) was added and heating was continued for another 1 h. The oligo was precipitated by addition of ethanol (14 mL), vortexed briefly and left standing at −20 C for 30 min. The mixture was centrifuged at 0° C. for 10 mins at 3500 rpm and the clear supernatant was removed. The oligo was washed once more by dissolving the pellet in 1 mL of water and Sodium acetate 3M, pH 5.2 (100 μl) followed by addition of 14 mL of ethanol, cooled for 30 mins at −20, then centrifuged at 0° C. for 10 mins at 3500 rpm. The supernatant was discarded, and the product was dried in vacuum. The residue was purified on a XBridge C18, 5 μm 19×150 mm column by using a gradient from 5-90% acetonitrile in NH₄HCO₃ (50 mM, pH8) at R.T. The pure fractions were freeze-dried twice. Yield: 7.5 mg (26.4%).

MS (ESI⁻) m/z 1536.7 (z=4)

### Example 21—Synthesis of C₂₂ Saturated Acid Conjugate of PEG Cysteine Maleimide Malat-1 LNA Antisense Oligonucleotide (SEQ ID NO: 8)

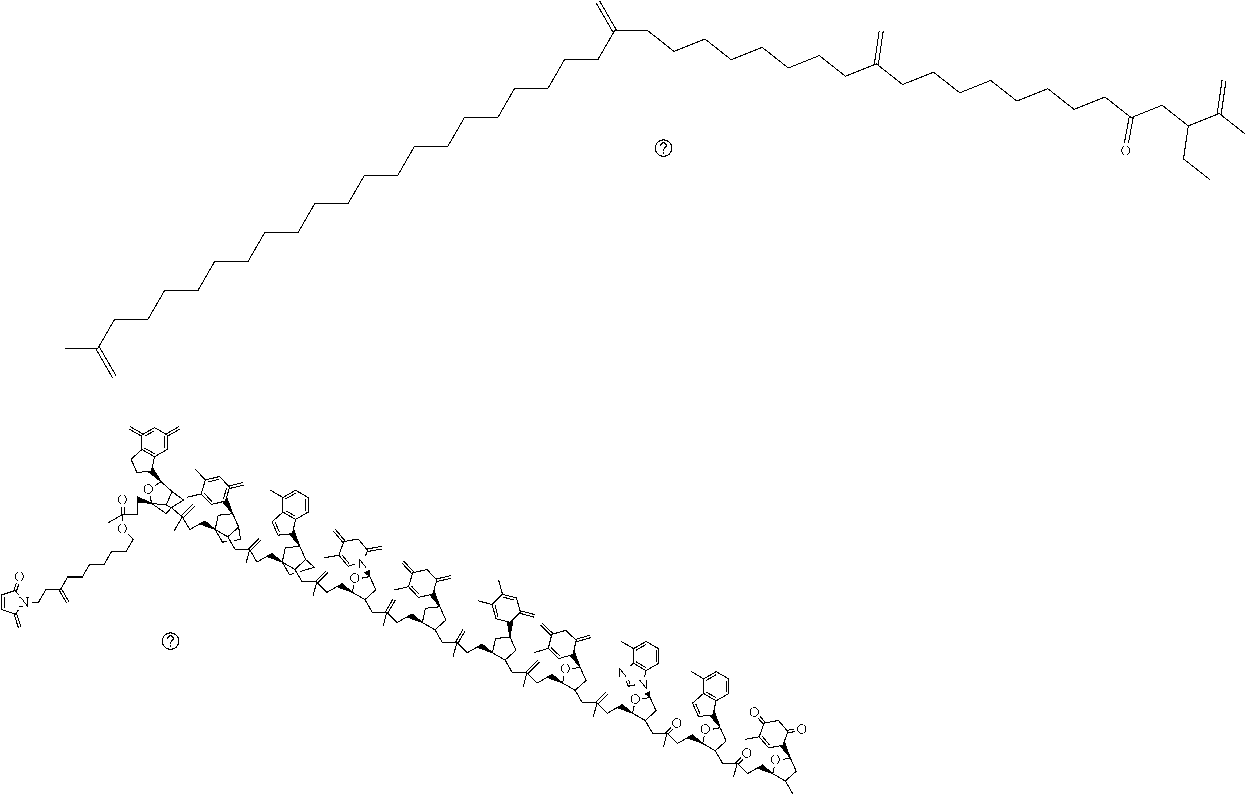

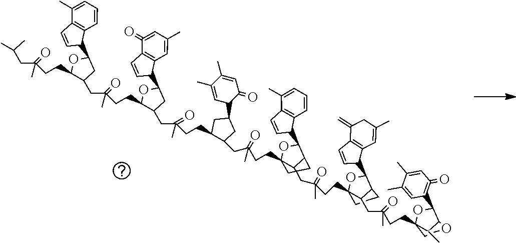

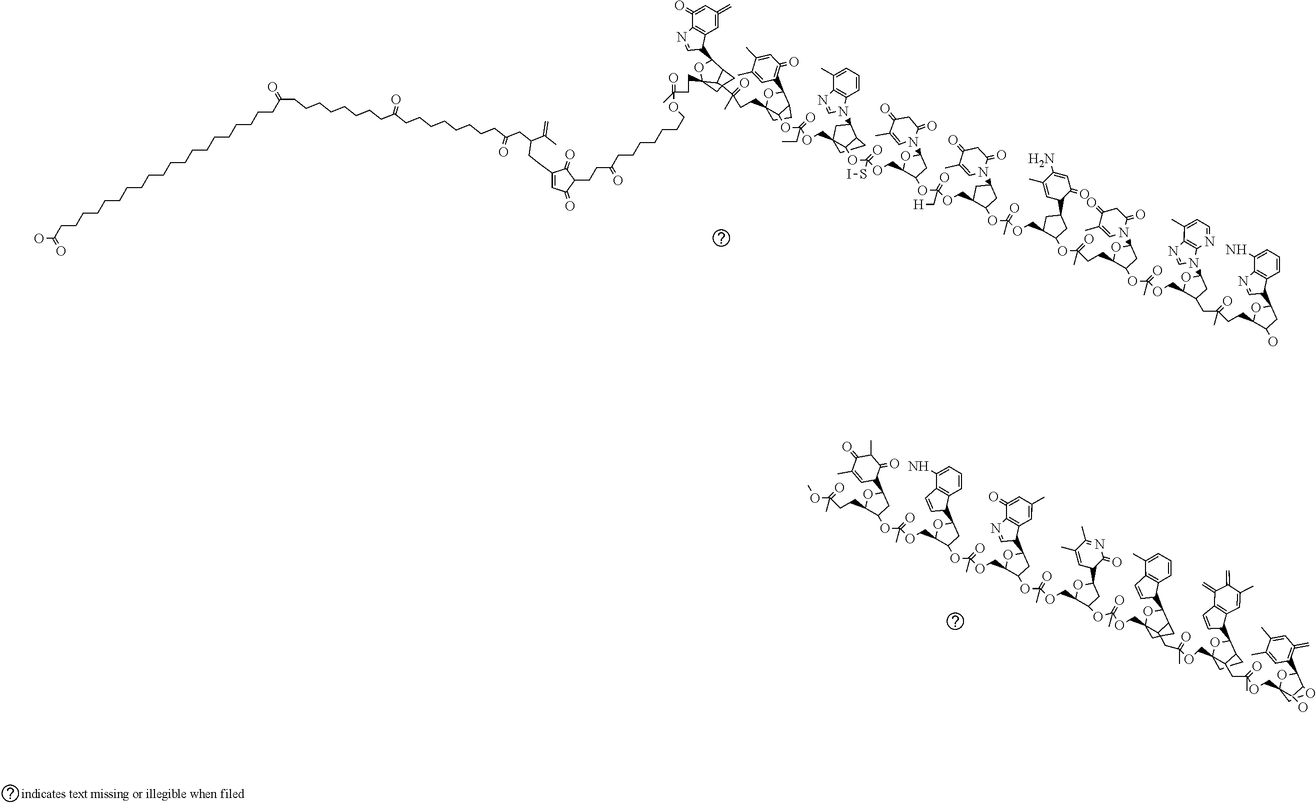

To a solution of Intermediate 17 (Maleimide-MALAT1-LNA (30 mg, 5.28 μmol) in water (1 ml), was added Intermediate 21 (1-{[(2R)-1-amino-1-oxo-3-sulfanylpropan-2-yl]amino}-1,10,19-trioxo-3,6,12,15-tetraoxa-9,18-diazatetracontan-40-oic acid) (12.1 mg, 20 μmol) as a solution in dimethylformamide (1.5 ml). The mixture was heated at 45° C. for 30 min. Water (1 ml) was added, resulting in some precipitation. The mixture was extracted with diethylether/tetrahydofuran (0.5 ml) to remove excess of Intermediate 21. To the water phase was then added ethanol (14 mL) to precipitate the oligo. The mixture was vortexed briefly and left standing at −20 C for 30 min, centrifuged at 0° C. for 10 mins at 3500 rpm and the clear supernatant was removed. The oligo was washed once more by dissolving the pellet in 1 mL of water and adding sodium acetate 3M, pH 5.2 (100 μl) followed by addition of 14 mL of ethanol, cooled for 30 mins at −20, then centrifuged at 0° C. for 10 mins at 3500 rpm. The supernatant was discarded and the product was dried in vacuum. The residue was purified on a XBridge C18, 5 μm 19×150 mm column by using a gradient from 10-90% acetonitrile in NH₄HCO₃ (50 mM, pH8) at R. T. The pure fractions were freeze-dried twice. Yield: 11.5 mg (32.3%).

MS (ESI⁻) m/z 1609.0 (z=4)

### Example 22—Synthesis of C₂₂ Saturated Acid Conjugate of Maleimide Malat-1 LNA Antisense Oligonucleotide (SEQ ID NO: 8)

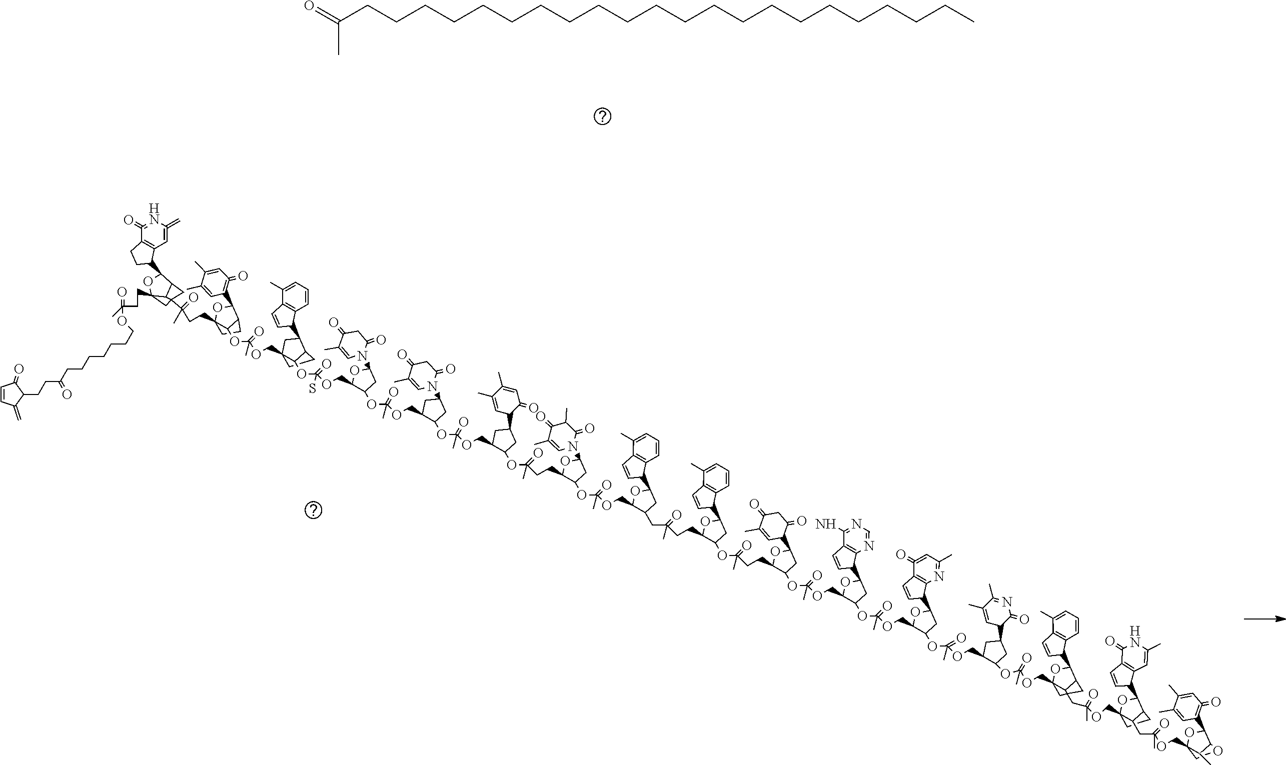

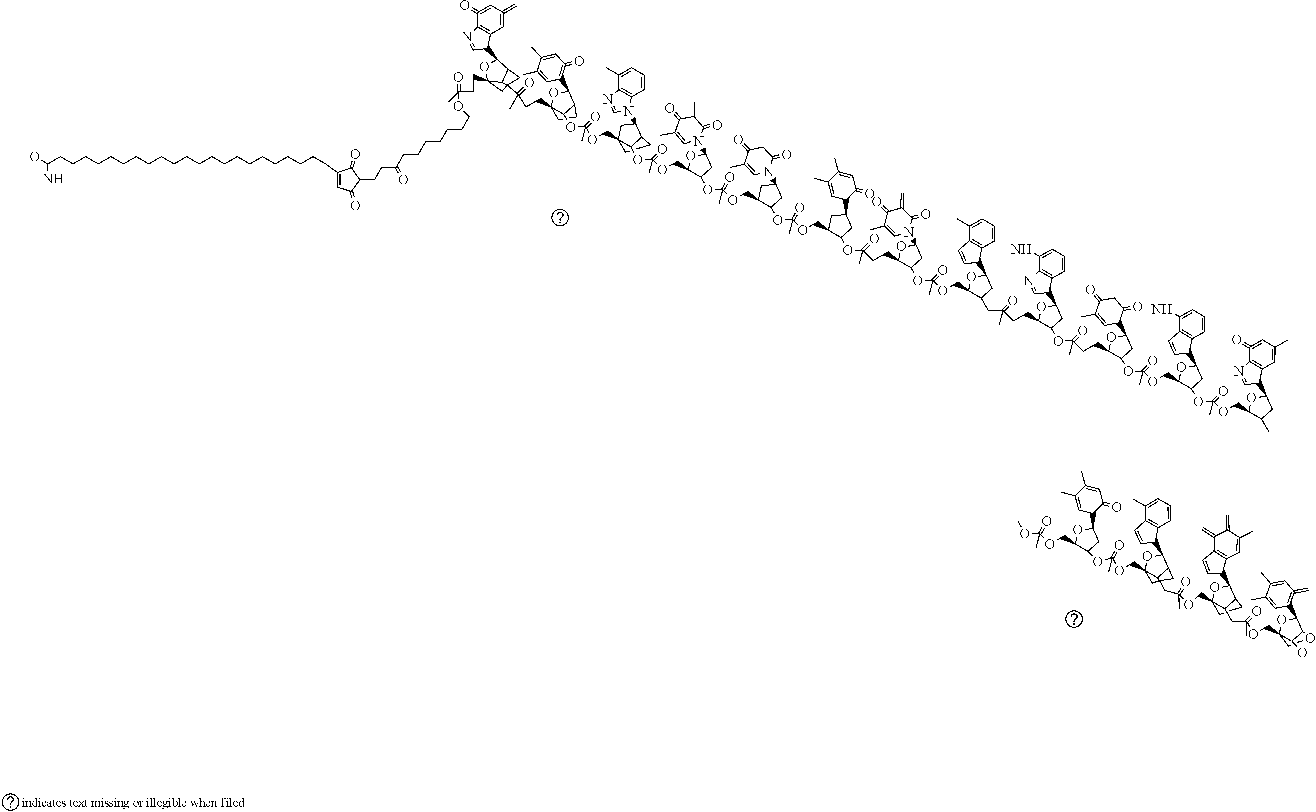

The compound was made according to C22-acid Cystein Maleimide-MALAT1-LNA starting from Intermediate 17 (Maleimide-MALAT1-LNA (10 mg, 1.76 μmol) and 22-mercaptodocosanoic acid (2.00 mg, 5.28 μmol) published compound JACS 2003 (125) p 7704-7714 Khoshtariya, Dimitri et al) The yield was 2.7 mg (24.2%).

MS (ESI⁻) m/z 1511.3 (z=4)

### Example 23—Synthesis of C₂₂ Saturated Acid Conjugate of LYS-PEG Squaric Amide Malat-1 LNA Antisense Oligonucleotide (SEQ ID NO: 8)

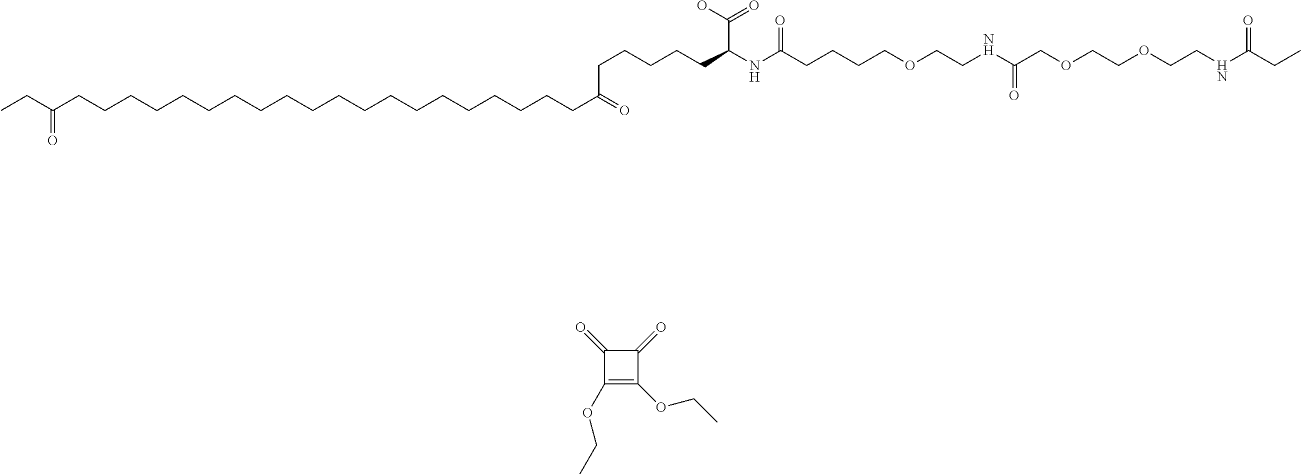

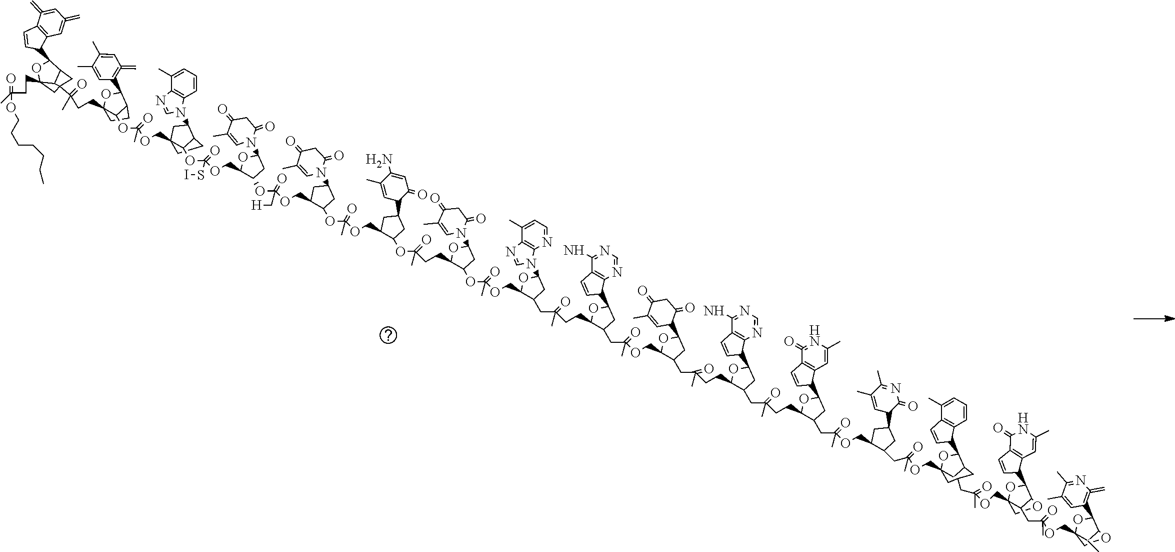

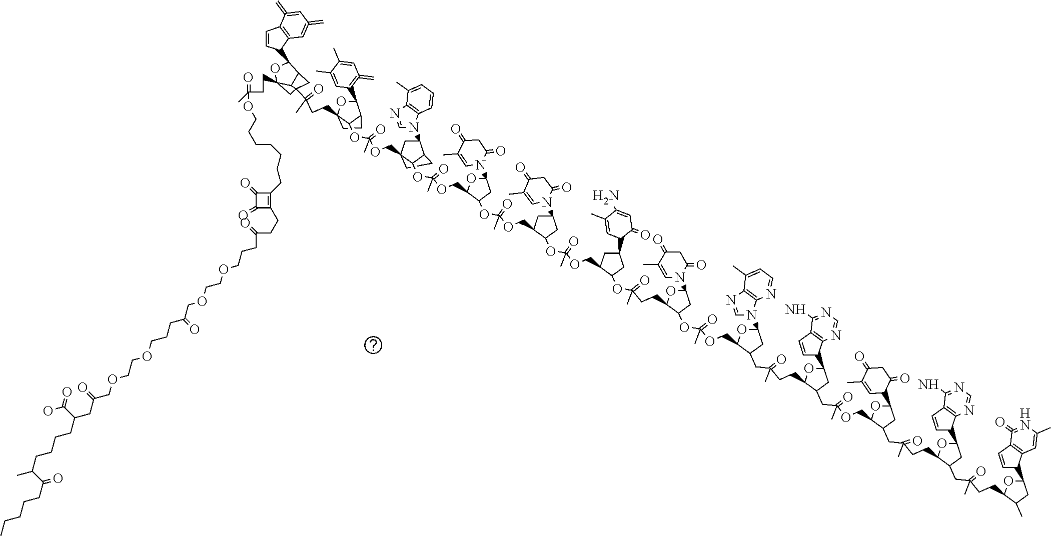

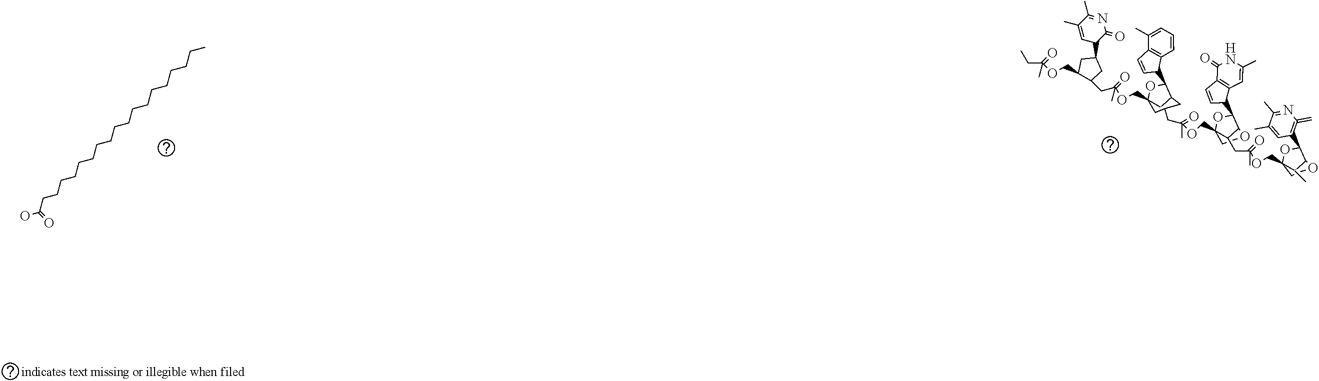

To MALAT1-LNA-hexylamine (27 mg, 4.89 μmol) in sodium hydrogen carbonate (pH 10.0, 0.025M, 600 μL) and acetonitrile (200 μL) was added a solution of 3,4-diethoxycyclobut-3-ene-1,2-dione (1.1 mg, 6.35 μmol) in acetonitrile (127 μl). The mixture was stirred at rt for 30 min. Another portion of 3,4-diethoxycyclobut-3-ene-1,2-dione (1.1 mg, 6.35 μmol) in acetonitrile (127 μl) was then added for complete reaction. After stirring for 30 min, a solution of Intermediate 22 (S)-1-amino-22-carboxy-2,11,20,28-tetraoxo-6,9,15,18-tetraoxa-3,12,21,27-tetraazanonatetracontan-49-oic acid (20.67 mg, 0.02 mmol) in DMSO (300 μL) and sodium bicarbonate, 0.15M (489 μl, 0.07 mmol) was then added and the reaction was heated at 40° C. for 18 h. The product was purified on a XBridge C18, 5 μm 19×150 mm column by using a gradient from 10-90% acetonitrile in NH₄HCO₃ (50 mM, pH8) at R. T. The pure fractions were freeze-dried twice. Yield: 8.5 mg (25.7%).

MS (ESI⁻) m/z 1611.4 (z=4)

### Example 24—Synthesis of C₂₂ Saturated Acid Conjugate of gamma GLU-PEG Maleimide Malat-1 LNA Antisense Oligonucleotide (SEQ ID NO: 8)

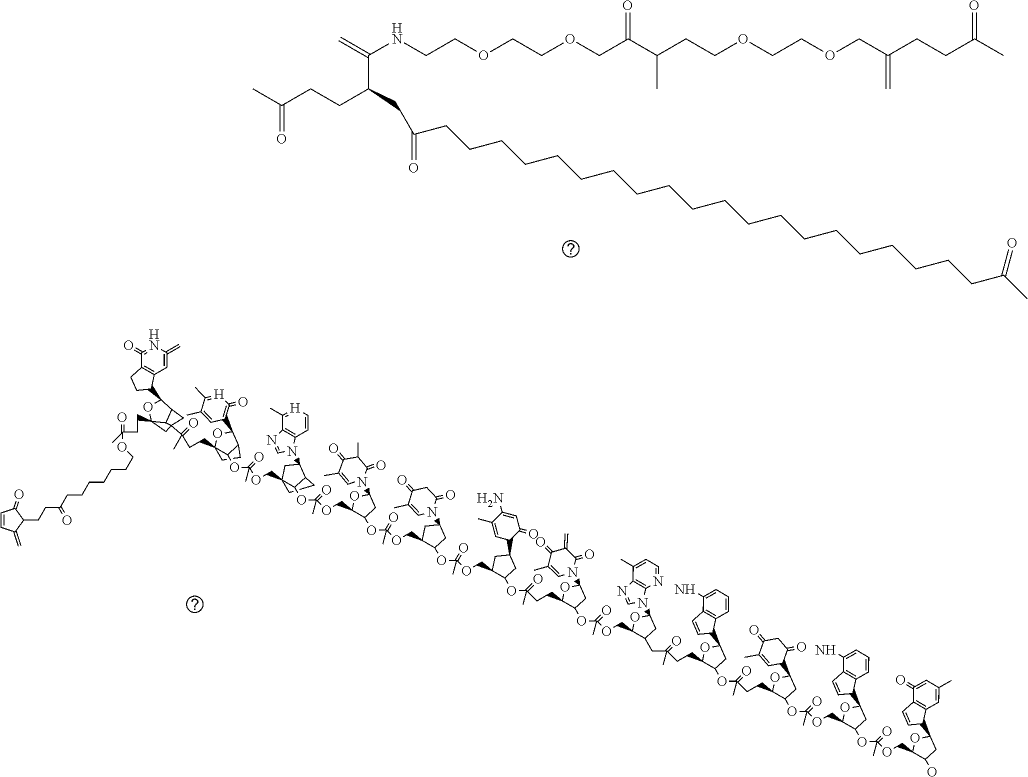

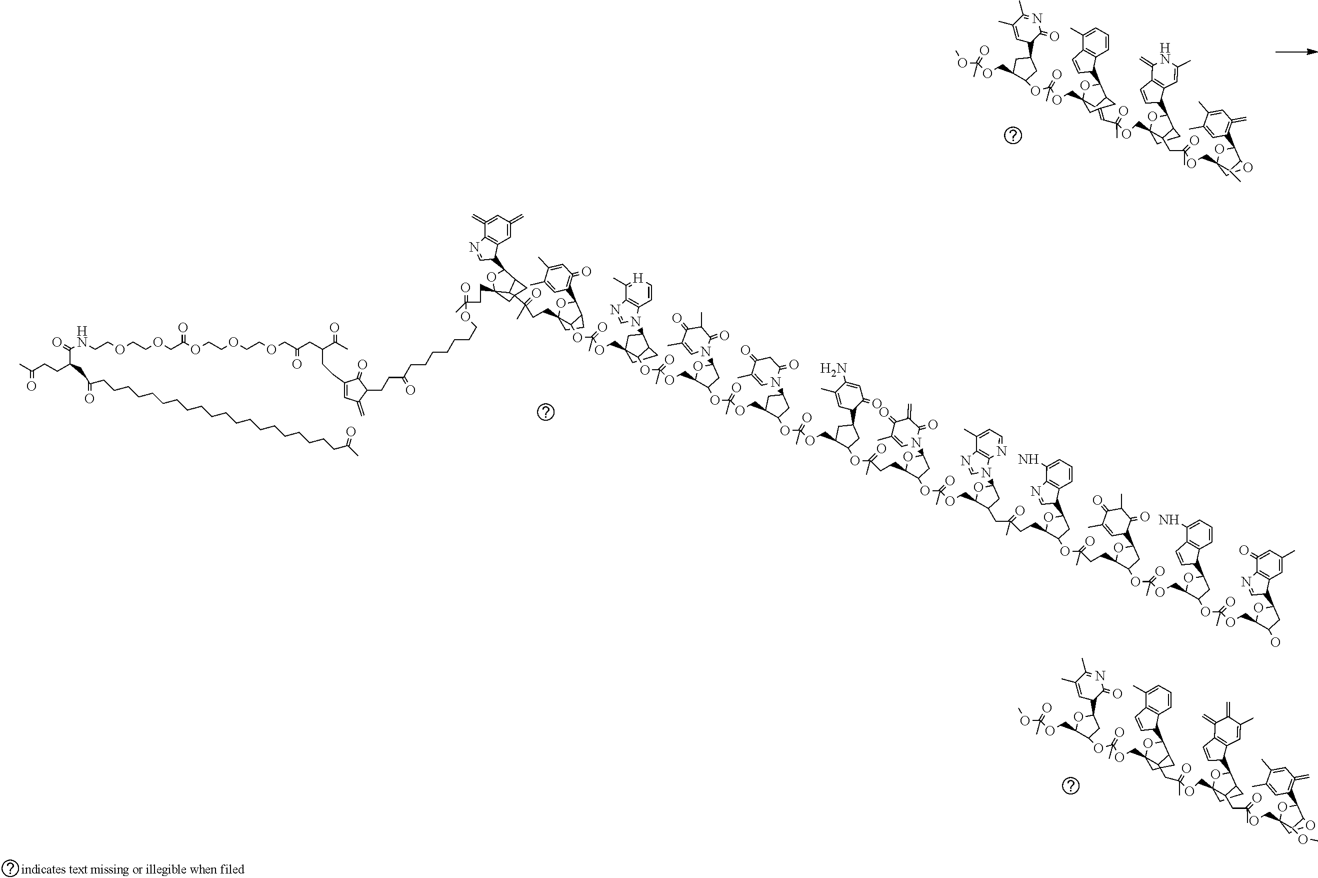

Intermediate 17 (Maleimide-MALAT1-LNA) (32 mg, 5.64 μmol) was dissolved in water (1 mL) and a warm solution of (2R,23S)-1-amino-23-(2-carboxyethyl)-2-(mercaptomethyl)-1,4,13,22,25-pentaoxo-6,9,15,18-tetraoxa-3,12,21,24-tetraazahexatetracontan-46-oic acid (7.54 mg, 8.46 μmol) in DMF/ACN/Water (0.2 ml/0.2 ml/0.05 m) was added. DMF (0.5 mL) was added to get a clear solution. The mixture was stirred for 30 min at 45° C. The product was purified on a XBridge C18, 5 μm 19×150 mm column by using a gradient from 10-90% acetonitrile in NH₄HCO₃ (50 mM, pH8) at R.T. The pure fractions were freeze-dried twice. Yield 12.5 mg (32.3%)

MS (ESI⁻) m/z 1641.2 (z=4)

### Example 25—Determination of Albumin Binding Affinity and Estimation of Free Fraction of 5′ Lipid-Conjugated Malat-1 ASOS Using Surface Plasmon Resonance (SPR) Biosensor

Affinity determination of serum albumin from different species (human, bovine, rat) with Malat1 ASO gapmer carrying different ligands was achieved using a Biacore 5200 (Cytiva). All experiments were performed at 20° C. on a HLC30M chip (Xantec) using running buffer: 10 mM HEPES, pH 7.4, 150 mM NaCl at 30 μl/min. Albumin was covalently immobilized on the chip by EDC-NHS-activated coupling by injecting 100 nM albumin in 10 mM acetate buffer, pH 5.0. Reference flow cell lacked immobilized protein. Analyte solutions were injected as three-fold serial dilutions up to 150 μM in running buffer, 11 concentrations per analyte, in triplicates. Data were evaluated with Biacore S200 evaluation software (Cytiva), and the obtained sensorgrams were fitted into a 1:1 steady-state affinity (fixed Rmax). The resulting apparent affinity (K_{d, app}) is used to estimate the free fraction (fu) according to

$$fu = 1 - \frac{{[ L ]}_{bound}}{{[ L ]}_{tot}} = 1 - \frac{[ R ]}{α {K}_{d , app} + [ R ]}$$

where [R] is the plasma concentration of albumin and a is an assay calibration factor.

Estimation assumes that the albumin interaction is the dominant contributor to plasma protein binding and that [R]=[R]ₜₒₜ (i.e. [R]>>[L]).

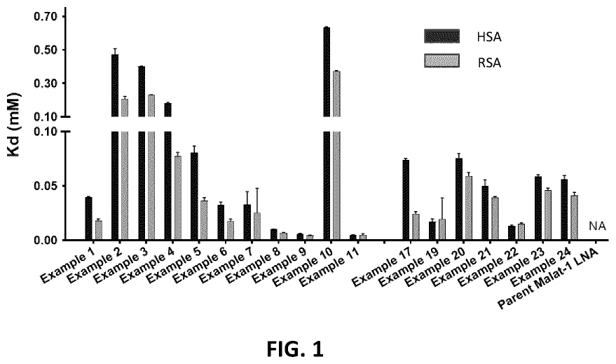

The data in FIG. 1 indicated that the increased affinity of lipid-ASO conjugates for albumin correlates well with the increased lipophilicity/chain length of the fatty acid part. This can be further modulated by the introduction of unsaturation on the fatty acid chain but also by modifying linker and spacer properties (lipophilicity and introduction of additional polar groups) between the oligonucleotide and the fatty acid chain. The observed modulation of albumin affinity based on differences in lipids and linker composition translated well across the species studied.

### Example 26—Determination of Knock Down Efficiency of Selected 5′ Lipid-Conjugated MALAT-1 ASOS in THP-1 Cells

THP-1 cells (ATCC® TIB-202™) were cultured according to standard procedures in RPMI 1640 with GlutaMax, 2 g/L glucose, HEPES, MEM Non-Essential Amino Acids, 1 mM sodium pyruvate, 10% FBS and 50 μM β-mercaptoethanol. Cells were collected by centrifugation, resuspended in serum free medium and plated at 70.000 cells per well in 96 well culture plates. FA-ASO conjugates were dosed into the medium at final concentrations 1, 0.3, 0.1, and 0.03 μM.

Cells were incubated at 37° C., 5% CO₂ for 24 hours.

After incubation, cells were transferred to 96-well V-bottom polypropylene microplates and pelleted at 500 g for 3 minutes. Medium was removed and cells lysed in 20 μL lysis buffer (Qiagen RNeasy RLN lysis buffer with 4% RNAsecure™ RNase Inactivation Reagent from Invitrogen) for 5 minutes at room temperature. 2 μL of lysates were used as templates in 20 μL-reverse transcription (RT) reactions (50% RT buffer and 5% enzyme mix from Invitrogen's Cells-to-CT Bulk RT Reagents), and RT was performed at 37° C. for 60 min, then 95° C. for 5 min.

The cDNA samples were diluted 1:4 and Real-Time PCR reactions were set up using 3 μL cDNA, TaqMan™ Fast Advanced Master Mix, and Malat-1 or GAPDH TaqMan™ Gene Expression Assays (Hs00273907_s1 and Hs99999905_ml, all Applied Biosystems) in a total volume of 10 μL. Amplifications were performed on a QuantStudio™ 7 Flex Real-Time PCR System (Applied Biosystems) and were conducted at 50° C. for 2 min, 95° C. for 10 min, followed by 40 cycles of 95° C. for 15 s and 60° C. for 1 min. Quantification cycle (Cq) values were determined by the software using the Auto Baseline and Auto Threshold options and were then used to calculate relative Malat-1 expression (2\hat{ }-dCq) normalized against the reference gene GAPDH.s

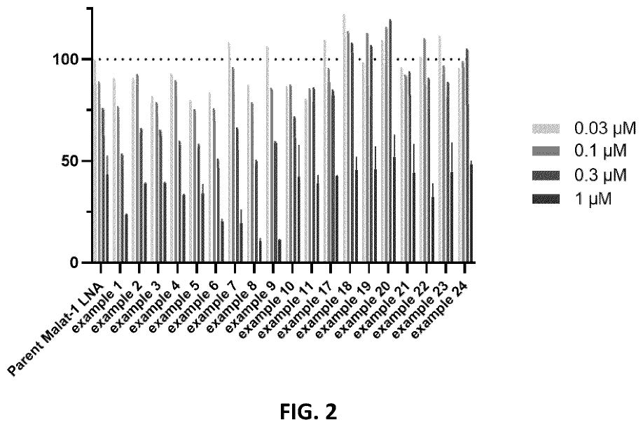

The data in FIG. 2 indicated that the introduction in the 5′ position of an ASO of a saturated fatty acid chain bearing a carboxylic acid group improved in vitro knock down of Malat-1 in THP-1 cells. The increased functional activity and correlated with increasing lipophilicty of the lipid component. Variation of the linker and spacer chemistries can modulate the in vitro activity.

### Example 27—Determination of Knock Down Efficiency of Selected 5′ Lipid-Conjugated CamK2D ASOS in LA-4 Cells

LA-4 cells (ATCC CCL-196 ® ™) were cultured according to standard procedures in Ham's F12 nutrient mix media with GlutaMax, 1.8 g/L glucose, 1% MEM Non-Essential Amino Acids and 15% FBS. Cells were trypsinised, resuspended in standard culture medium and plated at 6000 cells per well in 384 well culture plates. 16 h later the media was removed from the cells and replaced with serum free media. 12-point dilution series (concentration range 0.000085-15 μM) were prepared for both naked and C22 lipid conjugated ASOs. These were dosed into the medium and cells were incubated at 37° C., 5% CO₂ for 24 hours.

After incubation, medium was removed, cells were washed in PBS and lysed in 10 μL Cells to CT lysis buffer (Life Technologies)+1% DNAse per well for 5 minutes at room temperature with shaking. 2 ul of Cells to CT stop solution (Life Technologies) was then added per well. 4 μL of each cell lysate was used a template in a 9 μL reverse transcription (RT) reactions (50% RT buffer and 5% enzyme mix from Invitrogen's Cells-to-CT Bulk RT Reagents), and RT was performed at 37° C. for 30 min, followed by RT inactivation at 95° C. for 5 min.

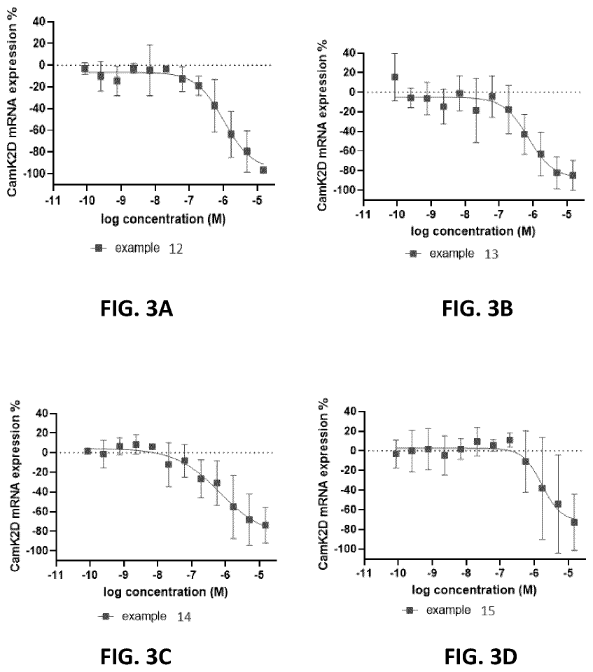

Real-Time PCR reactions were set up using 3 μL cDNA, TaqMan™ Fast Advanced Master Mix, and Malat-1 or Rplp0 TaqMan™ Gene Expression Assays (Mm00499266_ml and Mm00725448_s1, all Applied Biosystems) in a total volume of 10 μL. Amplifications were performed on a QuantStudio™ 7 Flex Real-Time PCR System (Applied Biosystems) and were conducted at 50° C. for 2 min, 95° C. for 10 min, followed by 40 cycles of 95° C. for 15 s and 60° C. for 1 min. Quantification cycle (Cq) values were determined by the software using the Auto Baseline and Auto Threshold options and were then used to calculate relative CamK2D expression (2\hat{ }-dCq) normalized against the reference gene Rplp0. 2\hat{ }-dCq values for CamK2D were then normalized to values obtained from H₂O treated samples. The data in FIGS. 3A-D indicates that the lipidated CamK2D ASOs maintained functional activity in vitro.

### Example 28—Knockdown Effect of Fatty-Acid-Conjugated Malat-1 ASOS on the Target Gene in B6NTAC Mice

Male B6NTac mice arrived at 8-10 weeks of age, 2/cage-housed and placed on Chow Diet. Mice were allowed to acclimate for at least one week before subjected to the study. On day −1, mice were weighed and randomized to appropriate drug treatment groups based on the body weight. Compounds were dosed by subcutaneous injection through tail vein at 5 mg/Kg in PBS at pH7.4 at days 0, 2, 4, 7 and 21. Mice were euthanized via CO₂ inhalation at day 28. The hearts were removed and approximately 20 mg of apex were weighed out for RNA extraction. One piece of the liver left lobe was snap frozen in liquid nitrogen for RNA extraction. The right kidney were removed and snap frozen for RNA extraction. Knock-down effect was evaluated by RT-qPCR as described in example 26.

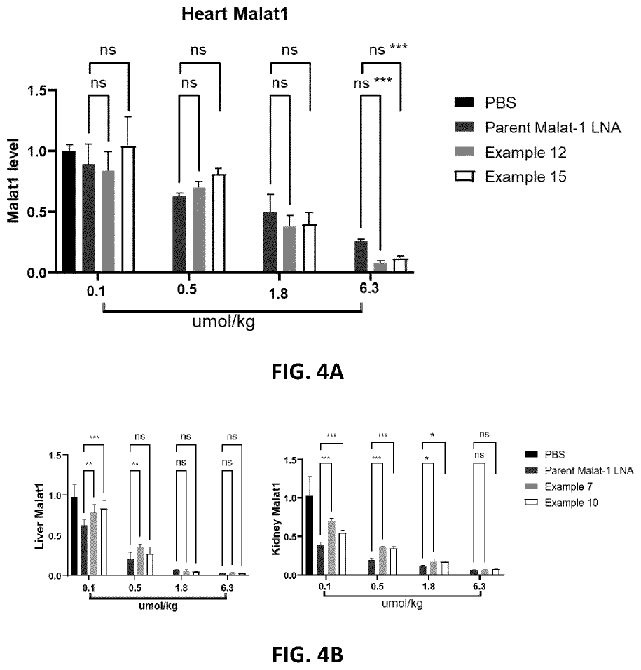

The data indicated in FIGS. 4A-B show that conjugation to a saturated C₂₂ acid or Cis (9Z) monounsaturated fatty diacid led to similar or increased knock down in the heart but also to an attenuation of the knock down measured in the liver and kidney compared to the parent ASO.

### Example 29—Knockdown Effect of Fatty-Acid-Conjugated Camk2D ASOS on the Target Gene in B6NTAC Mice

Male B6NTac mice arrived at 8-10 weeks of age, 2/cage-housed and placed on Chow Diet. Mice were allowed to acclimate for at least one week before subjected to the study. On day 0, mice were weighed and randomized to appropriate drug treatment groups based on the body weight. On day 1 compounds were dosed by subcutaneous injection through tail vein at 5 mg/Kg in PBS at pH7.4. Mice were euthanized via CO₂ inhalation at day 4. The hearts were removed and approximately 20 mg of apex were weighed out for RNA extraction. One piece of the liver left lobe was snap frozen in liquid nitrogen for RNA extraction. The right kidney was removed and snap frozen for RNA extraction. Knock-down effect was evaluated as described in example 26.

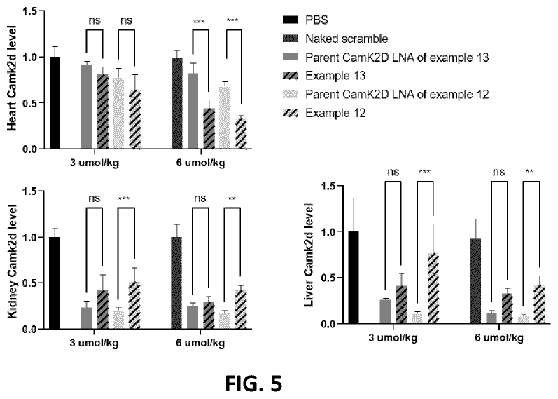

The data indicated in FIG. 5 shows that the saturated C₂₂ acid chain conjugation improved knock down in the heart and tend to attenuate the knock-down in sink organs like kidney and liver in comparison to the naked parent ASOs.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

SEQUENCES

| SEQ | |
|---|---|
| ID | |
| NO | Sequence |
| 1 | cgcagcctgc agcccgagac ttctgtaaag gactggggcc ccgcaactgg cctctcctgc |
| | cctcttaagc gcagcgccat tttagcaacg cagaagcccg gcgccgggaa gcctcagctc |
| | gcctgaaggc aggtcccctc tgacgcctcc gggagcccag gtttcccaga gtccttggga |
| | cgcagcgacg agttgtgctg ctatcttagc tgtccttata ggctggccat tccaggtggt |
| | ggtatttaga taaaaccact caaactctgc agtttggtct tggggtttgg aggaaagctt |
| | ttatttttct tcctgctccg gttcagaagg tctgaagctc atacctaacc aggcataaca |
| | cagaatctgc aaaacaaaaa cccctaaaaa agcagaccca gagcagtgta aacacttctg |
| | ggtgtgtccc tgactggctg cccaaggtct ctgtgtcttc ggagacaaag ccattcgctt |
| | agttggtcta ctttaaaagg ccacttgaac tcgctttcca tggcgatttg ccttgtgagc |
| | actttcagga gagcctggaa gctgaaaaac ggtagaaaaa tttccgtgcg ggccgtgggg |
| | ggctggcggc aactgggggg ccgcagatca gagtgggcca ctggcagcca acggcccccg |
| | gggctcaggc ggggagcagc tctgtggtgt gggattgagg cgttttccaa gagtgggttt |
| | tcacgtttct aagatttccc aagcagacag cccgtgctgc tccgatttct cgaacaaaaa |
| | agcaaaacgt gtggctgtct tgggagcaag tcgcaggact gcaagcagtt gggggagaaa |
| | gtccgccatt ttgccacttc tcaaccgtcc ctgcaaggct ggggctcagt tgcgtaatgg |
| | aaagtaaagc cctgaactat cacactttaa tcttccttca aaaggtggta aactatacct |
| | actgtccctc aagagaacac aagaagtgct ttaagaggta ttttaaaagt tccgggggtt |
| | ttgtgaggtg tttgatgacc cgtttaaaat atgatttcca tgtttctttt gtctaaagtt |
| | tgcagctcaa atctttccac acgctagtaa tttaagtatt tctgcatgtg tagtttgcat |
| | tcaagttcca taagctgtta agaaaaatct agaaaagtaa aactagaacc tatttttaac |
| | cgaagaacta ctttttgcct ccctcacaaa ggcggcggaa ggtgatcgaa ttccggtgat |
| | gcgagttgtt ctccgtctat aaatacgcct cgcccgagct gtgcggtagg cattgaggca |
| | gccagcgcag gggcttctgc tgagggggca ggcggagctt gaggaaaccg cagataagtt |
| | tttttctctt tgaaagatag agattaatac aactacttaa aaaatatagt caataggtta |
| | ctaagatatt gcttagcgtt aagtttttaa cgtaatttta atagcttaag attttaagag |
| | aaaatatgaa gacttagaag agtagcatga ggaaggaaaa gataaaaggt ttctaaaaca |
| | tgacggaggt tgagatgaag cttcttcatg gagtaaaaaa tgtatttaaa agaaaattga |
| | gagaaaggac tacagagccc cgaattaata ccaatagaag ggcaatgctt ttagattaaa |
| | atgaaggtga cttaaacagc ttaaagttta gtttaaaagt tgtaggtgat taaaataatt |
| | tgaaggcgat cttttaaaaa gagattaaac cgaaggtgat taaaagacct tgaaatccat |
| | gacgcaggga gaattgcgtc atttaaagcc tagttaacgc atttactaaa cgcagacgaa |
| | aatggaaaga ttaattggga gtggtaggat gaaacaattt ggagaagata gaagtttgaa |
| | gtggaaaact ggaagacaga agtacgggaa ggcgaagaaa agaatagaga agatagggaa |
| | attagaagat aaaaacatac ttttagaaga aaaaagataa atttaaacct gaaaagtagg |
| | aagcagaaga aaaaagacaa gctaggaaac aaaaagctaa gggcaaaatg tacaaactta |
| | gaagaaaatt ggaagataga aacaagatag aaaatgaaaa tattgtcaag agtttcagat |
| | agaaaatgaa aaacaagcta agacaagtat tggagaagta tagaagatag aaaaatataa |
| | agccaaaaat tggataaaat agcactgaaa aaatgaggaa attattggta accaatttat |
| | tttaaaagcc catcaattta atttctggtg gtgcagaagt tagaaggtaa agcttgagaa |
| | gatgagggtg tttacgtaga ccagaaccaa tttagaagaa tacttgaagc tagaagggga |
| | agttggttaa aaatcacatc aaaaagctac taaaaggact ggtgtaattt aaaaaaaact |
| | aaggcagaag gcttttggaa gagttagaag aatttggaag gccttaaata tagtagctta |
| | gtttgaaaaa tgtgaaggac tttcgtaacg gaagtaattc aagatcaaga gtaattacca |
| | acttaatgtt tttgcattgg actttgagtt aagattattt tttaaatcct gaggactagc |
| | attaattgac agctgaccca ggtgctacac agaagtggat tcagtgaatc taggaagaca |
| | gcagcagaca ggattccagg aaccagtgtt tgatgaagct aggactgagg agcaagcgag |
| | caagcagcag ttcgtggtga agataggaaa agagtccagg agccagtgcg atttggtgaa |
| | ggaagctagg aagaaggaag gagcgctaac gatttggtgg tgaagctagg aaaaaggatt |
| | ccaggaagga gcgagtgcaa tttggtgatg aaggtagcag gcggcttggc ttggcaacca |
| | cacggaggag gcgagcaggc gttgtgcgta gaggatccta gaccagcatg ccagtgtgcc |
| | aaggccacag ggaaagcgag tggttggtaa aaatccgtga ggtcggcaat atgttgtttt |
| | tctggaactt acttatggta accttttatt tattttctaa tataatgggg gagtttcgta |
| | ctgaggtgta aagggattta tatggggacg taggccgatt tccgggtgtt gtaggtttct |
| | ctttttcagg cttatactca tgaatcttgt ctgaagcttt tgagggcaga ctgccaagtc |
| | ctggagaaat agtagatggc aagtttgtgg gttttttttt tttacacgaa tttgaggaaa |
| | accaaatgaa tttgatagcc aaattgagac aatttcagca aatctgtaag cagtttgtat |
| | gtttagttgg ggtaatgaag tatttcagtt ttgtgaatag atgacctgtt tttacttcct |
| | caccctgaat tcgttttgta aatgtagagt ttggatgtgt aactgaggcg ggggggagtt |
| | ttcagtattt ttttttgtgg gggtgggggc aaaatatgtt ttcagttctt tttcccttag |
| | gtctgtctag aatcctaaag gcaaatgact caaggtgtaa cagaaaacaa gaaaatccaa |
| | tatcaggata atcagaccac cacaggttta cagtttatag aaactagagc agttctcacg |
| | ttgaggtctg tggaagagat gtccattgga gaaatggctg gtagttactc ttttttcccc |
| | ccaccccctt aatcagactt taaaagtgct taacccctta aacttgttat tttttacttg |
| | aagcattttg ggatggtctt aacagggaag agagagggtg ggggagaaaa tgtttttttc |
| | taagattttc cacagatgct atagtactat tgacaaactg ggttagagaa ggagtgtacc |
| | gctgtgctgt tggcacgaac accttcaggg actggagctg cttttatcct tggaagagta |
| | ttcccagttg aagctgaaaa gtacagcaca gtgcagcttt ggttcatatt cagtcatctc |
| | aggagaactt cagaagagct tgagtaggcc aaatgttgaa gttaagtttt ccaataatgt |
| | gacttcttaa aagttttatt aaaggggagg ggcaaatatt ggcaattagt tggcagtggc |
| | ctgttacggt tgggattggt ggggtgggtt taggtaattg tttagtttat gattgcagat |
| | aaactcatgc cagagaactt aaagtcttag aatggaaaaa gtaaagaaat atcaacttcc |
| | aagttggcaa gtaactccca atgatttagt ttttttcccc ccagtttgaa ttgggaagct |
| | gggggaagtt aaatatgagc cactgggtgt accagtgcat taatttgggc aaggaaagtg |
| | tcataatttg atactgtatc tgttttcctt caaagtatag agcttttggg gaaggaaagt |
| | attgaactgg gggttggtct ggcctactgg gctgacatta actacaatta tgggaaatgc |
| | aaaagttgtt tggatatggt agtgtgtggt tctcttttgg aatttttttc aggtgattta |
| | ataataattt aaaactacta tagaaactgc agagcaaagg aagtggctta atgatcctga |
| | agggatttct tctgatggta gcttttgtat tatcaagtaa gattctattt tcagttgtgt |
| | gtaagcaagt ttttttttag tgtaggagaa atacttttcc attgtttaac tgcaaaacaa |
| | gatgttaagg tatgcttcaa aaattttgta aattgtttat tttaaactta tctgtttgta |
| | aattgtaact gattaagaat tgtgatagtt cagcttgaat gtctcttaga gggtgggctt |
| | ttgttgatga gggaggggaa actttttttt tttctataga cttttttcag ataacatctt |
| | ctgagtcata accagcctgg cagtatgatg gcctagatgc agagaaaaca gctccttggt |
| | gaattgataa gtaaaggcag aaaagattat atgtcatacc tccattgggg aataagcata |
| | accctgagat tcttactact gatgagaaca ttatctgcat atgccaaaaa attttaagca |
| | aatgaaagct accaatttaa agttacggaa tctaccattt taaagttaat tgcttgtcaa |
| | gctataacca caaaaataat gaattgatga gaaatacaat gaagaggcaa tgtccatctc |
| | aaaatactgc ttttacaaaa gcagaataaa agcgaaaaga aatgaaaatg ttacactaca |
| | ttaatcctgg aataaaagaa gccgaaataa atgagagatg agttgggatc aagtggattg |
| | aggaggctgt gctgtgtgcc aatgtttcgt ttgcctcaga caggtatctc ttcgttatca |
| | gaagagttgc ttcatttcat ctgggagcag aaaacagcag gcagctgtta acagataagt |
| | ttaacttgca tctgcagtat tgcatgttag ggataagtgc ttatttttaa gagctgtgga |
| | gttcttaaat atcaaccatg gcactttctc ctgacccctt ccctagggga tttcaggatt |
| | gagaaatttt tccatcgagc ctttttaaaa ttgtaggact tgttcctgtg ggcttcagtg |
| | atgggatagt acacttcact cagaggcatt tgcatcttta aataatttct taaaagcctc |
| | taaagtgatc agtgccttga tgccaactaa ggaaatttgt ttagcattga atctctgaag |
| | gctctatgaa aggaatagca tgatgtgctg ttagaatcag atgttactgc taaaatttac |
| | atgttgtgat gtaaattgtg tagaaaacca ttaaatcatt caaaataata aactattttt |
| | attagagaat gtatactttt agaaagctgt ctccttattt aaataaaata gtgtttgtct |
| | gtagttcagt gttggggcaa tcttgggggg gattcttctc taatctttca gaaactttgt |
| | ctgcgaacac tctttaatgg accagatcag gatttgagcg gaagaacgaa tgtaacttta |
| | aggcaggaaa gacaaatttt attcttcata aagtgatgag catataataa ttccaggcac |
| | atggcaatag aggccctcta aataaggaat aaataacctc ttagacaggt gggagattat |
| | gatcagagta aaaggtaatt acacatttta tttccagaaa gtcaggggtc tataaattga |
| | cagtgattag agtaatactt tttcacattt ccaaagtttg catgttaact ttaaatgctt |
| | acaatcttag agtggtaggc aatgttttac actattgacc ttatataggg aagggagggg |
| | gtgcctgtgg ggttttaaag aattttcctt tgcagaggca tttcatcctt catgaagcca |
| | ttcaggattt tgaattgcat atgagtgctt ggctcttcct tctgttctag tgagtgtatg |
| | agaccttgca gtgagtttat cagcatactc aaaatttttt tcctggaatt tggagggatg |
| | ggaggagggg gtggggctta cttgttgtag cttttttttt ttttacagac ttcacagaga |
| | atgcagttgt cttgacttca ggtctgtctg ttctgttggc aagtaaatgc agtactgttc |
| | tgatcccgct gctattagaa tgcattgtga aacgactgga gtatgattaa aagttgtgtt |
| | ccccaatgct tggagtagtg attgttgaag gaaaaaatcc agctgagtga taaaggctga |
| | gtgttgagga aatttctgca gttttaagca gtcgtatttg tgattgaagc tgagtacatt |
| | ttgctggtgt atttttaggt aaaatgcttt ttgttcattt ctggtggtgg gaggggactg |
| | aagcctttag tcttttccag atgcaacctt aaaatcagtg acaagaaaca ttccaaacaa |
| | gcaacagtct tcaagaaatt aaactggcaa gtggaaatgt ttaaacagtt cagtgatctt |
| | tagtgcattg tttatgtgtg ggtttctctc tcccctccct tggtcttaat tcttacatgc |
| | aggaacactc agcagacaca cgtatgcgaa gggccagaga agccagaccc agtaagaaaa |
| | aatagcctat ttactttaaa taaaccaaac attccatttt aaatgtgggg attgggaacc |
| | actagttctt tcagatggta ttcttcagac tatagaagga gcttccagtt gaattcacca |
| | gtggacaaaa tgaggaaaac aggtgaacaa gctttttctg tatttacata caaagtcaga |
| | tcagttatgg gacaatagta ttgaatagat ttcagcttta tgctggagta actggcatgt |
| | gagcaaactg tgttggcgtg ggggtggagg ggtgaggtgg gcgctaagcc tttttttaag |
| | atttttcagg tacccctcac taaaggcacc gaaggcttaa agtaggacaa ccatggagcc |
| | ttcctgtggc aggagagaca acaaagcgct attatcctaa ggtcaagaga agtgtcagcc |
| | tcacctgatt tttattagta atgaggactt gcctcaactc cctctttctg gagtgaagca |
| | tccgaaggaa tgcttgaagt acccctgggc ttctcttaac atttaagcaa gctgttttta |
| | tagcagctct taataataaa gcccaaatct caagcggtgc ttgaagggga gggaaagggg |
| | gaaagcgggc aaccactttt ccctagcttt tccagaagcc tgttaaaagc aaggtctccc |
| | cacaagcaac ttctctgcca catcgccacc ccgtgccttt tgatctagca cagacccttc |
| | acccctcacc tcgatgcagc cagtagcttg gatccttgtg ggcatgatcc ataatcggtt |
| | tcaaggtaac gatggtgtcg aggtctttgg tgggttgaac tatgttagaa aaggccatta |
| | atttgcctgc aaattgttaa cagaagggta ttaaaaccac agctaagtag ctctattata |
| | atacttatcc agtgactaaa accaacttaa accagtaagt ggagaaataa catgttcaag |
| | aactgtaatg ctgggtggga acatgtaact tgtagactgg agaagatagg catttgagtg |
| | gctgagaggg cttttgggtg ggaatgcaaa aattctctgc taagactttt tcaggtgaac |
| | ataacagact tggccaagct agcatcttag cggaagctga tctccaatgc tcttcagtag |
| | ggtcatgaag gtttttcttt tcctgagaaa acaacacgta ttgttttctc aggttttgct |
| | ttttggcctt tttctagctt aaaaaaaaaa aaagcaaaag atgctggtgg ttggcactcc |
| | tggtttccag gacggggttc aaatccctgc ggcgtctttg ctttgactac taatctgtct |
| | tcaggactct ttctgtattt ctccttttct ctgcaggtgc tagttcttgg agttttgggg |
| | aggtgggagg taacagcaca atatctttga actatataca tccttgatgt ataatttgtc |
| | aggagcttga cttgattgta tattcatatt tacacgagaa cctaatataa ctgccttgtc |
| | tttttcaggt aatagcctgc agctggtgtt ttgagaagcc ctactgctga aaacttaaca |
| | attttgtgta ataaaaatgg agaagctcta aattgttgtg gttcttttgt gaataaaaaa |
| | atcttgattg gggaaaaaa |
| 2 | cgcagcctgc agcccgagac ttctgtaaag gactggggcc ccgcaactgg cctctcctgc |
| | cctcttaagc gcagcgccat tttagcaacg cagaagcccg gcgccgggaa gcctcagctc |
| | gcctgaaggc aggtcccctc tgacgcctcc gggagcccag gtttcccaga gtccttggga |
| | cgcagcgacg agttgtgctg ctatcttagc tgtccttata ggctggccat tccaggtggt |
| | ggtatttaga taaaaccact caaactctgc agtttggtct tggggtttgg aggaaagctt |
| | ttatttttct tcctgctccg gttcagaagg tctgaagctc atacctaacc aggcataaca |
| | cagaatctgc aaaacaaaaa cccctaaaaa agcagaccca gagcagtgta aacacttctg |
| | ggtgtgtccc tgactggctg cccaaggtct ctgtgtcttc ggagacaaag ccattcgctt |
| | agttggtcta ctttaaaagg ccacttgaac tcgctttcca tggcgatttg ccttgtgagc |
| | actttcagga gagcctggaa gctgaaaaac ggtagaaaaa tttccgtgcg ggccgtgggg |
| | ggctggcggc aactgggggg ccgcagatca gagtgggcca ctggcagcca acggcccccg |
| | gggctcaggc ggggagcagc tctgtggtgt gggattgagg cgttttccaa gagtgggttt |
| | tcacgtttct aagatttccc aagcagacag cccgtgctgc tccgatttct cgaacaaaaa |
| | agcaaaacgt gtggctgtct tgggagcaag tcgcaggact gcaagcagtt gggggagaaa |
| | gtccgccatt ttgccacttc tcaaccgtcc ctgcaaggct ggggctcagt tgcgtaatgg |
| | aaagtaaagc cctgaactat cacactttaa tcttccttca aaaggtggta aactatacct |
| | actgtccctc aagagaacac aagaagtgct ttaagaggcg gcggaaggtg atcgaattcc |
| | ggtgatgcga gttgttctcc gtctataaat acgcctcgcc cgagctgtgc ggtaggcatt |
| | gaggcagcca gcgcaggggc ttctgctgag ggggcaggcg gagcttgagg aaaccgcaga |
| | taagtttttt tctctttgaa agatagagat taatacaact acttaaaaaa tatagtcaat |
| | aggttactaa gatattgctt agcgttaagt ttttaacgta attttaatag cttaagattt |
| | taagagaaaa tatgaagact tagaagagta gcatgaggaa ggaaaagata aaaggtttct |
| | aaaacatgac ggaggttgag atgaagcttc ttcatggagt aaaaaatgta tttaaaagaa |
| | aattgagaga aaggactaca gagccccgaa ttaataccaa tagaagggca atgcttttag |
| | attaaaatga aggtgactta aacagcttaa agtttagttt aaaagttgta ggtgattaaa |
| | ataatttgaa ggcgatcttt taaaaagaga ttaaaccgaa ggtgattaaa agaccttgaa |
| | atccatgacg cagggagaat tgcgtcattt aaagcctagt taacgcattt actaaacgca |
| | gacgaaaatg gaaagattaa ttgggagtgg taggatgaaa caatttggag aagatagaag |
| | tttgaagtgg aaaactggaa gacagaagta cgggaaggcg aagaaaagaa tagagaagat |
| | agggaaatta gaagataaaa acatactttt agaagaaaaa agataaattt aaacctgaaa |
| | agtaggaagc agaagaaaaa agacaagcta ggaaacaaaa agctaagggc aaaatgtaca |
| | aacttagaag aaaattggaa gatagaaaca agatagaaaa tgaaaatatt gtcaagagtt |
| | tcagatagaa aatgaaaaac aagctaagac aagtattgga gaagtataga agatagaaaa |
| | atataaagcc aaaaattgga taaaatagca ctgaaaaaat gaggaaatta ttggtaacca |
| | atttatttta aaagcccatc aatttaattt ctggtggtgc agaagttaga aggtaaagct |
| | tgagaagatg agggtgttta cgtagaccag aaccaattta gaagaatact tgaagctaga |
| | aggggaagtt ggttaaaaat cacatcaaaa agctactaaa aggactggtg taatttaaaa |
| | aaaactaagg cagaaggctt ttggaagagt tagaagaatt tggaaggcct taaatatagt |
| | agcttagttt gaaaaatgtg aaggactttc gtaacggaag taattcaaga tcaagagtaa |
| | ttaccaactt aatgtttttg cattggactt tgagttaaga ttatttttta aatcctgagg |
| | actagcatta attgacagct gacccaggtg ctacacagaa gtggattcag tgaatctagg |
| | aagacagcag cagacaggat tccaggaacc agtgtttgat gaagctagga ctgaggagca |
| | agcgagcaag cagcagttcg tggtgaagat aggaaaagag tccaggagcc agtgcgattt |
| | ggtgaaggaa gctaggaaga aggaaggagc gctaacgatt tggtggtgaa gctaggaaaa |
| | aggattccag gaaggagcga gtgcaatttg gtgatgaagg tagcaggcgg cttggcttgg |
| | caaccacacg gaggaggcga gcaggcgttg tgcgtagagg atcctagacc agcatgccag |
| | tgtgccaagg ccacagggaa agcgagtggt tggtaaaaat ccgtgaggtc ggcaatatgt |
| | tgtttttctg gaacttactt atggtaacct tttatttatt ttctaatata atgggggagt |
| | ttcgtactga ggtgtaaagg gatttatatg gggacgtagg ccgatttccg ggtgttgtag |
| | gtttctcttt ttcaggctta tactcatgaa tcttgtctga agcttttgag ggcagactgc |
| | caagtcctgg agaaatagta gatggcaagt ttgtgggttt ttttttttta cacgaatttg |
| | aggaaaacca aatgaatttg atagccaaat tgagacaatt tcagcaaatc tgtaagcagt |
| | ttgtatgttt agttggggta atgaagtatt tcagttttgt gaatagatga cctgttttta |
| | cttcctcacc ctgaattcgt tttgtaaatg tagagtttgg atgtgtaact gaggcggggg |
| | ggagttttca gtattttttt ttgtgggggt gggggcaaaa tatgttttca gttctttttc |
| | ccttaggtct gtctagaatc ctaaaggcaa atgactcaag gtgtaacaga aaacaagaaa |
| | atccaatatc aggataatca gaccaccaca ggtttacagt ttatagaaac tagagcagtt |
| | ctcacgttga ggtctgtgga agagatgtcc attggagaaa tggctggtag ttactctttt |
| | ttccccccac ccccttaatc agactttaaa agtgcttaac cccttaaact tgttattttt |
| | tacttgaagc attttgggat ggtcttaaca gggaagagag agggtggggg agaaaatgtt |
| | tttttctaag attttccaca gatgctatag tactattgac aaactgggtt agagaaggag |
| | tgtaccgctg tgctgttggc acgaacacct tcagggactg gagctgcttt tatccttgga |
| | agagtattcc cagttgaagc tgaaaagtac agcacagtgc agctttggtt catattcagt |
| | catctcagga gaacttcaga agagcttgag taggccaaat gttgaagtta agttttccaa |
| | taatgtgact tcttaaaagt tttattaaag gggaggggca aatattggca attagttggc |
| | agtggcctgt tacggttggg attggtgggg tgggtttagg taattgttta gtttatgatt |
| | gcagataaac tcatgccaga gaacttaaag tcttagaatg gaaaaagtaa agaaatatca |
| | acttccaagt tggcaagtaa ctcccaatga tttagttttt ttccccccag tttgaattgg |
| | gaagctgggg gaagttaaat atgagccact gggtgtacca gtgcattaat ttgggcaagg |
| | aaagtgtcat aatttgatac tgtatctgtt ttccttcaaa gtatagagct tttggggaag |
| | gaaagtattg aactgggggt tggtctggcc tactgggctg acattaacta caattatggg |
| | aaatgcaaaa gttgtttgga tatggtagtg tgtggttctc ttttggaatt tttttcaggt |
| | gatttaataa taatttaaaa ctactataga aactgcagag caaaggaagt ggcttaatga |
| | tcctgaaggg atttcttctg atggtagctt ttgtattatc aagtaagatt ctattttcag |
| | ttgtgtgtaa gcaagttttt ttttagtgta ggagaaatac ttttccattg tttaactgca |
| | aaacaagatg ttaaggtatg cttcaaaaat tttgtaaatt gtttatttta aacttatctg |
| | tttgtaaatt gtaactgatt aagaattgtg atagttcagc ttgaatgtct cttagagggt |
| | gggcttttgt tgatgaggga ggggaaactt tttttttttc tatagacttt tttcagataa |
| | catcttctga gtcataacca gcctggcagt atgatggcct agatgcagag aaaacagctc |
| | cttggtgaat tgataagtaa aggcagaaaa gattatatgt catacctcca ttggggaata |
| | agcataaccc tgagattctt actactgatg agaacattat ctgcatatgc caaaaaattt |
| | taagcaaatg aaagctacca atttaaagtt acggaatcta ccattttaaa gttaattgct |
| | tgtcaagcta taaccacaaa aataatgaat tgatgagaaa tacaatgaag aggcaatgtc |
| | catctcaaaa tactgctttt acaaaagcag aataaaagcg aaaagaaatg aaaatgttac |
| | actacattaa tcctggaata aaagaagccg aaataaatga gagatgagtt gggatcaagt |
| | ggattgagga ggctgtgctg tgtgccaatg tttcgtttgc ctcagacagg tatctcttcg |
| | ttatcagaag agttgcttca tttcatctgg gagcagaaaa cagcaggcag ctgttaacag |
| | ataagtttaa cttgcatctg cagtattgca tgttagggat aagtgcttat ttttaagagc |
| | tgtggagttc ttaaatatca accatggcac tttctcctga ccccttccct aggggatttc |
| | aggattgaga aatttttcca tcgagccttt ttaaaattgt aggacttgtt cctgtgggct |
| | tcagtgatgg gatagtacac ttcactcaga ggcatttgca tctttaaata atttcttaaa |
| | agcctctaaa gtgatcagtg ccttgatgcc aactaaggaa atttgtttag cattgaatct |
| | ctgaaggctc tatgaaagga atagcatgat gtgctgttag aatcagatgt tactgctaaa |
| | atttacatgt tgtgatgtaa attgtgtaga aaaccattaa atcattcaaa ataataaact |
| | atttttatta gagaatgtat acttttagaa agctgtctcc ttatttaaat aaaatagtgt |
| | ttgtctgtag ttcagtgttg gggcaatctt gggggggatt cttctctaat ctttcagaaa |
| | ctttgtctgc gaacactctt taatggacca gatcaggatt tgagcggaag aacgaatgta |
| | actttaaggc aggaaagaca aattttattc ttcataaagt gatgagcata taataattcc |
| | aggcacatgg caatagaggc cctctaaata aggaataaat aacctcttag acaggtggga |
| | gattatgatc agagtaaaag gtaattacac attttatttc cagaaagtca ggggtctata |
| | aattgacagt gattagagta atactttttc acatttccaa agtttgcatg ttaactttaa |
| | atgcttacaa tcttagagtg gtaggcaatg ttttacacta ttgaccttat atagggaagg |
| | gagggggtgc ctgtggggtt ttaaagaatt ttcctttgca gaggcatttc atccttcatg |
| | aagccattca ggattttgaa ttgcatatga gtgcttggct cttccttctg ttctagtgag |
| | tgtatgagac cttgcagtga gtttatcagc atactcaaaa tttttttcct ggaatttgga |
| | gggatgggag gagggggtgg ggcttacttg ttgtagcttt tttttttttt acagacttca |
| | cagagaatgc agttgtcttg acttcaggtc tgtctgttct gttggcaagt aaatgcagta |
| | ctgttctgat cccgctgcta ttagaatgca ttgtgaaacg actggagtat gattaaaagt |
| | tgtgttcccc aatgcttgga gtagtgattg ttgaaggaaa aaatccagct gagtgataaa |
| | ggctgagtgt tgaggaaatt tctgcagttt taagcagtcg tatttgtgat tgaagctgag |
| | tacattttgc tggtgtattt ttaggtaaaa tgctttttgt tcatttctgg tggtgggagg |
| | ggactgaagc ctttagtctt ttccagatgc aaccttaaaa tcagtgacaa gaaacattcc |
| | aaacaagcaa cagtcttcaa gaaattaaac tggcaagtgg aaatgtttaa acagttcagt |
| | gatctttagt gcattgttta tgtgtgggtt tctctctccc ctcccttggt cttaattctt |
| | acatgcagga acactcagca gacacacgta tgcgaagggc cagagaagcc agacccagta |
| | agaaaaaata gcctatttac tttaaataaa ccaaacattc cattttaaat gtggggattg |
| | ggaaccacta gttctttcag atggtattct tcagactata gaaggagctt ccagttgaat |
| | tcaccagtgg acaaaatgag gaaaacaggt gaacaagctt tttctgtatt tacatacaaa |
| | gtcagatcag ttatgggaca atagtattga atagatttca gctttatgct ggagtaactg |
| | gcatgtgagc aaactgtgtt ggcgtggggg tggaggggtg aggtgggcgc taagcctttt |
| | tttaagattt ttcaggtacc cctcactaaa ggcaccgaag gcttaaagta ggacaaccat |
| | ggagccttcc tgtggcagga gagacaacaa agcgctatta tcctaaggtc aagagaagtg |
| | tcagcctcac ctgattttta ttagtaatga ggacttgcct caactccctc tttctggagt |
| | gaagcatccg aaggaatgct tgaagtaccc ctgggcttct cttaacattt aagcaagctg |
| | tttttatagc agctcttaat aataaagccc aaatctcaag cggtgcttga aggggaggga |
| | aagggggaaa gcgggcaacc acttttccct agcttttcca gaagcctgtt aaaagcaagg |
| | tctccccaca agcaacttct ctgccacatc gccaccccgt gccttttgat ctagcacaga |
| | cccttcaccc ctcacctcga tgcagccagt agcttggatc cttgtgggca tgatccataa |
| | tcggtttcaa ggtaacgatg gtgtcgaggt ctttggtggg ttgaactatg ttagaaaagg |
| | ccattaattt gcctgcaaat tgttaacaga agggtattaa aaccacagct aagtagctct |
| | attataatac ttatccagtg actaaaacca acttaaacca gtaagtggag aaataacatg |
| | ttcaagaact gtaatgctgg gtgggaacat gtaacttgta gactggagaa gataggcatt |
| | tgagtggctg agagggcttt tgggtgggaa tgcaaaaatt ctctgctaag actttttcag |
| | gtgaacataa cagacttggc caagctagca tcttagcgga agctgatctc caatgctctt |
| | cagtagggtc atgaaggttt ttcttttcct gagaaaacaa cacgtattgt tttctcaggt |
| | tttgcttttt ggcctttttc tagcttaaaa aaaaaaaaag caaaagatgc tggtggttgg |
| | cactcctggt ttccaggacg gggttcaaat ccctgcggcg tctttgcttt gactactaat |
| | ctgtcttcag gactctttct gtatttctcc ttttctctgc aggtgctagt tcttggagtt |
| | ttggggaggt gggaggtaac agcacaatat ctttgaacta tatacatcct tgatgtataa |
| | tttgtcagga gcttgacttg attgtatatt catatttaca cgagaaccta atataactgc |
| | cttgtctttt tcaggtaata gcctgcagct ggtgttttga gaagccctac tgctgaaaac |
| | ttaacaattt tgtgtaataa aaatggagaa gctctaaatt gttgtggttc ttttgtgaat |
| | aaaaaaatct tgattgggga aaaaa |
| 3 | cgcagcctgc agcccgagac ttctgtaaag gactggggcc ccgcaactgg cctctcctgc |
| | cctcttaagc gcagcgccat tttagcaacg cagaagcccg gcgccgggaa gcctcagctc |
| | gcctgaaggc aggtcccctc tgacgcctcc gggagcccag gtttcccaga gtccttggga |
| | cgcagcgacg agttgtgctg ctatcttagc tgtccttata ggctggccat tccaggtggt |
| | ggtatttaga taaaaccact caaactctgc agtttggtct tggggtttgg aggaaagctt |
| | ttatttttct tcctgctccg gttcagaagg tctgaagctc atacctaacc aggcataaca |
| | cagaatctgc aaaacaaaaa cccctaaaaa agcagaccca gagcagtgta aacacttctg |
| | ggtgtgtccc tgactggctg cccaaggtct ctgtgtcttc ggagacaaag ccattcgctt |
| | agttggtcta ctttaaaagg ccacttgaac tcgctttcca tggcgatttg ccttgtgagc |
| | actttcagga gagcctggaa gctgaaaaac ggtagaaaaa tttccgtgcg ggccgtgggg |
| | ggctggcggc aactgggggg ccgcagatca gagtgggcca ctggcagcca acggcccccg |
| | gggctcaggc ggggagcagc tctgtggtgt gggattgagg cgttttccaa gagtgggttt |
| | tcacgtttct aagatttccc aagcagacag cccgtgctgc tccgatttct cgaacaaaaa |
| | agcaaaacgt gtggctgtct tgggagcaag tcgcaggact gcaagcagtt gggggagaaa |
| | gtccgccatt ttgccacttc tcaaccgtcc ctgcaaggct ggggctcagt tgcgtaatgg |
| | aaagtaaagc cctgaactat cacactttaa tcttccttca aaaggtggta aactatacct |
| | actgtccctc aagagaacac aagaagtgct ttaagaggcg gcggaaggtg atcgaattcc |
| | ggtgatgcga gttgttctcc gtctataaat acgcctcgcc cgagctgtgc ggtaggcatt |
| | gaggcagcca gcgcaggggc ttctgctgag ggggcaggcg gagcttgagg aaaccgcaga |
| | taagtttttt tctctttgaa agatagagat taatacaact acttaaaaaa tatagtcaat |
| | aggttactaa gatattgctt agcgttaagt ttttaacgta attttaatag cttaagattt |
| | taagagaaaa tatgaagact tagaagagta gcatgaggaa ggaaaagata aaaggtttct |
| | aaaacatgac ggaggttgag atgaagcttc ttcatggagt aaaaaatgta tttaaaagaa |
| | aattgagaga aaggactaca gagccccgaa ttaataccaa tagaagggca atgcttttag |
| | attaaaatga aggtgactta aacagcttaa agtttagttt aaaagttgta ggtgattaaa |
| | ataatttgaa ggcgatcttt taaaaagaga ttaaaccgaa ggtgattaaa agaccttgaa |
| | atccatgacg cagggagaat tgcgtcattt aaagcctagt taacgcattt actaaacgca |
| | gacgaaaatg gaaagattaa ttgggagtgg taggatgaaa caatttggag aagatagaag |
| | tttgaagtgg aaaactggaa gacagaagta cgggaaggcg aagaaaagaa tagagaagat |
| | agggaaatta gaagataaaa acatactttt agaagaaaaa agataaattt aaacctgaaa |
| | agtaggaagc agaagaaaaa agacaagcta ggaaacaaaa agctaagggc aaaatgtaca |
| | aacttagaag aaaattggaa gatagaaaca agatagaaaa tgaaaatatt gtcaagagtt |
| | tcagatagaa aatgaaaaac aagctaagac aagtattgga gaagtataga agatagaaaa |
| | atataaagcc aaaaattgga taaaatagca ctgaaaaaat gaggaaatta ttggtaacca |
| | atttatttta aaagcccatc aatttaattt ctggtggtgc agaagttaga aggtaaagct |
| | tgagaagatg agggtgttta cgtagaccag aaccaattta gaagaatact tgaagctaga |
| | aggggaagtt ggttaaaaat cacatcaaaa agctactaaa aggactggtg taatttaaaa |
| | aaaactaagg cagaaggctt ttggaagagt tagaagaatt tggaaggcct taaatatagt |
| | agcttagttt gaaaaatgtg aaggactttc gtaacggaag taattcaaga tcaagagtaa |
| | ttaccaactt aatgtttttg cattggactt tgagttaaga ttatttttta aatcctgagg |
| | actagcatta attgacagct gacccaggtg ctacacagaa gtggattcag tgaatctagg |
| | aagacagcag cagacaggat tccaggaacc agtgtttgat gaagctagga ctgaggagca |
| | agcgagcaag cagcagttcg tggtgaagat aggaaaagag tccaggagcc agtgcgattt |
| | ggtgaaggaa gctaggaaga aggaaggagc gctaacgatt tggtggtgaa gctaggaaaa |
| | aggattccag gaaggagcga gtgcaatttg gtgatgaagg tagcaggcgg cttggcttgg |
| | caaccacacg gaggaggcga gcaggcgttg tgcgtagagg atcctagacc agcatgccag |
| | tgtgccaagg ccacagggaa agcgagtggt tggtaaaaat ccgtgaggtc ggcaatatgt |
| | tgtttttctg gaacttactt atggtaacct tttatttatt ttctaatata atgggggagt |
| | ttcgtactga ggtgtaaagg gatttatatg gggacgtagg ccgatttccg ggtgttgtag |
| | gtttctcttt ttcaggctta tactcatgaa tcttgtctga agcttttgag ggcagactgc |
| | caagtcctgg agaaatagta gatggcaagt ttgtgggttt ttttttttta cacgaatttg |
| | aggaaaacca aatgaatttg atagccaaat tgagacaatt tcagcaaatc tgtaagcagt |
| | ttgtatgttt agttggggta atgaagtatt tcagttttgt gaatagatga cctgttttta |
| | cttcctcacc ctgaattcgt tttgtaaatg tagagtttgg atgtgtaact gaggcggggg |
| | ggagttttca gtattttttt ttgtgggggt gggggcaaaa tatgttttca gttctttttc |
| | ccttaggtct gtctagaatc ctaaaggcaa atgactcaag gtgtaacaga aaacaagaaa |
| | atccaatatc aggataatca gaccaccaca ggtttacagt ttatagaaac tagagcagtt |
| | ctcacgttga ggtctgtgga agagatgtcc attggagaaa tggctggtag ttactctttt |
| | ttccccccac ccccttaatc agactttaaa agtgcttaac cccttaaact tgttattttt |
| | tacttgaagc attttgggat ggtcttaaca gggaagagag agggtggggg agaaaatgtt |
| | tttttctaag attttccaca gatgctatag tactattgac aaactgggtt agagaaggag |
| | tgtaccgctg tgctgttggc acgaacacct tcagggactg gagctgcttt tatccttgga |
| | agagtattcc cagttgaagc tgaaaagtac agcacagtgc agctttggtt catattcagt |
| | catctcagga gaacttcaga agagcttgag taggccaaat gttgaagtta agttttccaa |
| | taatgtgact tcttaaaagt tttattaaag gggaggggca aatattggca attagttggc |
| | agtggcctgt tacggttggg attggtgggg tgggtttagg taattgttta gtttatgatt |
| | gcagataaac tcatgccaga gaacttaaag tcttagaatg gaaaaagtaa agaaatatca |
| | acttccaagt tggcaagtaa ctcccaatga tttagttttt ttccccccag tttgaattgg |
| | gaagctgggg gaagttaaat atgagccact gggtgtacca gtgcattaat ttgggcaagg |
| | aaagtgtcat aatttgatac tgtatctgtt ttccttcaaa gtatagagct tttggggaag |
| | gaaagtattg aactgggggt tggtctggcc tactgggctg acattaacta caattatggg |
| | aaatgcaaaa gttgtttgga tatggtagtg tgtggttctc ttttggaatt tttttcaggt |
| | gatttaataa taatttaaaa ctactataga aactgcagag caaaggaagt ggcttaatga |
| | tcctgaaggg atttcttctg atggtagctt ttgtattatc aaactttttt cagataacat |
| | cttctgagtc ataaccagcc tggcagtatg atggcctaga tgcagagaaa acagctcctt |
| | ggtgaattga taagtaaagg cagaaaagat tatatgtcat acctccattg gggaataagc |
| | ataaccctga gattcttact actgatgaga acattatctg catatgccaa aaaattttaa |
| | gcaaatgaaa gctaccaatt taaagttacg gaatctacca ttttaaagtt aattgcttgt |
| | caagctataa ccacaaaaat aatgaattga tgagaaatac aatgaagagg caatgtccat |
| | ctcaaaatac tgcttttaca aaagcagaat aaaagcgaaa agaaatgaaa atgttacact |
| | acattaatcc tggaataaaa gaagccgaaa taaatgagag atgagttggg atcaagtgga |
| | ttgaggaggc tgtgctgtgt gccaatgttt cgtttgcctc agacaggtat ctcttcgtta |
| | tcagaagagt tgcttcattt catctgggag cagaaaacag caggcagctg ttaacagata |
| | agtttaactt gcatctgcag tattgcatgt tagggataag tgcttatttt taagagctgt |
| | ggagttctta aatatcaacc atggcacttt ctcctgaccc cttccctagg ggatttcagg |
| | attgagaaat ttttccatcg agccttttta aaattgtagg acttgttcct gtgggcttca |
| | gtgatgggat agtacacttc actcagaggc atttgcatct ttaaataatt tcttaaaagc |
| | ctctaaagtg atcagtgcct tgatgccaac taaggaaatt tgtttagcat tgaatctctg |
| | aaggctctat gaaaggaata gcatgatgtg ctgttagaat cagatgttac tgctaaaatt |
| | tacatgttgt gatgtaaatt gtgtagaaaa ccattaaatc attcaaaata ataaactatt |
| | tttattagag aatgtatact tttagaaagc tgtctcctta tttaaataaa atagtgtttg |
| | tctgtagttc agtgttgggg caatcttggg ggggattctt ctctaatctt tcagaaactt |
| | tgtctgcgaa cactctttaa tggaccagat caggatttga gcggaagaac gaatgtaact |
| | ttaaggcagg aaagacaaat tttattcttc ataaagtgat gagcatataa taattccagg |
| | cacatggcaa tagaggccct ctaaataagg aataaataac ctcttagaca ggtgggagat |
| | tatgatcaga gtaaaaggta attacacatt ttatttccag aaagtcaggg gtctataaat |
| | tgacagtgat tagagtaata ctttttcaca tttccaaagt ttgcatgtta actttaaatg |
| | cttacaatct tagagtggta ggcaatgttt tacactattg accttatata gggaagggag |
| | ggggtgcctg tggggtttta aagaattttc ctttgcagag gcatttcatc cttcatgaag |
| | ccattcagga ttttgaattg catatgagtg cttggctctt ccttctgttc tagtgagtgt |
| | atgagacctt gcagtgagtt tatcagcata ctcaaaattt ttttcctgga atttggaggg |
| | atgggaggag ggggtggggc ttacttgttg tagctttttt tttttttaca gacttcacag |
| | agaatgcagt tgtcttgact tcaggtctgt ctgttctgtt ggcaagtaaa tgcagtactg |
| | ttctgatccc gctgctatta gaatgcattg tgaaacgact ggagtatgat taaaagttgt |
| | gttccccaat gcttggagta gtgattgttg aaggaaaaaa tccagctgag tgataaaggc |
| | tgagtgttga ggaaatttct gcagttttaa gcagtcgtat ttgtgattga agctgagtac |
| | attttgctgg tgtattttta ggtaaaatgc tttttgttca tttctggtgg tgggagggga |
| | ctgaagcctt tagtcttttc cagatgcaac cttaaaatca gtgacaagaa acattccaaa |
| | caagcaacag tcttcaagaa attaaactgg caagtggaaa tgtttaaaca gttcagtgat |
| | ctttagtgca ttgtttatgt gtgggtttct ctctcccctc ccttggtctt aattcttaca |
| | tgcaggaaca ctcagcagac acacgtatgc gaagggccag agaagccaga cccagtaaga |
| | aaaaatagcc tatttacttt aaataaacca aacattccat tttaaatgtg gggattggga |
| | accactagtt ctttcagatg gtattcttca gactatagaa ggagcttcca gttgaattca |
| | ccagtggaca aaatgaggaa aacaggtgaa caagcttttt ctgtatttac atacaaagtc |
| | agatcagtta tgggacaata gtattgaata gatttcagct ttatgctgga gtaactggca |
| | tgtgagcaaa ctgtgttggc gtgggggtgg aggggtgagg tgggcgctaa gccttttttt |
| | aagatttttc aggtacccct cactaaaggc accgaaggct taaagtagga caaccatgga |
| | gccttcctgt ggcaggagag acaacaaagc gctattatcc taaggtcaag agaagtgtca |
| | gcctcacctg atttttatta gtaatgagga cttgcctcaa ctccctcttt ctggagtgaa |
| | gcatccgaag gaatgcttga agtacccctg ggcttctctt aacatttaag caagctgttt |
| | ttatagcagc tcttaataat aaagcccaaa tctcaagcgg tgcttgaagg ggagggaaag |
| | ggggaaagcg ggcaaccact tttccctagc ttttccagaa gcctgttaaa agcaaggtct |
| | ccccacaagc aacttctctg ccacatcgcc accccgtgcc ttttgatcta gcacagaccc |
| | ttcacccctc acctcgatgc agccagtagc ttggatcctt gtgggcatga tccataatcg |
| | gtttcaaggt aacgatggtg tcgaggtctt tggtgggttg aactatgtta gaaaaggcca |
| | ttaatttgcc tgcaaattgt taacagaagg gtattaaaac cacagctaag tagctctatt |
| | ataatactta tccagtgact aaaaccaact taaaccagta agtggagaaa taacatgttc |
| | aagaactgta atgctgggtg ggaacatgta acttgtagac tggagaagat aggcatttga |
| | gtggctgaga gggcttttgg gtgggaatgc aaaaattctc tgctaagact ttttcaggtg |
| | aacataacag acttggccaa gctagcatct tagcggaagc tgatctccaa tgctcttcag |
| | tagggtcatg aaggtttttc ttttcctgag aaaacaacac gtattgtttt ctcaggtttt |
| | gctttttggc ctttttctag cttaaaaaaa aaaaaagcaa aagatgctgg tggttggcac |
| | tcctggtttc caggacgggg ttcaaatccc tgcggcgtct ttgctttgac tactaatctg |
| | tcttcaggac tctttctgta tttctccttt tctctgcagg tgctagttct tggagttttg |
| | gggaggtggg aggtaacagc acaatatctt tgaactatat acatccttga tgtataattt |
| | gtcaggagct tgacttgatt gtatattcat atttacacga gaacctaata taactgcctt |
| | gtctttttca ggtaatagcc tgcagctggt gttttgagaa gccctactgc tgaaaactta |
| | acaattttgt gtaataaaaa tggagaagct ctaaattgtt gtggttcttt tgtgaataaa |
| | aaaatcttga ttggggaaaa aa |
| 4 | tcagcattctaatagcagc |
| 5 | tm5cagm5cattm5ctaatagm5cagm5c |
| | m5c - 5-methylcytidine |
| 6 | gcattctaatagcagc |
| 7 | gm5cattm5ctaatagm5cagm5c |
| | m5c - 5-methylcytidine |
| 8 | GM5CAttm5ctaatagm5cAGM5C |
| | m5c- 5-methylcytidine and capital letters are LNA nucleosides |
| 9 | GTTtggtattm5cttTAG |
| | m5c- 5-methylcytidine and capital letters are LNA nucleosides |
| 10 | GTGtm5caam5caam5cm5caTTT |
| | m5c - 5-methylcytidine and capital letters are LNA nucleosides |
| 11 | M5CAM5CAaatttattaaM5CTM5CT |
| | m5c - 5-methylcytidine and capital letters are LNA nucleosides |
| 12 | M5CTGttm5cttm5caAtaATG |
| | m5c - 5-methylcytidine and capital letters are LNA nucleosides |
| 13 | AM5CM5Catgagm5ctataM5CTT |
| | m5c - 5-methylcytidine and capital letters are LNA nucleosides |

1-11. (canceled)

12. An acid acyl conjugated oligonucleotide of formula (I): 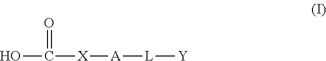
wherein:
X is C₁₄ to C₂₄ alkyl or alkenyl;
A is C═O;
L is a linker 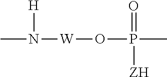
where Z is O or S and W is chosen from C₁ to C₁₀ alkyl or alkenyl or one of the following: 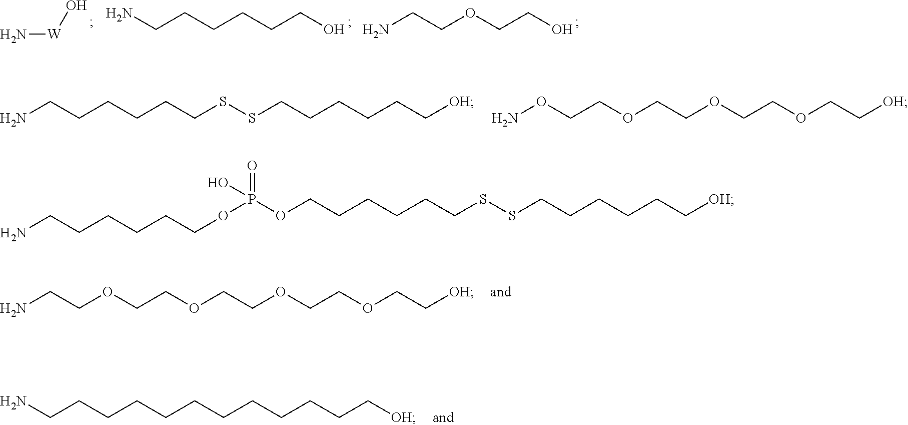
Y is an oligonucleotide.

13-21. (canceled)

22. The acid acyl conjugated oligonucleotide of claim 12, wherein the linker (L) is bound to the 5′ end of the oligonucleotide.

23. The acid acyl conjugated oligonucleotide of claim 12, wherein the linker (L) comprises an amino terminus connected to the carboxy acyl group.

24. The acid acyl conjugated oligonucleotide of claim 12, wherein the linker (L) comprises a phosphate terminus connected to the oligonucleotide.

25. (canceled)

26. The acid acyl conjugated oligonucleotide of claim 12, wherein W is C₄ to C₈ alkyl.

27. The acid acyl conjugated oligonucleotide of claim 12, wherein the linker (L) is —NH—C₆H₁₂—O—PO₂—.

28. The acid acyl conjugated oligonucleotide of claim 12, wherein the linker (L) is attached to the oligonucleotide via a phosphate linkage.

29. The acid acyl conjugated oligonucleotide of claim 12, wherein the oligonucleotide is DNA.

30. The acid acyl conjugated oligonucleotide of claim 12, wherein the oligonucleotide is RNA.

31. The acid acyl conjugated oligonucleotide of claim 12, wherein the oligonucleotide is an antisense oligonucleotide.

32. The acid acyl conjugated oligonucleotide of claim 12, wherein the oligonucleotide is a phosphorothioate oligonucleotide.

33. (canceled)

34. The acid acyl conjugated oligonucleotide of claim 12, wherein the oligonucleotide improves reduction in cardiac cell expression compared to a non-acyl conjugated oligonucleotide.

35-76. (canceled)

77. The acid acyl conjugated oligonucleotide of claim 12, wherein the oligonucleotide is double stranded.


SEQUENCE LISTING (13 sequences)

SEQ ID NO: 1 - DNA, 8779 bp, Homo sapiens
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgatgacccgtttaaaatatgatttccatgtttcttttgtctaaagtttgcagctcaaatctttccacacgctagtaatttaagtatttctgcatgtgtagtttgcattcaagttccataagctgttaagaaaaatctagaaaagtaaaactagaacctatttttaaccgaagaactactttttgcctccctcacaaaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 2 - DNA, 8545 bp, Homo sapiens
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 3 - DNA, 8302 bp, Homo sapiens
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaaacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 4 - DNA, 19 bp, Artificial Sequence
  Synthetic oligonucleotide
tcagcattctaatagcagc

SEQ ID NO: 5 - DNA, 19 bp, Artificial Sequence
  Synthetic oligonucleotide
  modified_base (2)..(2): m5c
  modified_base (5)..(5): m5c
  modified_base (9)..(9): m5c
  modified_base (16)..(16): m5c
  modified_base (19)..(19): m5c
tcagcattctaatagcagc

SEQ ID NO: 6 - DNA, 16 bp, Artificial Sequence
  Synthetic oligonucleotide
gcattctaatagcagc

SEQ ID NO: 7 - DNA, 16 bp, Artificial Sequence
  Synthetic oligonucleotide
  modified_base (2)..(2): m5c
  modified_base (6)..(6): m5c
  modified_base (13)..(13): m5c
  modified_base (16)..(16): m5c
gcattctaatagcagc

SEQ ID NO: 8 - DNA, 16 bp, Artificial Sequence
  Synthetic oligonucleotide
  modified_base (1)..(3): locked nucleic acid
  modified_base (2)..(2): m5c
  modified_base (6)..(6): m5c
  modified_base (13)..(13): m5c
  modified_base (14)..(16): locked nucleic acid
  modified_base (16)..(16): m5c
gcattctaatagcagc

SEQ ID NO: 9 - DNA, 16 bp, Artificial Sequence
  Synthetic oligonucleotide
  modified_base (1)..(3): locked nucleic acid
  modified_base (11)..(11): m5c
  modified_base (14)..(16): locked nucleic acid
gtttggtattctttag

SEQ ID NO: 10 - DNA, 16 bp, Artificial Sequence
  Synthetic oligonucleotide
  modified_base (1)..(3): locked nucleic acid
  modified_base (5)..(5): m5c
  modified_base (8)..(8): m5c
  modified_base (11)..(12): m5c
  modified_base (14)..(16)
gtgtcaacaaccattt

SEQ ID NO: 11 - DNA, 18 bp, Artificial Sequence
  Synthetic oligonucleotide
  modified_base (1)..(4): locked nucleic acid
  modified_base (1)..(1): m5c
  modified_base (3)..(3): m5c
  modified_base (15)..(15): m5c
  modified_base (15)..(18)
  modified_base (17)..(17): m5c
cacaaatttattaactct

SEQ ID NO: 12 - DNA, 16 bp, Artificial Sequence
  Synthetic oligonucleotide
  modified_base (1)..(3): locked nucleic acid
  modified_base (1)..(1): m5c
  modified_base (6)..(6): m5c
  modified_base (9)..(9): m5c
  modified_base (11)..(11): locked nucleic acid
  modified_base (14)..(16): locked nucleic acid
ctgttcttcaataatg

SEQ ID NO: 13 - DNA, 16 bp, Artificial Sequence
  Synthetic oligonucleotide
  modified_base (1)..(3): locked nucleic acid
  modified_base (2)..(3): m5c
  modified_base (9)..(9): m5c
  modified_base (14)..(14): m5c
  modified_base (14)..(16): locked nucleic acid
accatgagctatactt